(19) United States
(12) Patent Application Publication
(10) Pub. No.: US 2020/0375949 A1
(43) Pub. Date: Dec. 3, 2020

(54) BISPHENOL COMPOUNDS

(71) Applicants: THE TRUSTEES OF THE UNIVERSITY OF PENNSYLVANIA, Philadelphia, PA (US); TEMPLE UNIVERSITY - OF THE COMMONWEALTH SYSTEM OF HIGHER EDUCATION, Philadelphia, PA (US); EMORY UNIVERSITY, Atlanta, GA (US)

(72) Inventors: Marisa C. KOZLOWSKI, Wynnewood, PA (US); Young Eun LEE, Elkins Park, PA (US); Thomas PANIAK, Blandon, PA (US); Cristian OCHOA, Philadelphia, PA (US); William M. WUEST, Atlanta, GA (US); Amy E. SOLINSKI, Berlin, NJ (US)

(21) Appl. No.: 16/766,419

(22) PCT Filed: Nov. 21, 2018
(86) PCT No.: PCT/US2018/062227

§ 371 Date: May 22, 2020

Related U.S. Application Data
(60) Provisional application No. 62/589,890, filed on Nov. 22, 2017.

(51) Int. Cl. A61K 31/415; A61K 31/047; A61K 31/09; A61K 31/137; A61K 31/165; A61K 8/49; A61K 8/34; A61K 8/42; A61K 8/41

(52) U.S. Cl. CPC A61K 31/415; A61K 31/047; A61K 31/09; A61K 31/137; A61K 8/41; A61K 8/4946; A61K 8/347; A61K 8/42; A61K 31/165

(57) ABSTRACT

The present disclosure provides compounds of formula (I) or salts thereof, wherein R¹-R¹⁰ and x are defined herein; compositions containing these compounds; methods of inhibiting, reducing, or ameliorating bacterial growth on a substrate using these compound; and products such as dental care products, soaps, antibacterial products, and plastics comprise one or more compounds described herein.

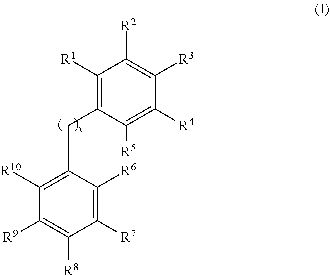

### CROSS-REFERENCE TO RELATED APPLICATIONS

This application claims the benefit of U.S. Provisional Patent Application No. 62/589,890, “Bisphenol Compounds” (filed Nov. 22, 2017), the disclosure of which is incorporated by reference herein in its entirety for any and all purposes.

### GOVERNMENT RIGHTS

This invention was made with government support under Contract Nos. GM112684, DE025837, 1510RR023444, 1510RR022442, 15100D011980, and T32 GM071339 awarded by the National Institutes of Health and Contract Nos. CHE464778, CHE755698, CHE 0840438, and CHE-0848460 awarded by the National Science Foundation. The government has certain rights in the invention.

### TECHNICAL FIELD

The invention is directed to bisphenol compounds that are useful as antibacterials and bacteriostats.

### BACKGROUND

In the past decade, studies have correlated that healthy human microbiomes play a role in combating “modern plagues” such as diabetes, obesity, Crohn's, and celiac disease. The oral microbiome, a specific microbial niche residing in the oral cavity of humans, has gained attention due to the complexity in which the microorganisms interact both in commensal and pathogenic manners. This ecosystem is home to various species of bacteria that have adapted to live in the microaerophilic and/or anaerobic environments that exist surrounding the teeth, gingiva, and tongue.

Scientists have turned to chemical biology to develop new tools to study these systems and understand them at the molecular level. Interest in S. mutans has grown since it has been highly associated with the formation of carries via environmental acidification which leads to the corrosion of tooth enamel. Early colonizers such as commensal Streptococcus sanguinis and Streptococcus gordonii are responsible for early plaque formation by anchoring adhesion proteins to the pellicle of the tooth and producing glucan polymers that constitute the matrix of dental plaque. S. mutans is able to invade this matrix, form microcolonies, and eventually develop into a mature biofilm that is responsible for tooth decay via acidification. Another lesser known and more harrowing disease that has been associated with S. mutans biofilm growth is infective endocarditis, or inflammation of the inner tissues of the heart. S. mutans has the capability to nest itself in the heart as a mature biofilm and block the blood supply to the inner heart tissues causing inflammation. To date, few natural products have been reported to be effective inhibitors of the oral pathogen S. mutans.

Biofilms are of particular importance to the oral cavity as they protect the bacteria and allow them to outcompete other colonizers by decreasing the local pH, thereby causing enamel erosion and other pathologies resulting from the acidified environment.

The natural product carolacton has recently been the focus of attention. Carolacton specifically targets S. mutans cells as they transition into a biofilm. In contrast, the phenolic natural products honokiol has received attention due to the reportedly potent inhibitory activity against S. mutans. Although isolated and first reported in 1982 from the bark or seeds of a Magnolia tree, honokiol has been used as therapeutics in Chinese, Japanese and Korean traditional herbal remedies for centuries.

Accordingly, there is a long-felt need in the art for alternate inhibitors of oral pathogens.

### SUMMARY

In some aspects, compounds of formula (I) or salts thereof are provided, wherein R¹-R¹⁰ and x are defined herein.

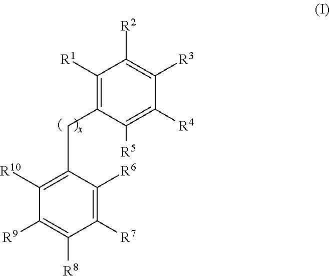

In further aspects, compounds of formula (IIA) of salts thereof are provided where R′, R², R⁴-R⁷, R⁹, R¹⁰, and x are defined herein.

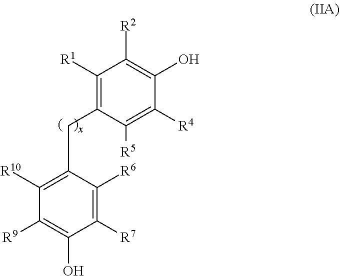

In other aspects, compounds of formula (IIB) or salts thereof are provided, wherein R¹-R⁴, R⁷-R¹⁰ and x are defined herein.

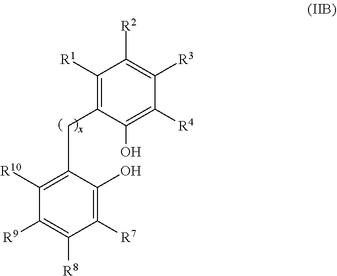

In yet further aspects, compounds of formula (IIC) or salts thereof are provided, wherein R¹, R², R⁴, R⁵, R⁷-R^{10′} and x are defined herein:

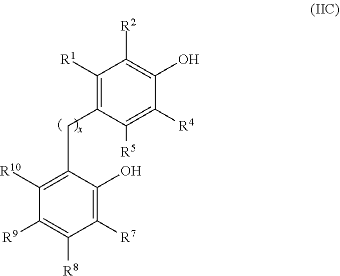

In yet other aspects, compounds of formula (III) or salts thereof are provided, wherein R¹, R², R⁴, R⁵, R⁷-R^{10′}, and x are defined herein.

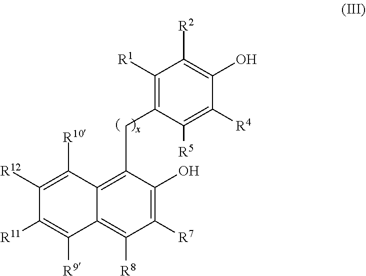

In other aspects, the disclosure provides compounds of formula (IV) or salts thereof, wherein R², R³, R⁷, and R⁸ are defined herein and n is 0-5.

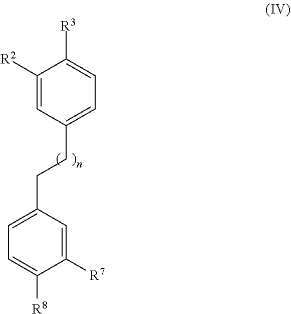

In yet further aspects, compounds of formula (V) or pharmaceutically acceptable salts thereof are provided, wherein x, R¹ to R³ and R⁶ to R⁸ are defined herein. In some embodiments, at least one of R¹ to R³ or R⁶ to R⁸ is OH.

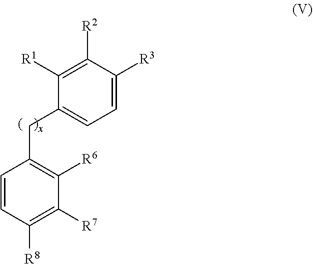

In further aspects, compositions are provided comprising one or more compounds described herein and an excipient.

In other aspects, methods of inhibiting, reducing, or ameliorating bacterial growth on a substrate are provided and comprise contacting the substrate with a compound described herein.

In still further aspects, dental care products, soaps, antibacterial products, and plastics are provided and comprise one or more compounds described herein.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific compositions, methods, and compositions disclosed. In addition, the drawings are not necessarily drawn to scale.

FIGS. 1A-1F are line graphs showing the effect of certain compounds on cell permeabilization at varying concentration. FIG. 1A shows the effects of compound 1H (data points for the 8 and 4 μM are overlapping with the 2 μM data points), FIG. 1B shows the effects of compound 3N (data points for the 8 and 4 μM are overlapping with the 2 μM data points), FIG. 1C shows the effects of compound 2E (data points for the 63, 32, 16, 8 and 4 μM are overlapping with the 2 μM data points), FIG. 1D shows the effects of negative control vancomycin (data points for the 63, 32, 16, 8 and 4 μM are overlapping with the 2 μM data points), FIG. 1D shows the effects of positive control QAC, and FIG. 1F shows the treated cells alone (data points for the 63, 32, 16, 8 and 4 μM are overlapping with the 2 μM points).

### DETAILED DESCRIPTION OF ILLUSTRATIVE EMBODIMENTS

In pursuing efforts to find compounds having antibacterial and/or bacteriostatial activity, the inventors discovered small molecules having higher potency than other antibacterials in the art. In addition, these compounds have antibacterial or bacteriostatial activity against a wide range of bacteria.

In the present disclosure the singular forms “a”, “an” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” or “substantially” it will be understood that the particular value forms another embodiment. In general, use of the term “about” or “substantially” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about” or “substantially”. In other cases, the gradations used in a series of values may be used to determine the intended range available to the term “about” or “substantially” for each value. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

When a range of carbon atoms is used herein, for example, C₁₋₆, all ranges, as well as individual numbers of carbon atoms are encompassed. For example, “C₁₋₃” includes C₁₋₃, C₁₋₂, C₂₋₃, C₁, C₂, and C₃.

As used herein, the term “compound(s) of formula (I)” includes those compounds of “formula (I),” as well as compounds of any of the formula (I) subgenera. The term “compound(s) of formula (II)” includes those compounds of “formula (II),” as well as compounds of any of the formula (II) subgenera. The term “compound(s) of formula (III)” includes those compounds of “formula (III),” as well as compounds of any of the formula (III) subgenera. The term “compound(s) of formula (IV)” includes those compounds of “formula (IV),” as well as compounds of any of the formula (IV) subgenera. The term “compound(s) of formula (V)” includes those compounds of “formula (V),” as well as compounds of any of the formula (V) subgenera.

“Salt” refers to a salt of a compound of the disclosure that possesses the desired activity of the parent compound. In some embodiments, the salt is a pharmaceutically acceptable salt. In other embodiments, the salts are non-toxic The salts may be inorganic or organic acid addition salts and base addition salts. Specifically, the salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like. When the compound contains a basic functionality, salts of non-toxic organic or inorganic acids, such as hydrochloride, hydrobromide, tartrate, mesylate, acetate, maleate, oxalate and the like.

“Subject” includes a mammal or non-mammal. In some embodiments, the subject is a mammal including, but not limited to, a human; a non-human primate; a rodent such as a mouse, rat, or guinea pig; a domesticated pet such as a cat or dog; a horse, cow, pig, sheep, goat, or rabbit. In other embodiments, the subject is non-mammalian including, but not limited to, a bird such as a duck, goose, chicken, or turkey. In further embodiment, subjects can be either gender and can be any age. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base.

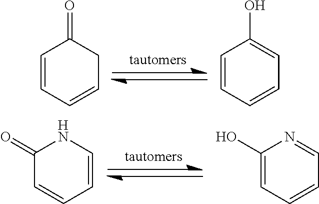

Another example of tautomerism is the acid- and nitro-forms of phenyl nitromethane, that are likewise formed by treatment with acid or base. Tautomeric forms may be relevant to the attainment of the optimal chemical reactivity and biological activity of a compound of interest.

Within the present disclosure, any open valency appearing on a carbon, oxygen, or nitrogen atom in any structure described herein indicates the presence of a hydrogen atom. Where a chiral center exists in a structure, but no specific stereochemistry is shown for that center, both enantiomers, separately or as a mixture, are encompassed by that structure. The methods for the determination of stereochemistry and the separation of stereoisomers are well-known in the art.

The term “alkyl,” when used alone or as part of a substituent group, refers to a straight- or branched-chain alkyl group having from 1 to 12 carbon atoms (“C₁₋₁₂”), preferably 1 to 8 carbon atoms (“C₁₋₈”), or preferably 1 to 6 carbons atoms (“C₁₋₆”), in the chain. Examples of alkyl groups include methyl (Me, C₁alkyl) ethyl (Et, C₁alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₁alkyl), isopentyl (C₁alkyl), tert-pentyl (C₁alkyl), hexyl (C₆alkyl), isohexyl (C₆alkyl), and groups that in light of the ordinary skill in the art and the teachings provided herein would be considered equivalent to any one of the foregoing examples.

The term “alkenyl,” when used alone or as part of a substituent group, refers to a straight- or branched-chain alkenyl group comprising at least one double bond. The alkenyl group has from 2 to 12 carbon atoms (“C₂₋₁₂”), preferably 2 to 6 carbons atoms (“C₂₋₆”), in the chain. The alkenyl may have one, two, three, or four double bonds, as permitted by the valency of the substituents. Examples of alkenyl groups include ethenyl (C₂alkenyl), n-propenyl (C₃alkenyl), isopropenyl (C₃alkenyl), butenyl (C₄alkenyl), isobutenyl (C₄alkenyl), sec-butenyl (C₄alkenyl), pentenyl (C₅alkenyl), isopentenyl (C₅alkenyl), tert-pentenyl (C₁alkyl), hexenyl (C₆alkenyl), isohexenyl (C₆alkenyl), and groups that in light of the ordinary skill in the art and the teachings provided herein would be considered equivalent to any one of the foregoing examples. An alkyl may be optionally substituted with one, two, or three substituents selected from halo (F, Cl, Br, or I, preferably F), —OH, —OC₁-C₆alkyl, C₂₋₆alkenyl, C₂₋₆alkynyl, —CN, —NH₂, —NH(C₁-C₆alkyl), —NH(C₁-C₆alkyl)NH₂, —NH(C₁-C₆alkyl)NHC(O)OC₁₋₆alkyl, —NH(C₁-C₆alkyl)₂, aryl, or heteroaryl.

The term “alkoxy” as used herein refers to the 0-(alkyl) group, where the point of attachment is through the oxygen-atom and the alkyl group is defined above. An alkoxy may be optionally substituted with one, two, or three substituents selected from halo (F, Cl, Br, or I, preferably F), —OH, —OC₁-C₆alkyl, —CN, —NH₂, —NH(C₁-C₆alkyl), —NH(C₁-C₆alkyl)₂, aryl, or heteroaryl.

The term “cycloalkyl” refers to a cyclic aliphatic having 3 to 10 carbon atoms, preferably 3 to 8 carbon atoms, e.g., 3, 4, 5, 6, 7, or 8 carbon atoms and includes, for example, cyclopropyl, cyclobutyl, cyclopentyl, cyclohexyl, cycloheptyl, or cyclooctyl. A cycloalkyl may be optionally substituted with one, two, or three substituents selected from halo (F, Cl, Br, or I, preferably F), —OH, C₁-C₆alkyl, —OC₁-C₆alkyl, —CN, —NH₂, —NH(C₁-C₆alkyl), —NH(C₁-C₆alkyl)₂, aryl, or heteroaryl.

The term “halogen” or “halo” represents chlorine, fluorine, bromine, or iodine.

The term “aryl” refers to 6-15 membered monoradical bicyclic or tricyclic hydrocarbon ring systems, including bridged, spiro, and/or fused ring systems, in which at least one of the rings is aromatic. An aryl group may contain 6 or about 9 to about 15 ring atoms, such as 6 (i.e., phenyl) or about 9 to about 11 ring atoms. In certain embodiments, aryl groups include, but are not limited to, naphthyl, indanyl, indenyl, anthryl, phenanthryl, fluorenyl, 1,2,3,4-tetrahydronaphthalenyl, 6,7,8,9-tetrahydro-5H-benzocycloheptenyl, and 6,7,8,9-tetrahydro-5H-benzocycloheptenyl. In some embodiments, the aryl is phenyl or napthyl. An aryl may be optionally substituted with one, two, or three substituents that are halo (F, Cl, Br, or I), —OH, C₁₋₆alkyl, C₂₋₆alkenyl, C₃₋₈ cycloalkyl, —OC₁₋₆alkyl, —CN, —NH₂, —NH(C₁-C₆alkyl), —NH(C₁-C₆alkyl)₂, aryl or heteroaryl.

The term “heteroaryl” refers to (a) 5 and 6 membered monocyclic aromatic rings, which contain, in addition to carbon atoms, at least one heteroatom, such as nitrogen, oxygen or sulfur, and (b) 7-15 membered bicyclic and tricyclic rings, which contain, in addition to carbon atoms, at least one heteroatom, such as nitrogen, oxygen or sulfur, and in which at least one ring is aromatic. Heteroaryl groups can be bridged, spiro, and/or fused. In further embodiments, a heteroaryl may contain 5 to about 15 ring atoms. In further embodiments, a heteroaryl may contain 5 to about 10 ring atoms, such as 5, 6, 9, or 10 ring atoms. The heteroaryl may be C-attached or N-attached where such is possible and results in the creation of a stable structure. Examples include, but are not limited to 2,3-dihydrobenzofuranyl, 1,2-dihydroquinolinyl, 3,4-dihydroisoquinolinyl, 1,2,3,4-tetrahydroisoquinolinyl, 1,2,3,4-tetrahydroquinolinyl, benzoxazinyl, benzthiazinyl, chromanyl, furanyl, imidazolyl, isoxazolyl, isothiazolyl, oxadiazolyl, oxazolyl, pyridinyl, pyrimidinyl, pyrazolyl, pyrrolyl, pyrazinyl, pyridazinyl, pyrazinyl, thienyl, tetrazolyl, thiazolyl, thiadiazolyl, triazinyl, triazolyl, naphthyridinyl, pteridinyl, phthalazinyl, purinyl, alloxazinyl, benzimidazolyl, benzofuranyl, benzofurazanyl, 2H-1-benzopyranyl, benzothiadiazinyl, benzothiazinyl, benzothiazolyl, benzothiophenyl, benzoxazolyl, benzoxadiazolyl such as benzo[c][1,2,5]oxadiazolyl, cinnolinyl, furopyridinyl, indolinyl, indolizinyl, indolyl, quinazolinyl, quinoxalinyl, isoindolyl, isoquinolinyl, 10-aza-tricyclo[6.3.1.0^{2,7}]dodeca-2(7),3,5-trienyl, 12-oxa-10-aza-tricyclo[6.3.1.0^{2,7}]dodeca-2(7),3,5-trienyl, 12-aza-tricyclo[7.2.1.0^{2,7}]dodeca-2(7),3,5-trienyl, 10-aza-tricyclo[6.3.2.0^{2,7}]trideca-2(7),3,5-trienyl, 2,3,4,5-tetrahydro-1H-benzo[d]azepinyl, 1,3,4,5-tetrahydro-benzo[d]azepin-2-onyl, 1,3,4,5-tetrahydro-benzo[b]azepin-2-onyl, 2,3,4,5-tetrahydro-benzo[c]azepin-1-onyl, 1,2,3,4-tetrahydro-benzo[e][1,4]diazepin-5-onyl, 2,3,4,5-tetrahydro-1H-benzo[e][1,4]diazepinyl, 5,6,8,9-tetrahydro-7-oxa-benzocycloheptenyl, 2,3,4,5-tetrahydro-1H-benzo[b]azepinyl, 1,2,4,5-tetrahydro-benzo[e][1,3]diazepin-3-onyl, 3,4-dihydro-2H-benzo[b][1,4]dioxepinyl, 3,4-dihydro-2H-benzo[f][1,4]oxazepin-5-onyl, 6,7,8,9-tetrahydro-5-thia-8-aza-benzocycloheptenyl, 5,5-dioxo-6,7,8,9-tetrahydro-5-thia-8-aza-benzocycloheptenyl, and 2,3,4,5-tetrahydro-benzo[f][1,4]oxazepinyl. A heteroaryl may be optionally substituted with one, two, or three substituents that are halo (F, Cl, Br, or I), —OH, C₁₋₆alkyl, C₂₋₆alkenyl, C₃₋₈cycloalkyl, —OC₁₋₆alkyl, —CN, —NH₂, —NH(C₁-C₆alkyl), —NH(C₁-C₆alkyl)₂, aryl or heteroaryl.

### I. THE COMPOUNDS

In some embodiments, the compound is of formula (I):

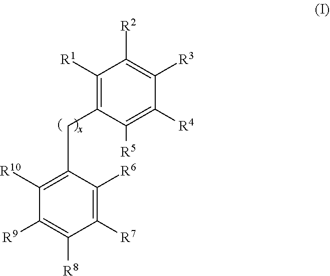

In these compounds, R¹ and R⁵ are, independently, H, OH, C₁₋₆alkyl, or C₃₋₈cycloalkyl. In some embodiments, R¹ is H. In other embodiments, R¹ is OH. In further embodiments, R¹ is C₁₋₆alkyl. In other embodiments, R¹ is methyl. In yet other embodiments, R⁵ is H. In still further embodiments, R⁵ is OH. In other embodiments, R⁵ is C₁₋₆alkyl. In yet other embodiments, R⁵ is methyl. In further embodiments, R¹ and R⁵ are H. In still other embodiments, R¹ and R⁵ are OH. In yet further embodiments, R¹ and R⁵ are C₁₋₆alkyl. In other embodiments, R¹ and R⁵ are methyl.

R² and R⁴ are, independently, H, OH, C₁₋₆alkyl, C₂₋₆alkenyl, C₁₋₆alkoxy, or C₃₋₈cycloalkyl. In some embodiments, R² is H. In other embodiments, R² is C₁₋₆alkyl, i.e., methyl, n-propyl, i-propyl, or t-butyl. In still other embodiments, R² is C₂₋₆alkenyl, i.e., ethenyl, propenyl, butenyl, pentenyl, or hexenyl. In other embodiments, R² is allyl. In still further embodiments, R² is C₁₋₆alkoxy, i.e., methoxy, ethoxy, propoxy, butoxy, pentoxy, or hexoxy. In further embodiments, R² is methoxy. In other embodiments, R² is OH. In further embodiments, R⁴ is H. In other embodiments, R⁴ is C₁₋₆alkyl, i.e., methyl, n-propyl, i-propyl, or t-butyl. In still other embodiments, R⁴ is C₂₋₆alkenyl, i.e., ethenyl, propenyl, butenyl, pentenyl, or hexenyl. In other embodiments, R⁴ is allyl. In still further embodiments, R⁴ is C₁₋₆alkoxy, i.e., methoxy, ethoxy, propoxy, butoxy, pentoxy, or hexoxy. In further embodiments, R⁴ is methoxy. In other embodiments, R⁴ is OH. In other embodiments, R² and R⁴ are OH. In still further embodiments, R² and R⁴ are H. In still further embodiments, R² and R⁴ are C₁₋₆alkyl, i.e., methyl, ethyl, propyl, butyl, pentyl, or hexyl. In other embodiments, R² and R⁴ are C₂₋₆alkenyl, i.e., ethenyl, propenyl, butenyl, pentenyl, or hexenyl. In still other embodiments, R² and R⁴ are allyl. In further embodiments, R² and R⁴ are C₁₋₆alkoxy, i.e., methoxy, ethoxy, propoxy, butoxy, pentoxy, or hexoxy. In other embodiments, R² and R⁴ are methoxy.

R³ and R⁸ are, independently, H, OH, C₁₋₆alkyl, C₃₋₈cycloalkyl, or optionally substituted aryl. In some embodiments, R³ is C₁₋₆alkyl, i.e., methyl, ethyl, propyl, butyl, pentyl, or hexyl. In other embodiments, R³ is methyl or t-butyl. In further embodiments, R³ is OH. In still other embodiments, R³ is H. In yet further embodiments, R³ is optionally substituted aryl. In other embodiments, R³ is optionally substituted phenyl. In further embodiments, R⁸ is C₁₋₆ alkyl, i.e., methyl, ethyl, propyl, butyl, pentyl, or hexyl. In still other embodiments, R⁸ is methyl or t-butyl. In yet further embodiments, R⁸ is OH. In other embodiments, R⁸ is H. In further embodiments, R⁸ is optionally substituted aryl. In yet other embodiments, R⁸ is optionally substituted phenyl. In further embodiments, R³ and R⁸ are C₁₋₆alkyl, i.e., methyl, ethyl, propyl, butyl, pentyl, or hexyl. In other embodiments, R³ and R⁸ are methyl or t-butyl. In still further embodiments, R³ and R⁸ are OH. In yet other embodiments, R³ and R⁸ are H. In further embodiments, R³ and R⁸ are optionally substituted aryl. In other embodiments, R³ and R⁸ are optionally substituted phenyl.

R⁶ and R¹⁰ are, independently, H, OH, C₁₋₆alkyl, or C₃₋₈cycloalkyl. In some embodiments, R⁶ is H. In further embodiments, R⁶ is OH. In other embodiments, R⁶ is C₁₋₆alkyl, i.e., methyl, ethyl, propyl, butyl, pentyl, or hexyl. In still further embodiments, R⁶ is methyl or i-propyl. In some embodiments, R¹⁰ is H. In other embodiments, R¹⁰ is OH. In further embodiments, R¹⁰ is C₁₋₆alkyl, i.e., methyl, ethyl, propyl, butyl, pentyl, or hexyl. In still other embodiments, R¹⁰ is methyl or i-propyl. In some embodiments, R⁶ and R¹⁰ are H. In further embodiments, R⁶ and R¹⁰ are OH. In other embodiments, R⁶ and R¹⁰ are C₁₋₆alkyl, i.e., methyl, ethyl, propyl, butyl, pentyl, or hexyl. In still further embodiments, R⁶ and R¹⁰ are methyl or i-propyl.

R⁷ and R⁹ are, independently, H, OH, C₁₋₆alkyl, C₂₋₆alkenyl, C₃₋₈cycloalkyl, or optionally substituted aryl. In some embodiments, R⁷ is H. In other embodiments, R⁷ is OH. In further embodiments, R⁷ is C₁₋₆alkyl, i.e., methyl, ethyl, propyl, butyl, pentyl, or hexyl. In still other embodiments, R⁷ is methyl or n-propyl. In yet further embodiments, R⁷ is C₂₋₆alkenyl, i.e., ethenyl, propenyl, butenyl, pentenyl, or hexenyl. In other embodiments, R⁷ is propenyl. In further embodiments, R⁷ is optionally substituted aryl. In yet other embodiments, R⁷ is optionally substituted phenyl. In some embodiments, R⁹ is H. In other embodiments, R⁹ is OH. In further embodiments, R⁹ is C₁₋₆alkyl, i.e., methyl, ethyl, propyl, butyl, pentyl, or hexyl. In still other embodiments, R⁹ is methyl or n-propyl. In yet further embodiments, R⁹ is C₂₋₆alkenyl, i.e., ethenyl, propenyl, butenyl, pentenyl, or hexenyl. In other embodiments, R⁹ is propenyl. In further embodiments, R⁹ is optionally substituted aryl. In yet other embodiments, R⁹ is optionally substituted phenyl. In some embodiments, R⁷ and R⁹ are H. In other embodiments, R⁷ and R⁹ are OH. In further embodiments, R⁷ and R⁹ are C₁₋₆alkyl, i.e., methyl, ethyl, propyl, butyl, pentyl, or hexyl. In still other embodiments, R⁷ and R⁹ are methyl or n-propyl. In yet further embodiments, R⁷ and R⁹ are C₂₋₆alkenyl, i.e., ethenyl, propenyl, butenyl, pentenyl, or hexenyl. In other embodiments, R⁷ and R⁹ are propenyl. In further embodiments, R⁷ and R⁹ are optionally substituted aryl. In yet other embodiments, R⁷ and R⁹ are optionally substituted phenyl.

Alternatively, R⁹ and R¹⁰ are joined to form —C(R^{9′})═C(R¹¹)—C(R¹²)—C(R^{10′})—. In some embodiments, R^{9′}, R^{10′}, R¹¹ and R¹² are, independently, H, OH, halogen, C₁₋₆alkyl, C₁₋₆alkoxy, C₃₋₈cycloalkyl, C(O)(C₁₋₆alkyl), optionally substituted aryl, or optionally substituted heteroaryl. In some embodiments, R^{9′} is H. In other embodiments, R^{9′} is OH. R^{9′} is halogen. R^{9′} is heteroaryl. R^{9′} is C₁₋₆alkoxy. R^{9′} is C(O)C₁₋₆alkoxy, such as C(O)OCH₃. In some embodiments, R^{10′} is H. In yet other embodiments, R^{10′} is OH. R^{10′} is halogen. R^{10′} is heteroaryl. R^{10′} is C₁₋₆alkoxy. R^{10′} is C(O)C₁₋₆alkoxy. In some embodiments, R^{9′} and R^{10′} are H. In some embodiments, R¹¹ is H. R¹¹ is OH. R¹¹ is halogen. R¹¹ is heteroaryl. R¹¹ is C₁₋₆alkoxy. R¹¹ is C(O)C₁₋₆alkoxy. In some embodiments, R¹² is H. R¹² is OH. R¹² is halogen. R¹² is C₁₋₆alkoxy. R¹² is heteroaryl. R¹² is C(O)C₁₋₆alkoxy, such as C(O)OCH₃. In some embodiments, R¹¹ and R¹² are H. In other embodiments, R^{9′}, R^{10′}, R¹¹, or R¹² is halogen. In further embodiments, R^{9′}, R^{10′}, R¹¹, or R¹² is bromo. In other embodiments, R^{9′}, R^{10′}, R¹¹, or R¹² is C₁₋₆alkoxy. In still further embodiments, R^{9′}, R^{10′}, R¹¹, or R¹² is methoxy. R^{9′}, R^{10′}, R¹¹, or R¹² is heteroaryl. In yet other embodiments, R^{9′}, R^{10′}, R¹¹, or R¹² is pyrazolyl. In further embodiments, one or more of R^{9′}, R^{10′}, R¹¹, or R¹² is C(O)C₁₋₆alkoxy. In other embodiments, one or more of R^{9′}, R^{10′}, R¹¹, or R¹² is C(O)OCH₃.

In these compounds, x is 0 to 8. In some embodiments, x is 0 to 6. In other embodiments, x is 0 to 4. In further embodiments, x is 0 to 2. In still other embodiments, x is 0. In yet further embodiments, x is 1. In other embodiments, x is 2. In further embodiments, x is 3. In still other embodiments, x is 4. In yet further embodiments, x is 5. In yet other embodiments, x is 6. In further embodiments, x is 7. In other embodiments, x is 8.

In these compounds and in some embodiments, at least one of R¹ to R⁵ is OH. In further embodiments, at least one of R⁶ to 10° is OH. In other embodiments, at least one of R¹ to R⁵ is OH and at least one of R⁶ to R^{10′} is OH.

In some embodiments, the compound of formula (I) is not (i) [1,1′-biphenyl]-2,2′-diol, (ii) [1,1′-biphenyl]-2,4′-diol, (iii) [1,1′-biphenyl]-3,3′-diol, (iv) [1,1′-biphenyl]-4,4′-diol, (v) 3,3′,6,6′-tetramethyl-[1,1′-biphenyl]-2,2′-diol, (vi) (vii) 3,3′,4,4′,6,6′-hexamethyl-[1,1′-biphenyl]-2,2′-diol, (viii) (ix) 3,3′-diallyl-4,4′,6,6′-tetramethyl-[1,1′-biphenyl]-2,2′-diol, (x) 4,4′,6,6′-tetramethyl-3,3′-dipropyl-[1,1′-biphenyl]-2,2′-diol, (xi) 4,4′-dimethoxy-3,3′,6,6′-tetramethyl-[1,1′-biphenyl]-2,2′-diol, (xii) [1,1′-biphenyl]-3,3′,4-triol, (xiii) [1,1′-biphenyl]-3,3′,4′,5′-tetraol, (xiv) 3,3′,6,6′-tetramethyl-[1,1′-biphenyl]-2,2′,4,4′-tetraol, (xv) 4′,5″-dimethyl-[1,1′:2′,1″:2″,1′″-quaterphenyl]-3′,4″,5′,6″-tetraol, or (xvi) 3,3′-dimethyl-6,6′-di(oct-1-yn-1-yl)-[1,1′-biphenyl]-2,2′,4,4′-tetraol. In further embodiments, the compound of formula (I) is not 3′,5-diallyl-[1,1′-biphenyl]-2,4′-diol.

In some aspects, the compound is of formula (IIA) or a salt thereof, wherein R¹, R², R⁴-R⁷, R⁹, R¹⁰, and x are defined above. In some embodiments, x is 0.

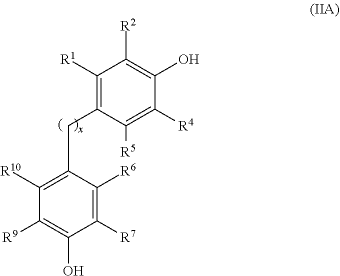

In other aspects, the compound is of formula (JIB) or a salt thereof, wherein R¹-R⁴, R⁷-R¹⁰ and x are defined herein. In some embodiments, x is 0. In other embodiments, R² and R⁴ are C₁₋₆alkyl. In further embodiments, R² is C₁₋₆alkyl and R⁴ is C₂₋₆alkenyl. In yet other embodiments, R² and R⁴ are C₁₋₆alkoxy. In further embodiments, R¹ and R⁵ are H and/or R⁷ and R⁸ are H and/or R^{9′}, R^{10′}, R¹¹, and R¹² are H.

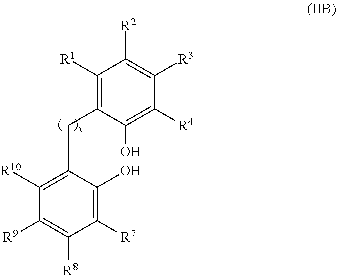

In further aspects, the compound is of formula (IIC) or a salt thereof, wherein R¹, R², R⁴, R⁵, R⁷-R¹⁰, and x are defined herein. In other embodiments, R² and R⁴ are C₁₋₆alkyl. In further embodiments, R² is C₁₋₆alkyl and R⁴ is C₂₋₆alkenyl. In yet other embodiments, R² and R⁴ are C₁₋₆alkoxy. In further embodiments, R¹ and R⁵ are H and/or R⁷ and R⁸ are H and/or R^{9′}, R^{10′}, R¹¹, and R¹² are H.

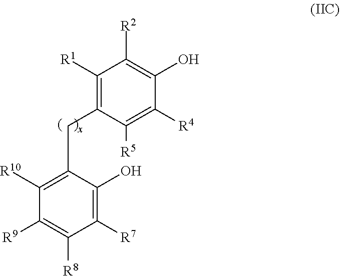

In yet other aspects, the compound is of formula (III) or a salt thereof, wherein R¹, R², R⁴, R⁵, R⁷, R⁸, R^{9′}, R^{10′}, R¹¹, R¹², and x are defined herein. In some embodiments, x is 0. In other embodiments, R² and R⁴ are C₁₋₆alkyl. In further embodiments, R² is C₁₋₆alkyl and R⁴ is C₂₋₆alkenyl. In yet other embodiments, R² and R⁴ are C₁₋₆alkoxy. In further embodiments, R¹ and R⁵ are H and/or R⁷ and R⁸ are H and/or R^{9′}, R^{10′}, R¹¹, and R¹² are H.

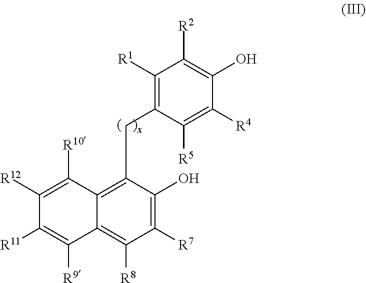

In yet other aspects, the compound is of formula (IV) or a salt thereof, wherein R², R³, R⁷, and R⁸ are defined herein and n is 0-5. In some embodiments, n is 0. In other embodiments, n is 1 or 2.

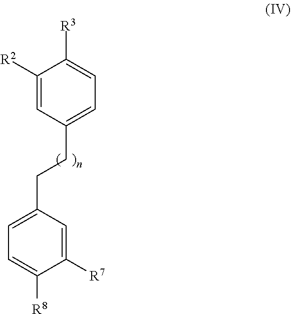

In further aspects, the compound is of formula (V) or a pharmaceutically acceptable salt thereof, wherein x, R¹ to R³ and R⁶ to R⁸ are defined herein and with the proviso that at least one of R¹ to R³ or R⁶ to R⁸ is OH. n some embodiments, R¹ is H, OH, or C₁₋₆alkyl. In other embodiments, R¹ is H. In further embodiments, R¹ is C₁₋₆alkyl such as ^{t}Bu. In still further embodiments, R¹ is OH. In some embodiments, R² is H, OH, C₁₋₆alkyl, or C₁₋₆alkoxy. In other embodiments, R² is OH. In further embodiments, R² is C₁₋₆alkyl such as ^{t}Bu. In still other embodiments, R² is C₁₋₆alkoxy such as methoxy, ethoxy, propoxy, butoxy, pentoxy, or hexoxy, preferably methoxy, ethoxy, or propoxy, even more preferably, methoxy or ethoxy, and still more preferably methoxy. In some embodiments, R³ is H, OH, C₁₋₈alkyl, or C₃₋₈cycloalkyl. In other embodiments, R³ is C₁₋₆alkyl such as ^{t}Bu. In some embodiments, R⁶ is H, OH, or C₁₋₆alkyl. In other embodiments, R⁶ is OH. In further embodiments R⁶ is C₁₋₆alkyl such as ^{t}Bu. In some embodiments, R⁷ is H, OH, or C₁₋₆alkyl. In other embodiments, R⁷ is OH. In further embodiments, R⁷ is C₁₋₆alkyl. In still other embodiments, R⁷ is ^{t}Bu. In some embodiments, R⁸ is H, OH, C₁₋₈alkyl, or C₃₋₈cycloalkyl. In other embodiments, R⁸ is H. In further embodiments, R⁸ is OH. In yet other embodiments, R⁸ is C₁₋₆alkyl such as ^{t}Bu. In still further embodiments, R⁸ is C₁₋₆alkoxy such as methoxy, ethoxy, propoxy, butoxy, pentoxy, or hexoxy, preferably methoxy, ethoxy, or propoxy, even more preferably, methoxy or ethoxy, and still more preferably methoxy. In some embodiments, x is 0 to 8. In other embodiments, x is 2 to 6 such as 2. In some preferred embodiments, R¹, R³, and R⁶ are H. In other preferred embodiments, R¹ and R⁶ are H. In further preferred embodiments, R³ and R⁸ are H. In still other preferred embodiments, one of R¹ to R³ or R⁶ to R⁸ is ^{t}Bu. In some embodiments, one of R¹ to R³ or R⁶ to R⁸ is OH. In other embodiments, one of R¹ to R³ is OH and one of R⁶ to R⁸ is OH.

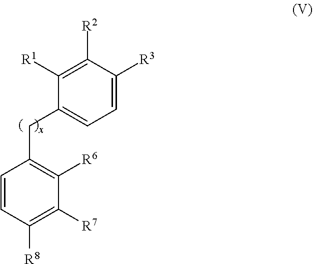

In other aspects, the compound is of formula (VA), wherein x, R², R³, R⁷, and R⁸ are defined herein. In some embodiments, R² is H, OH, C₁₋₆alkyl, or C₁₋₆alkoxy. In other embodiments, R² is OH. In further embodiments, R² is C₁₋₆alkyl such as ^{t}Bu. In still other embodiments, R² is C₁₋₆alkoxy such as methoxy, ethoxy, propoxy, butoxy, pentoxy, or hexoxy, preferably methoxy, ethoxy, or propoxy, even more preferably, methoxy or ethoxy, and still more preferably methoxy. In some embodiments, R³ is H, OH, C₁₋₆alkyl, or C₃₋₈cycloalkyl. In other embodiments, R³ is C₁₋₆alkyl such as ^{t}Bu. In some embodiments, R⁷ is H, OH, or C₁₋₆alkyl. In other embodiments, R⁷ is OH. In further embodiments, R⁷ is C₁₋₆alkyl. In still other embodiments, R⁷ is ^{t}Bu. In some embodiments, R⁸ is H, OH, C₁₋₆alkyl, or C₃₋₈cycloalkyl. In other embodiments, R⁸ is H. In further embodiments, R⁸ is OH. In yet other embodiments, R⁸ is C₁₋₆alkyl such as ^{t}Bu. In still further embodiments, R⁸ is C₁₋₆alkoxy such as methoxy, ethoxy, propoxy, butoxy, pentoxy, or hexoxy, preferably methoxy, ethoxy, or propoxy, even more preferably, methoxy or ethoxy, and still more preferably methoxy. In some embodiments x is 2 to 6, preferably 2 to 4, or more preferably 2.

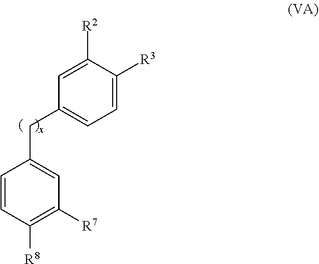

In further aspects, the compound is of formula (VB), wherein x, R³ and R⁸ are defined herein. In some embodiments, R³ is H, OH, C₁₋₆alkyl, or C₃₋₈cycloalkyl. In other embodiments, R³ is C₁₋₆alkyl such as ^{t}Bu. In further embodiments, R³ is OH. In some embodiments, R⁸ is H, OH, C₁₋₆alkyl, or C₃₋₈cycloalkyl. In other embodiments, R⁸ is H. In further embodiments, R⁸ is OH. In yet other embodiments, R⁸ is C₁₋₆alkyl such as ^{t}Bu. In still further embodiments, R⁸ is C₁₋₆alkoxy such as methoxy, ethoxy, propoxy, butoxy, pentoxy, or hexoxy, preferably methoxy, ethoxy, or propoxy, even more preferably, methoxy or ethoxy, and still more preferably methoxy. In some embodiments x is 2 to 6, preferably 2 to 4, or more preferably 2.

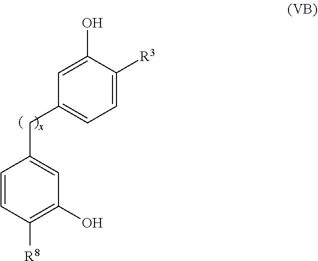

### II. COMPOSITIONS

The compounds of the disclosure are used, alone or in combination with one or more additional ingredients, to formulate compositions of the disclosure. A composition of the disclosure comprises: (a) an effective amount of at least one compound in accordance with the disclosure; and (b) an excipient. In some embodiments, the composition contains a compound of formula (I), or a salt thereof. In other embodiments, the composition contains a compound of formula (IIA), or a salt thereof. In further embodiments, the composition contains a compound of formula (IIB), or a salt thereof. In additional embodiments, the composition contains a compound of formula (IIC), or a salt thereof. In further embodiments, the composition contains a compound of formula (III), or a salt thereof. In still further embodiments, the composition contains a compound of formula (IV), or a salt thereof. In further embodiments, the composition contains a compound of formula (V), or a salt thereof. In yet other embodiments, the composition contains more than one compound disclosed herein, for example, compounds of formula (I), (IIA), (IIB), (IIC), (III), (IV), (V) or salts thereof.

The compositions may also be formulated for administration to a subject and, thus, are “pharmaceutical compositions.” The compositions may be administered in the inventive methods by a suitable route of delivery, e.g., oral, parenteral, rectal, topical, or ocular routes, or by inhalation.

The composition may be in any form suitable for administration to a subject. In some embodiments, the pharmaceutical composition is in the form of tablets, capsules, sachets, lotions, dragees, powders, granules, lozenges, powders for reconstitution, liquid preparations, or suppositories. These delivery forms of the compositions containing one or more dosage units of the compounds discussed herein may be prepared using suitable excipients and compounding techniques known or that become available to those skilled in the art.

For oral administration, the compounds of the disclosure can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Oral tablets may include a compound according to the disclosure mixed with pharmaceutically acceptable excipients such as inert diluents, disintegrating agents, binding agents, lubricating agents, sweetening agents, flavoring agents, coloring agents, or preservative agents. Exemplary liquid oral excipients include ethanol, glycerol, water, and the like. In some embodiments, the liquid oral excipient is a dental care additive such as a toothpaste additive or mouth rinse.

Capsules for oral administration include hard and soft gelatin capsules. To prepare hard gelatin capsules, compounds of the disclosure may be mixed with a solid, semi-solid, or liquid diluent. Soft gelatin capsules may be prepared by mixing the compound of the disclosure with water, an oil such as peanut oil or olive oil, liquid paraffin, a mixture of mono and di-glycerides of short chain fatty acids, polyethylene glycol 400, or propylene glycol and an optional filler such as an inert diluent, disintegrating agent, binding agent, lubricating agent, sweetening agent, flavoring agent, coloring agent, or preservative agent. In some embodiments, the capsule comprises a dental care additive such as a toothpaste additive or mouth rinse.

Liquids for oral administration may be in the form of suspensions, solutions, emulsions or syrups or may be lyophilized or presented as a dry product for reconstitution with water or other suitable vehicle before use. Such liquid compositions may optionally contain pharmaceutically-acceptable excipients such as suspending agents, non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents; flavoring agents; coloring agents; disintegrating agent; or sweetening agent. In some embodiments, the liquid comprises a dental care additive such as a toothpaste additive or mouth rinse.

Compounds of the disclosure may alternatively be administered in methods of this disclosure by inhalation, via the nasal or oral routes, e.g., in a spray formulation also containing a suitable carrier.

The compounds of this disclosure may also be administered by non-oral routes. For example, the compositions may be formulated for rectal administration as a suppository. For parenteral use, including intravenous, intramuscular, intraperitoneal, or subcutaneous routes, the compounds of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical excipient such as ethanol, a soap, sterilization agent, among others.

Another mode of administering the compounds of the disclosure may utilize a patch formulation to affect transdermal delivery.

### III. METHODS

The compound and compositions described herein are useful in methods of inhibiting, reducing, or ameliorating bacterial growth on a substrate. Thus, the compounds are useful as antibacterials and/or bacteriostats.

The term “substrate” as used herein refers to a living subject as previously described or a non-living object.

The term “antibacterial” as used herein refers to a compound that reduces the amount of or eliminates the bacteria from a substrate. In some embodiments, the compound reduces the amount of bacteria from a substrate by at least about 50%. In other embodiments, the compound reduces the amount of bacteria from a substrate by at least about 60%. In further embodiments, the compound reduces the amount of bacteria from a substrate by at least about 70%. In yet embodiments, the compound reduces the amount of bacteria from a substrate by at least about 80%. In still further embodiments, the compound removes at least about 90%. In yet other embodiments, the compound reduces the amount of bacteria from a substrate by at least about 95%. In further embodiments, the compound reduces the amount of bacteria from a substrate by at least about 96, 97, 98, or 99%. In other embodiments, the compound eliminates, i.e., removes about 100%, of bacteria from a substrate.

The term “bacteriostat” as used herein refers to a compound that prevents the formation of bacteria on a substrate. In some embodiments, the compound keeps the surface at least about 50% free from bacteria. In other embodiments, the compound keeps the surface at least about 60% free from bacteria. In further embodiments, the compound keeps the surface at least about 70% free from bacteria. In yet embodiments, the compound keeps the surface at least about 80% free from bacteria. In still further embodiments, the compound keeps the surface at least about 90% free from bacteria. In yet other embodiments, the compound keeps the surface at least about 95% free from bacteria. In further embodiments, the compound keeps the surface at least about 96, 97, 98, or 99% free from bacteria. In other embodiments, the compound prevents bacterial formation from a surface, i.e., keep the surface 100% free of bacteria.

The compounds are effective against a wide range of bacteria and, thus, their activity may entail a broad-spectrum inhibitory mechanism. In some embodiments, the compounds are effective against pathogenic bacteria. The bacteria may be pathogenic to one or more parts of a subject, including, without limitation, bacteria that replicate externally or internally. In some embodiments, the bacteria grow on the skin, in the oral cavity, or in one or more tissue or organ in a subject. In other embodiments, the bacteria is gram positive. In further embodiments, the bacteria is gram negative. In other embodiments, the bacteria is a Streptococcal, Staphylococcus, Corynebacterium, Neisseria, Haemophilus, Treponema, Bartonella, Mycobacterium, Leptospira, Nocardia, Burkholderia, Actinomcyes, Brucella, Aeromonas, Fusibacterium, or Salmonella species such as S. typhi. In yet further embodiments, the bacteria is Bacillus fusiformis, Treponema vincenti, Erysipelothrix insidiosa, Helicobacter pylori, Klebsiella rhinoscleromatis, Mycoplasma, pneumonia, Pseudomonas aeruginosa, Calymmatobacterium granulomatis, Bacillus anthracis, Clostridium perfringens, Yersinia pestis, Serratia marcescens, or Vibrio vulnificus, among others. In yet other embodiments, the bacterium is a Streptococcal strain such as S. mutans, S. sanguinis, S. gordonii, or S. pyogenes. In yet other embodiments, the bacterium is a Staphylococcus strain such as S. aureus including methicillin resistant S. aureus. In further embodiments, the bacteria is a Salmonella, Campylobacter, E. coli, Listeria, or Clostridium bacteria. In still other embodiments, the bacteria is an oral bacterium such as, e.g., Rothia dentocariosa, Fusobacterium nucleatum, Prevotella oulorum, or combinations thereof.

The bacteria may be present on the substrate as a colony or in the form of a biofilm. The term “biofilm” as used herein refers to a group of bacteria which cells stick to each other. In some embodiments, the biofilm adheres to the substrate on which it forms. In other embodiments, the bacteria are in the form of a biofilm in an oral cavity.

In some methods described herein, a substrate is contacted with one or more compound of formula (I) or composition containing one or more compound described herein. The compound of formula (I) may be applied as determined by one skilled in the art. In some embodiments, the compound of formula (I) is poured, sprayed, or painted onto the substrate.

In treatment methods according to the disclosure, an effective amount of a compound according to the disclosure is administered to a subject. An “effective amount” means an amount or dose sufficient to generally bring about the desired a therapeutic benefit in subjects in need of such treatment. Effective amounts or doses of the compounds of the present disclosure may be ascertained by routine methods such as modeling, dose escalation studies or clinical trials, and by taking into consideration routine factors, e.g., the mode or route of administration or drug delivery, the pharmacokinetics of the compound, the severity and course of the infection, the subject's previous or ongoing therapy, the subject's health status and response to drugs, and the judgment of the treating physician. An example of a dose is in the range of from about 0.001 to about 200 mg of compound per kg of subject's body weight per day. For a 70-kg human, an illustrative range for a suitable dosage amount is from about 0.05 to about 7 g/day, or about 0.2 to about 2.5 g/day.

In addition, the compounds of the disclosure may be used in combination with additional ingredients in the treatment of the above conditions. The additional ingredients may be co-administered separately with a compound of the disclosure or included in a pharmaceutical composition according to the disclosure. In an exemplary embodiment, additional ingredients are those that are known or discovered to be effective in the treatment of any of the diseases or disorders described herein. In some embodiments, the additional agent is a second bacteriostat or antibacterial. The combination may serve to increase efficacy, decrease one or more side effects, or decrease the required dose of the compound according to the disclosure.

### IV. PRODUCTS/KITS

Also provided herein are kits or products containing one or more compounds of formula (I), (IIA), (IIB), (IIIC), (IV), and (V) or salts thereof, or compositions described herein. The compound or composition may also be sub-divided to contain appropriate quantities of one or more compound of formula (I), (IIA), (IIB), (IIIC), (IV), and (V). For example, the unit dosage can be packaged compositions, e.g., packeted powders, vials, ampoules, prefilled syringes or sachets containing liquids.

Suitably, the kit contains packaging or a container with the one or more compound of formula (I), (IIA), (IIB), (IIIC), (IV), and (V) formulated for the desired use or delivery route. Suitably, the kit contains instructions on use and an insert regarding the one or more compound of formula (I), (IIA), (IIB), (IIIC), (IV), and (V). Optionally, the kit may further contain instructions for monitoring efficacy of the compound and materials for performing such assays including, e.g., reagents, well plates, containers, markers or labels, and the like. Such kits are readily packaged in a manner suitable for treatment of a desired indication. For example, the kit may also contain instructions. Other suitable components to include in such kits will be readily apparent to one of skill in the art.

The one or more compound of formula (I), (IIA), (IIB), (IIIC), (IV), and (V) or composition of these kits also may be provided in dried or lyophilized forms. When reagents or components are provided as a dried form, reconstitution generally is by the addition of a suitable solvent. It is envisioned that the solvent also may be provided in another packaging means.

The kits may include a means for containing the vials in close confinement for commercial sale such as, e.g., injection or blow-molded plastic containers into which the desired vials are retained.

Irrespective of the number or type of packages, the kits also may include, or be packaged with a separate instrument for assisting with the injection/administration or placement of the ultimate complex composition within the body of an animal. Such an instrument may be an inhalant, syringe, pipette, forceps, measuring spoon, eye dropper or any such medically approved delivery means.

In some embodiments, pharmaceuticals kit are provided and contain one or more compound of formula (I), (IIA), (IIB), (IIIC), (IV), and (V). The one or more compound of formula (I), (IIA), (IIB), (IIIC), (IV), and (V) may be in the presence or absence of one or more of the carriers or excipients described above. The kit may optionally contain an additional active agent as described above and/or instructions for administering the additional active agent and the one or more compound of formula (I), (IIA), (IIB), (IIIC), (IV), and (V) to a subject.

In further embodiments, pharmaceutical kits are provided and contain an additional active agent in a first dosage unit, one or more compound of formula (I), (IIA), (IIB), (IIIC), (IV), and (V) as described herein in a second dosage unit, and one or more of the carriers or excipients in a third dosage unit. The kit may optionally contain instructions for administering the additional active agent and/or one or more compound of formula (I), (IIA), (IIB), (IIIC), (IV), and (V) to a subject.

Further provided are products containing one or more compound discussed herein. Thus, the products containing the compound of formula (I) has anti-bacterial or bacteriostatic activity. In some embodiments, the product is a dental care product that contains one or more compounds of Formula (I). The dental care product may be a mouthwash, toothpaste, toothbrush sanitizer, teeth whitener, floss, floss pick, fluoride treatment solution, or denture cream, among others. In some embodiments, the dental care product is a mouthwash or a toothpaste.

The compounds of formula (I) may also be incorporated into a cleaning product. In some embodiments, the cleaning product is for hygienic use. Thus, the compounds described herein may be incorporated into bath soaps including liquid or solid soaps; hair products such as shampoos, conditioners, styling agents, or coloring agents; lotions such as hand or body lotions; hand-sanitizers; perfumes; deodorants; or make-up, among others. In other embodiments, the cleaning product is for non-hygienic use. Thus, the compounds described herein may be incorporated into household cleaning products.

The compounds of formula (I) may further be incorporated into substrates. In some embodiments, the present disclosure provides plastics which contain the compound of formula (I). In other embodiments, the present disclosure provides composites which contain the compound of formula (I). In further embodiments, the present disclosure provides prostheses for dental care which are formed from plastics or composites containing the compound of formula (I). The prosthesis may be selected as determined by the patient, condition, among others. In some embodiments, the prosthesis is a denture, partial denture, palatal obturator, orthodontic appliance, dental implant, crown, bridge, or combination thereof.

The following Examples are provided to illustrate some of the concepts described within this disclosure. While each Example is considered to provide specific individual embodiments of composition, methods of preparation and use, none of the Examples should be considered to limit the more general embodiments described herein.

In the following examples, efforts have been made to ensure accuracy with respect to numbers used (e.g. amounts, temperature, etc.) but some experimental error and deviation should be accounted for. Unless indicated otherwise, temperature is in degrees C., pressure is at or near atmospheric.

### V. ASPECTS

Aspect 1: A compound of formula (I):

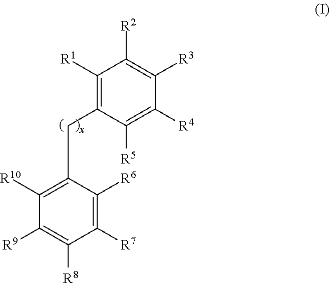

wherein:
R¹ and R⁵ are, independently, H, OH, C₁₋₆alkyl, or C₃₋₈cycloalkyl;
R² and R⁴ are, independently, H, OH, C₁₋₆alkyl, C₂₋₆alkenyl, C₁₋₆alkoxy, or C₃₋₈cycloalkyl;
R³ and R⁸ are, independently, H, OH, C₁₋₆alkyl, C₃₋₈cycloalkyl, and optionally substituted aryl;
R⁶ and R¹⁰ are, independently, H, OH, C₁₋₆alkyl, or C₃₋₈cycloalkyl;
R⁷ and R⁹ are, independently, H, OH, C₁₋₆alkyl, C₂₋₆alkenyl, C₃₋₈cycloalkyl, optionally substituted aryl;
or R⁹ and R¹⁰ are joined to form —C(R^{9′})═C(R¹¹)—C(R¹²)═C(R^{10′})—;
R^{9′}, R^{10′}, R¹¹ and R¹² are, independently, H, OH, halogen, C₁₋₆alkyl, C₁₋₆alkoxy, C₃₋₈cycloalkyl, C(O)(C₁₋₆alkyl), optionally substituted aryl, or optionally substituted heteroaryl; and
x is 0 to 8;
with the proviso that (i) at least one of R¹ to R⁵ is OH; (ii) at least one of R⁶ to R¹⁰ is OH; and (iii) the compound is not:
[1,1′-biphenyl]-2,2′-diol,
[1,1′-biphenyl]-2,4′-diol,
[1,1′-biphenyl]-3,3′-diol,
[1,1′-biphenyl]-4,4′-diol,
3,3′,6,6′-tetramethyl-[1,1′-biphenyl]-2,2′-diol,
2,2′,3,3′,6,6′-hexamethyl-[1,1′-biphenyl]-4,4′-diol;
3,3′-diallyl-4,4′,6,6′-tetramethyl-[1,1′-biphenyl]-2,2′-diol,
4,4′,6,6′-tetramethyl-3,3′-dipropyl-[1,1′-biphenyl]-2,2′-diol,
4,4′-dimethoxy-3,3′,6,6′-tetramethyl-[1,1′-biphenyl]-2,2′-diol,
[1,1′-biphenyl]-3,3′,4-triol,
[1,1′-biphenyl]-3,3′,4′,5′-tetraol,
3,3′,6,6′-tetramethyl-[1,1′-biphenyl]-2,2′,4,4′-tetraol,
4′,5″-dimethyl-[1,1′:2′,1″:2″,1′″-quaterphenyl]-3′,4″,5′,6″-tetraol, or
3,3′-dimethyl-6,6′-di(oct-1-yn-1-yl)-[1,1′-biphenyl]-2,2′,4,4′-tetraol,
   or a pharmaceutically acceptable salt thereof.

Aspect 2: The compound of aspect 1, wherein R¹ and/or R⁵ is H.

Aspect 3: The compound of aspect 1, wherein R¹ and/or R⁵ is OH, preferably R⁵ is OH.

Aspect 4: The compound of aspect 1, wherein R¹ and/or R⁵ is C₁₋₆alkyl, such as methyl.

Aspect 5: The compound of aspect 1, wherein R² and/or R⁴ is H.

Aspect 6: The compound of any one of the preceding aspects, wherein R² and/or R⁴ is C₁₋₆alkyl, such methyl, ethyl, propyl, butyl, pentyl, or hexyl, or preferably methyl, n-propyl, propyl, or t-butyl.

Aspect 7: The compound of any one of aspects 1 to 4, wherein R² and/or R⁴ is C₂₋₆alkenyl, such as ethenyl, propenyl, butenyl, pentenyl, or hexenyl, or preferably allyl.

Aspect 8: The compound of any one of aspects 1 to 4, wherein R² and/or R⁴ is C₁₋₆alkoxy, such as methoxy, ethoxy, propoxy, butoxy, pentoxy, or hexoxy, or preferably methoxy.

Aspect 9: The compound of any one of aspects 1 to 4, wherein R² and/or R⁴ is OH, preferably is R² is OH.

Aspect 10: The compound of any one of the preceding aspects, wherein R³ and/or R⁸ is C₁₋₆alkyl, such as methyl, ethyl, propyl, butyl, pentyl, or hexyl, or preferably methyl or t-butyl.

Aspect 11: The compound of any one of aspects 1 to 9, wherein R³ and/or R⁸ is OH.

Aspect 12: The compound of any one of aspects 1 to 9, wherein R³ and/or R⁸ is H.

Aspect 13: The compound of any one of aspects 1 to 9, wherein R³ and/or R⁸ is optionally substituted aryl, such as optionally substituted phenyl.

Aspect 14: The compound of any one of the preceding aspects, wherein R⁶ and/or R¹⁰ is H.

Aspect 15: The compound of any one of aspects 1 to 13, wherein R⁶ and/or R¹⁰ is OH, preferably R⁶ is OH.

Aspect 16: The compound of any one of aspects 1 to 13, wherein R⁶ and/or R¹⁰ is C₁₋₆alkyl, such as methyl, ethyl, propyl, butyl, pentyl, or hexyl, or preferably methyl or i-propyl.

Aspect 17: The compound of any one of the preceding aspects, wherein R⁷ and/or R⁹ is H.

Aspect 18: The compound of any one of aspects 1 to 16, wherein R⁷ and/or R⁹ is OH, preferably R⁷ is OH.

Aspect 19: The compound of any one of aspects 1 to 16, wherein R⁷ and/or R⁹ is C₁₋₆alkyl, such as methyl, ethyl, propyl, butyl, pentyl, or hexyl, or preferably methyl or n-propyl.

Aspect 20: The compound of any one of aspects 1 to 16, wherein R⁷ and/or R⁹ is C₂₋₆alkenyl, such as ethenyl, propenyl, butenyl, pentenyl, or hexenyl, or preferably propenyl.

Aspect 21: The compound of any one of aspects 1 to 16, wherein R⁷ and/or R⁹ are optionally substituted aryl, such as optionally substituted phenyl.

Aspect 22: The compound of any one of aspects 1 to 13, wherein R⁹ and R¹⁰ are joined to form —C(R^{9′})═C(R¹¹)—C(R¹²)═C(R^{10′})—.

Aspect 23: The compound of aspect 1 or 22, wherein R^{9′} and/or R^{10′} is H.

Aspect 24: The compound of aspect 1, 22, or 23, wherein R¹¹ and/or R¹² is H.

Aspect 25: The compound of aspect 1 or 22, wherein one or more of R^{9′}, R^{10′}, R¹¹, or R¹² is OH, preferably R¹² is OH.

Aspect 26: The compound of aspect 1 or 22, wherein one or more of R^{9′}, R^{10′}, R¹¹, or R¹² is halogen, preferably bromo.

Aspect 27: The compound of aspect 1 or 22, wherein one or more of R^{9′}, R^{10′}, R¹¹, or R¹² is C₁₋₆alkoxy, preferably methoxy.

Aspect 28: The compound of aspect 1 or 22, wherein one or more of R^{9′}, R^{10′}, R¹¹, or R¹² is heteroaryl, such as pyrazolyl.

Aspect 29: The compound of aspect 1 or 22, wherein one or more of R^{9′}, R^{10′}, R¹¹, or R¹² is C₁₋₆alkenyl, such as t-butyl.

Aspect 30: The compound of aspect 1 or 22, wherein one or more of R^{9′}, R^{10′}, R¹¹, or R¹² is C(O)C₁₋₆alkoxy, such as C(O)OCH₃.

Aspect 31: The compound of any one of the preceding aspects, wherein x is 2.

Aspect 32: The compound of aspect 1 of formula (IIA):

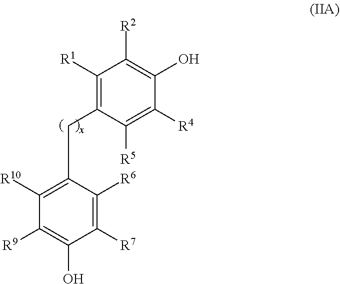

or a salt thereof.

Aspect 33: The compound of aspect 32, wherein x is 0.

Aspect 34: The compound of aspect 1 of formula (IIB):

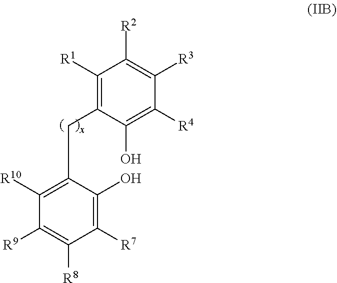

or a salt thereof.

Aspect 35: The compound of aspect 1 of formula (IIC):

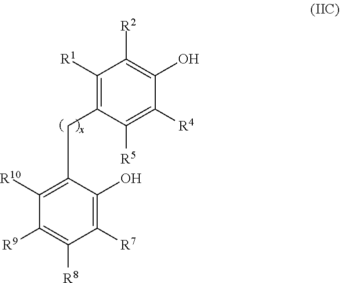

or a salt thereof.

Aspect 36: The compound of aspect 1 of formula (III):

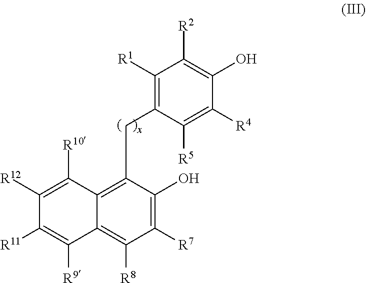

or a salt thereof.

Aspect 37: The compound of aspect 36, wherein x is 0.

Aspect 38: The compound of aspect 36 or 37, wherein R² and R⁴ are C₁₋₆alkyl.

Aspect 39: The compound of aspect 36 or 37, wherein R² is C₁₋₆alkyl and R⁴ is C₂₋₆alkenyl.

Aspect 40: The compound of aspect 36 or 37, wherein R² and R⁴ are C₁₋₆alkoxy.

Aspect 41: The compound of any one of aspects 36 to 40, wherein R¹ and R⁵ are H and/or R⁷ and R⁸ are H and/or R^{9′}, R^{10′}, R¹¹, and R¹² are H.

Aspect 42: The compound of aspect 1, that is:

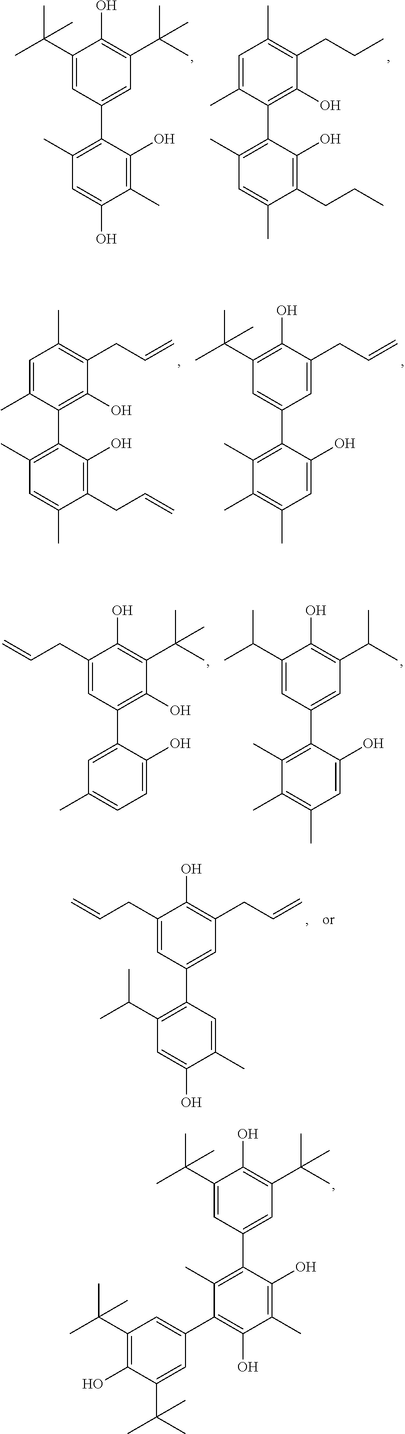

or a salt thereof.

Aspect 43: The compound of aspect 1, that is:

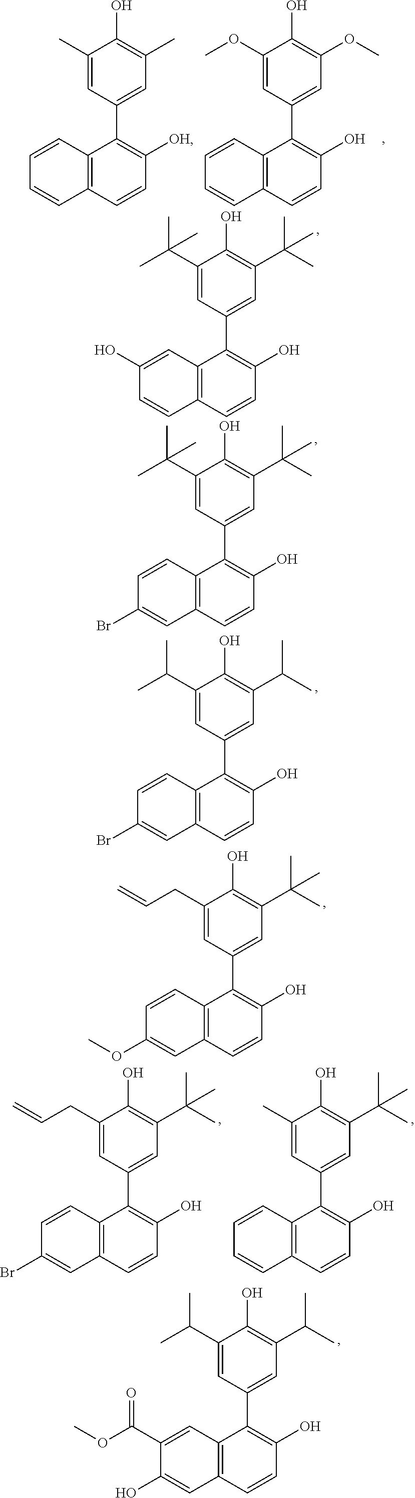

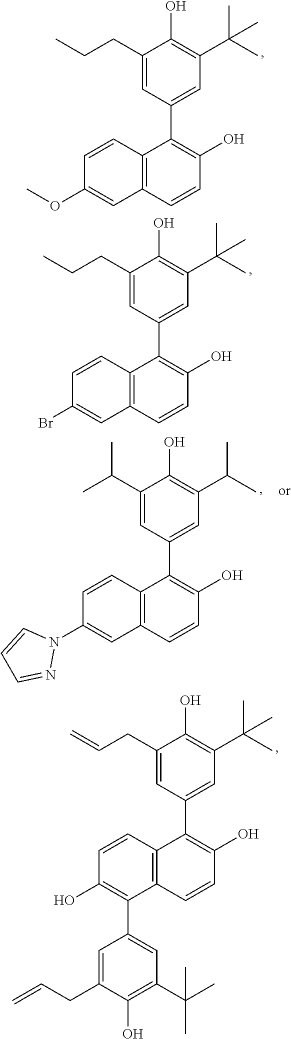

or a salt thereof.

Aspect 44: The compound of aspect 1, that is:

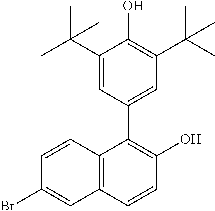 [SMILES: CC(C)(C)c1cc(-c2c(O)ccc3cc(Br)ccc23)cc(C(C)(C)C)c1O]

or a salt thereof.

Aspect 45: The compound of aspect 1, that is:

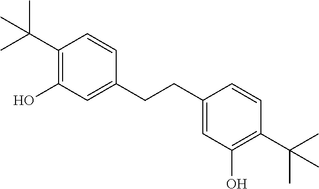 [SMILES: CC(C)(C)c1ccc(CCc2ccc(C(C)(C)C)c(O)c2)cc1O]

or a salt thereof.

Aspect 46: A composition comprising one or more compounds of any one of the preceding aspects and an excipient.

Aspect 47: The composition of aspect 46, wherein the excipient is a topical pharmaceutical excipient.

Aspect 48: The composition of aspect 46, wherein the excipient is a dental care additive such as a toothpaste additive or mouth rinse.

Aspect 49: The composition of aspect 46, wherein the excipient is a soap.

Aspect 50: The composition of aspect 46, wherein the excipient is a sterilization agent.

Aspect 51: The composition of aspect 46, wherein the excipient is a plastic.

Aspect 52: A method of inhibiting, reducing, or ameliorating bacterial growth on a substrate, comprising contacting the substrate with a compound of formula (I):

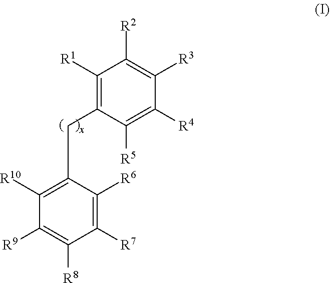

wherein:

R¹ and R⁵ are, independently, H, OH, C₁₋₆alkyl, or C₃₋₈cycloalkyl;

R² and R⁴ are, independently, H, OH, C₁₋₆alkyl, C₂₋₆alkenyl, C₁₋₆alkoxy, or C₃₋₈cycloalkyl;

R³ and R⁸ are, independently, H, OH, C₁₋₆alkyl, C₃₋₈cycloalkyl, and optionally substituted aryl;

R⁶ and R¹⁰ are, independently, H, OH, C₁₋₆alkyl, or C₃₋₈cycloalkyl;

R⁷ and R⁹ are, independently, H, OH, C₁₋₆alkyl, C₂₋₆alkenyl, C₃₋₈cycloalkyl, optionally substituted aryl;

or R⁹ and R¹⁰ are joined to form) —C(R^{9′})═C(R¹¹)—C(R¹²)═C(R^{10′})—;

R^{9′}, R^{10′}, R¹¹ and R¹² are, independently, H, OH, halogen, C₁₋₆alkyl, C₁₋₆alkoxy, C₃₋₈cycloalkyl, C(O)(C₁₋₆alkyl), optionally substituted aryl, or optionally substituted heteroaryl; and

x is 0 to 8;

with the proviso that (i) at least one of R¹ to R⁵ is OH; (ii) at least one of R⁶ to R¹⁰ is OH; and (iii) the compound is not 3′,5-diallyl-[1,1′-biphenyl]-2,4′-diol, or a pharmaceutically acceptable salt thereof.

Aspect 53: The method of aspect 52, wherein the bacterial growth is a biofilm.

Aspect 54: The method of aspect 52 or 53, wherein the substrate is skin or an oral cavity.

Aspect 55: The method of aspect 52 or 53, wherein the substrate is the heart, lung, or combination thereof.

Aspect 56: The method of any one of aspects 52 to 55, wherein the bacterium is a pathogenic bacterium, such as an oral pathogenic bacterium.

Aspect 57: The method of any one of aspects 52 to 56, wherein the bacterium is gram positive or gram negative bacteria, such as one or more of S. mutans, S. sanguinis, or S. gordonii.

Aspect 58: The method of any one of aspects 52 to 56, wherein the bacterium is a Streptococcal strain.

Aspect 59: A dental care product, comprising one or more compounds of any one of aspects 1 to 45.

Aspect 60: A soap, comprising one or more compounds of any one of aspects 1 to 45.

Aspect 61: An antibacterial product, comprising one or more compounds of any one of aspects 1 to 45.

### VI. EXAMPLES

Compounds for which no synthetic procedure is specifically recited may be purchased commercially. For example, bis(2-hydroxyphenyl)methane and 2-(tert-butyl)-5-methylphenolmay be purchased from Sigma Aldrich. Compounds A3, A7, and B3 in Table 1 were prepared according to the procedure described in Lee, J. Am. Chem. Soc. 2014, 136, 6782-6785 in yields of 63%, 74%, and 83%, respectively.

TABLE 1

| | |
|---|---|
| 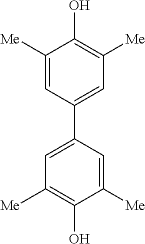 [SMILES: Cc1cc(-c2cc(C)c(O)c(C)c2)cc(C)c1O] | A3 |
| 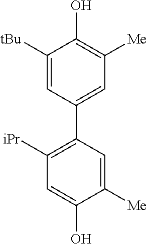 [SMILES: Cc1cc(-c2cc(C)c(O)c(C(C)(C)C)c2)c(C(C)C)cc1O] | A7 |
| 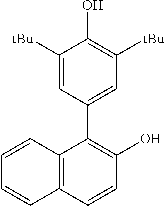 [SMILES: CC(C)(C)c1cc(-c2c(O)ccc3ccccc23)cc(C(C)(C)C)c1O] | B3 |

### Example 1: General Procedure A for Oxidative Coupling of Phenols (x=0)

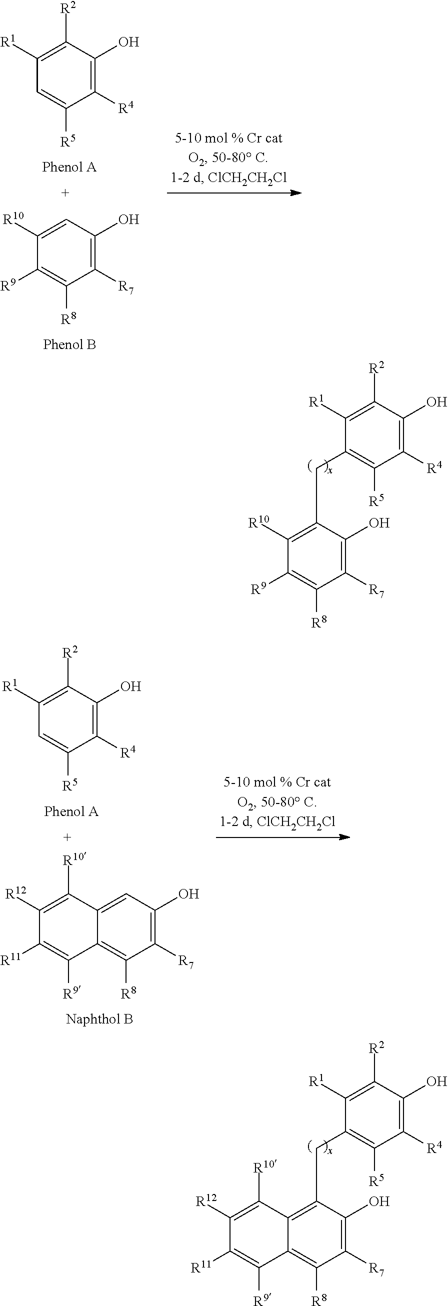

TABLE 2

| | Phenol A | | | Phenol B | | | | Naphthol B | |
|---|---|---|---|---|---|---|---|---|---|
| Product | R² | R⁴ | R⁵ | R⁷ | R⁸ | R⁹ | R¹⁰ | R¹¹ | R¹² |
| A6 | allyl | t-Bu | OH | H | Me | Me | Me | — | — |
| A8 | allyl | t-Bu | OH | H | H | Me | H | — | — |
| A9 | i-Pr | i-Pr | H | H | Me | Me | Me | — | — |
| A10 | allyl | allyl | H | Me | H | H | i-Pr | — | — |
| B5 | t-Bu | t-Bu | H | — | — | — | — | H | Br |
| B6 | i-Pr | i-Pr | H | — | — | — | — | H | Br |
| B7 | allyl | t-Bu | H | — | — | — | — | H | OMe |
| B8 | allyl | t-Bu | H | — | — | — | — | H | Br |
| B9 | Me | t-Bu | H | — | — | — | — | H | H |
| B10 | i-Pr | i-Pr | H | — | — | — | — | CO₂Me | OH |
| B11 | n-Pr | t-Bu | H | — | — | — | — | H | OMe |
| B12 | n-Pr | t-Bu | H | — | — | — | — | H | Br |
| B13 | i-Pr | i-Pr | H | — | — | — | — | H | pyrazolyl |
| B14 | allyl | t-Bu | H | — | — | — | — | H | OH |

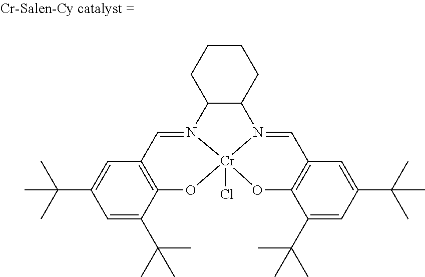

To a 10 mL microwave vial was added phenol A (0.1 mmol), phenol B (0.15 mmol), and Cr-Salen-Cy catalyst (0.01 mmol). The vial was sealed with a septum and ClCH₂CH₂Cl (2.5 mL) was added. Oxygen was added via active purge. The septum was replaced with a crimping cap and the vessel was sealed and stirred for the indicated time at 80° C. After the reaction mixture was filtered through a plug of silica and concentrated, the material was chromatographed using 10-20% ethyl acetate/hexane to afford the product.

### Example 2: 6-Bromo-1-(3,5-di-tert-butyl-4-hydroxyphenyl)naphthalen-2-ol (B5)

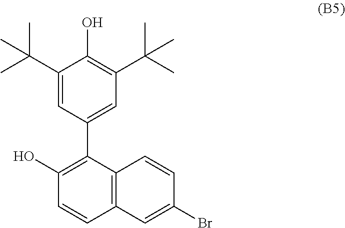 [SMILES: CC(C)(C)c1cc(-c2c(O)ccc3cc(Br)ccc23)cc(C(C)(C)C)c1O]

Following general procedure A for 2 d, the cross-coupled product was obtained as a red-orange solid (0.0103 g, 0.0241 mmol) in a 24% yield: ¹H NMR (500 MHz, CDCl₃) δ 7.95 (d, J=2.0 Hz, 1H), 7.68 (d, J=9.0 Hz, 1H), 7.41 (dd, J=9.0 Hz, 2.0 Hz, 1H), 7.34 (d, J=9.0 Hz, 1H), 7.28 (d, J=8.5 Hz, 1H), 7.16 (s, 2H), 5.41 (s, 1H), 5.35 (s, 1H), 1.47 (s, 18H); ¹³C NMR (125 MHz, CDCl₃) δ 154.3, 150.9, 137.5, 132.4, 130.2, 130.0, 129.7, 128.2, 127.6, 127.0, 124.0, 122.3, 118.4, 117.0, 34.7, 30.5; IR (neat) 3632, 3527, 2958, 1615, 1588, 1502, 1437, 1403, 1377, 1362, 1340, 1309, 1236, 1174, 1148, 1120, 958, 885, 821, 511 cm¹; HRMS (ESI-TOF) m/z=425.1116 calc for C₂₄H₂₆BrO₂ [M−H]⁻, found 425.1141.

### Example 3: 6-Bromo-1-(4-hydroxy-3,5-diisopropylphenyl)naphthalen-2-ol (B6)

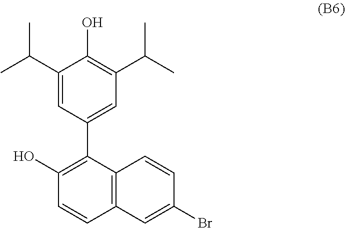 [SMILES: CC(C)c1cc(-c2c(O)ccc3cc(Br)ccc23)cc(C(C)C)c1O]

Following general procedure A for 2 d, the cross-coupled product was obtained as a light orange solid (0.0314 g, 0.0786 mmol) in a 72% yield: ¹H NMR (500 MHz, CDCl₃) δ 7.95 (d, J=2.0 Hz, 1H), 7.69 (d, J=9.0 Hz, 1H), 7.40 (dd, J=9.0 Hz, 2.0 Hz, 1H) 7.32 (d, J=9.0 Hz, 1H), 7.28 (d, J=9.0 Hz, 1H), 7.06 (s, 2H), 5.32 (s, 1H), 5.00 (s, 1H), 3.24 (sept, J=6.9, 2H), 1.30 (d, J=6.8 Hz, 6H), 1.29 (d, J=6.9 Hz, 6H); ¹³C NMR (125 MHz, CDCl₃) δ 150.8, 150.5, 135.3, 132.4, 130.2, 130.0, 129.7, 128.2, 126.9, 126.2, 125.1, 122.1, 118.4, 117.1, 27.5, 22.9, 22.8; IR (neat) 3521, 2961, 2927, 1588, 1501, 1461, 1445, 1376, 1362, 1340, 1293, 1258, 1195, 1169, 1148, 1136, 1119, 1071, 882, 825, 812, 470, 452 cm⁻¹; HRMS (ESI-TOF) m/z=397.0803 calc for C₂₂H₂₂BrO₂ [M+H]⁺, found 397.0809.

### Example 4: 3′,5′-Diisopropyl-4,5,6-trimethyl-[1,1′-biphenyl]-2,4′-diol (A9)

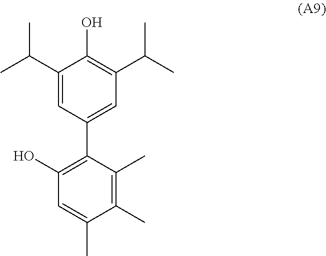 [SMILES: Cc1cc(O)c(-c2cc(C(C)C)c(O)c(C(C)C)c2)c(C)c1C]

Following general procedure A for 2 d, the cross-coupled product was obtained as a white solid (0.0056 g, 0.0179 mmol) in an 18% yield: ¹H NMR (500 MHz, CDCl₃) δ 6.93 (s, 2H), 6.72 (s, 1H), 4.90 (s, 1H), 4.73 (s, 1H), 3.19 (sept, J=7.0 Hz, 2H), 2.30 (s, 3H), 2.16 (s, 3H), 2.00 (s, 3H), 1.27-1.25 (m, 12H); ¹³C NMR (125 MHz, CDCl₃) δ 150.5, 149.7, 136.7, 135.5, 134.7, 127.7, 126.8, 126.6, 125.7, 113.6, 27.2, 22.8, 22.7, 20.8, 17.8, 15.4; IR (neat) 3426, 2960, 2927, 2870, 1589, 1465, 1383, 1363, 1292, 1251, 1199, 1173, 1150, 1112, 1042, 938, 882, 786, 726, 660 cm¹; HRMS (ESI-TOF) m/z=313.2168 calc for C₂₁H₂₉O₂ [M+H]⁺, found 313.2191.

### Example 5: 1-(3-Allyl-5-(tert-butyl)-4-hydroxyphenyl)-6-bromonaphthalen-2-ol (B8)

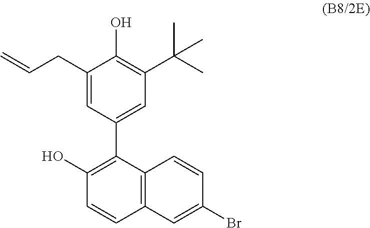 [SMILES: C=CCc1cc(-c2c(O)ccc3cc(Br)ccc23)cc(C(C)(C)C)c1O]

Following general procedure A for 2 d, the cross-coupled product was obtained as a red-orange solid (0.0681 g, 0.1656 mmol) in an 85% yield: ¹H NMR (500 MHz, CDCl₃) δ 7.95 (d, J=2.0 Hz, 1H), 7.68 (d, J=9.0 Hz, 1H), 7.40 (dd, J=7.0 Hz, 2.0 Hz, 1H), 7.32 (d, J=9.0 Hz, 1H), 7.27 (d, J=9.0 Hz, 1H), 7.18 (d, J=2.0 Hz, 1H), 7.01 (d, J=2.0 Hz, 1H), 6.07 (m, 1H), 5.47 (s, 1H), 5.31 (m, 3H), 3.49 (d, J=6.0 Hz, 2H), 1.43 (s, 9H); ¹³C NMR (125 MHz, CDCl₃) δ 154.1, 150.8, 138.5, 136.0, 132.4, 130.7, 130.2, 130.0, 129.7, 128.5, 128.3, 126.9, 126.5, 124.7, 121.7, 118.4, 118.0, 117.1, 36.4, 35.1, 29.9; IR (neat) 3517, 2956, 1725, 1587, 1500, 1475, 1414, 1376, 1362, 1339, 1263, 1226, 1196, 1170, 1144, 1068, 1043, 957, 892, 878, 820, 737, 465, 455 cm¹; HRMS (ESI-TOF) m/z=411.0960 calc for C₂₃H₂₄BrO₂ [M+H]⁺, found 411.0939.

### Example 6: 1-(3-Allyl-5-(tert-butyl)-4-hydroxyphenyl)-6-methoxynaphthalen-2-ol (B7)

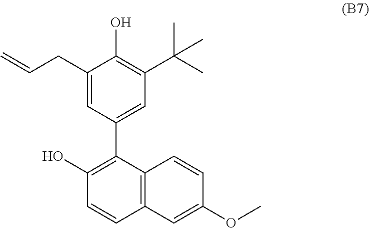 [SMILES: C=CCc1cc(-c2c(O)ccc3cc(OC)ccc23)cc(C(C)(C)C)c1O]

Following general procedure A for 2 d, the cross-coupled product was obtained as a dark yellow solid (0.0181 g, 0.0499 mmol) in a 49% yield: ¹H NMR (500 MHz, CDCl₃) δ 7.67 (d, J=9.0 Hz, 1H) 7.37 (d, J=9.0 Hz, 1H), 7.23 (d, J=9.0 Hz, 1H), 7.21 (d, J=2.0 Hz, 1H), 7.14 (d, J=2.5 Hz, 1H), 7.04 (dd, J=9.3 Hz, 2.3 Hz, 1H), 7.02 (s, 1H), 6.07 (m, 1H), 5.42 (s, 1H), 5.30 (m, 2H), 5.13 (s, 1H), 3.91 (s, 3H), 3.49 (d, J=6.5 Hz, 2H), 1.43 (s, 9H); ¹³C NMR (125 MHz, CDCl₃) δ 155.9, 153.9, 148.9, 138.3, 136.1, 130.8, 129.9, 129.0, 128.6, 127.9, 126.5, 126.2, 125.5, 121.7, 119.0, 117.9, 117.7, 55.5, 36.4, 35.1, 29.9; IR (neat) 3530, 2956, 1600, 1515, 1464, 1426, 1374, 1350, 1235, 1170, 1139, 1115, 1073, 1040, 922, 851, 825, 650, 596 cm⁻¹, HRMS (ESI-TOF) m/z=363.1960 calc for C₂₄H₂₇O₃ [M+H]⁺, found 363.1974.

### Example 7: 1,5-Bis(3-allyl-5-(tert-butyl)-4-hydroxyphenyl)naphthalene-2,6-diol (B14)

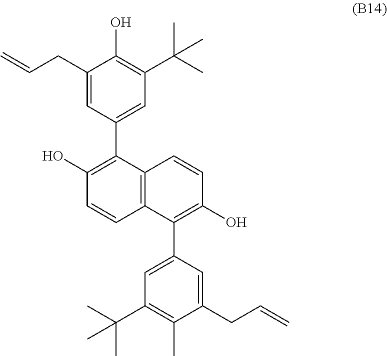 [SMILES: C=CCc1cc(-c2c(O)ccc3c(-c4cc(CC=C)c(O)c(C(C)(C)C)c4)c(O)ccc23)cc(C(C)(C)C)c1C]

Following general procedure A for 2 d, the cross-coupled product was obtained as a purple solid (0.0112 g, 0.0209 mmol) in a 21% yield: ¹H NMR (500 MHz, CDCl₃) δ 7.40 (d, J=9.1, 2H), 7.23 (s, 2H), 7.14 (d, J=9.1, 2H), 7.05 (s, 2H), 6.07 (m, 2H), 5.44 (s, 2H), 5.33 (m, 4H), 5.11 (s, 2H), 3.50 (d, J=6.4, 4H), 1.44 (s, 18H); ¹³C NMR (125 MHz, CDCl₃) δ 174.3, 153.9, 148.6, 138.3, 136.1, 130.8, 129.0, 128.7, 126.3, 126.1, 125.6, 121.8, 117.9, 117.5, 36.5, 35.1, 29.9; IR (neat) 3420, 3302, 2958, 1596, 1514, 1467, 1400, 1362, 1332, 1187, 1114, 1000, 917, 818, 677, 600 cm⁻¹; HRMS (ESI-TOF) m/z=537.3005 calc for C₃₆H₄₁O₄ [M+H]⁺, found 537.2991.

### Example 8: 3′-Allyl-5′-(tert-butyl)-5-methyl-[1,1′-biphenyl]2,4′-diol (A8)

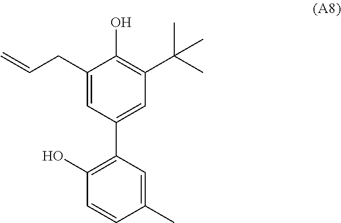 [SMILES: C=CCc1cc(-c2cc(C)ccc2O)cc(C(C)(C)C)c1O]

Following general procedure A, phenol A (0.5 mmol), phenol B (0.1 mmol), and Cr-Salen-Cy (0.01 mmol) in ClCH₂CH₂Cl (2.5 mL) were heated at 50° C. for 1 d, and the cross-coupled product was obtained as a red-orange amorphous solid (0.0109 g, 0.0368 mmol) in 28% yield: ¹H NMR (500 MHz, CDCl₃) δ 7.25 (d, J=2.8 Hz, 1H), 7.07 (d, J=2.2 Hz, 1H), 7.04-7.02 (m, 2H), 6.87 (d, J=7.9 Hz, 1H), 6.08-6.02 (m, 1H), 5.34 (s, 1H), 5.31-5.26 (m, 2H), 5.13 (s, 1H), 3.46 (d, J=6.0 Hz, 2H), 2.31 (s, 3H), 1.43 (s, 9H); ¹³C NMR (125 MHz, CDCl₃) δ 153.4, 150.3, 137.9, 135.9, 130.6, 129.8, 129.1, 128.7, 128.7, 128.1, 126.6, 125.9, 117.7, 115.3, 36.3, 34.9, 29.8, 20.5; IR (neat) 3533, 2954, 2919, 2867, 1497, 1471, 1449, 1392, 1361, 1323, 1253, 1230, 1209, 1175, 1125, 999, 919, 876, 815, 712 cm¹; HRMS (ESI) m/z=295.1698 calc for C₂₀H₂₃O₂ [M−H]⁻, found 295.1715.

### Example 9: 3′-Allyl-5′-(tert-butyl)-4,5,6-trimethyl-[1,1′-biphenyl]2,4′-diol (A6)

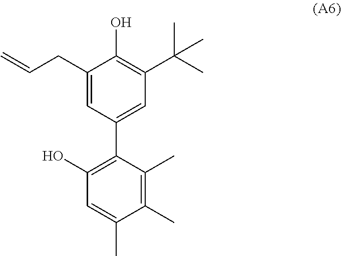 [SMILES: C=CCc1cc(-c2c(O)cc(C)c(C)c2C)cc(C(C)(C)C)c1O]

Following general procedure A, phenol A (0.5 mmol), phenol B (0.1 mmol), and Cr-Salen-Cy (0.01 mmol) in ClCH₂CH₂Cl (2.5 mL) were heated at 50° C. for 1 d, and the cross-coupled product was obtained as a yellow solid (0.0113 g, 0.0348 mmol) in a 33% yield: ¹H NMR (500 MHz, CDCl₃) δ 7.05 (d, J=2.0 Hz, 1H), 6.87 (d, J=2.0 Hz, 1H), 6.71 (s, 1H), 6.09-6.01 (m, 1H), 5.32 (s, 1H), 5.29-5.25 (m, 2H), 4.69 (s, 1H), 3.44 (d, J=6.5 Hz, 2H), 2.30 (s, 3H), 2.16 (s, 3H), 2.00 (s, 3H), 1.40 (s, 9H); ¹³C NMR (125 MHz, CDCl₃) δ 153.4, 150.7, 138.0, 136.9, 136.2, 135.7, 130.2, 128.2, 127.3, 127.0, 126.4, 126.0, 117.7, 113.8, 36.4, 35.0, 29.9, 21.0, 18.0, 15.5; IR (neat) 3532, 2956, 2925, 2870, 1600, 1443, 1391, 1362, 1201, 1087, 1037, 999, 919, 798, 748, 701 cm⁻¹; HRMS (EI-TOF) m/z=324.2089 calc for C₂₂H₂₈O₂ [M]⁺, found 324.2095.

### Example 10: Methyl 3,7-dihydroxy-8-(4-hydroxy-3,5-diisopropylphenyl)-2-naphthoate (B10)

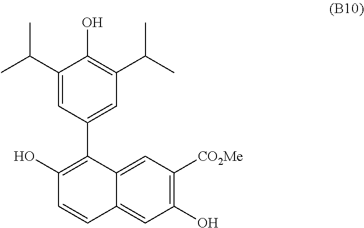 [SMILES: COC(=O)c1cc2c(-c3cc(C(C)C)c(O)c(C(C)C)c3)c(O)ccc2cc1O]

Following general procedure A at 55° C. for 2 d, the cross-coupled product was obtained as a yellow solid (0.022 g, 0.056 mmol) in a 56% yield: ¹H NMR (500 MHz, CDCl₃) δ 10.24 (s, 1H), 8.20 (s, 1H), 7.63 (d, J=9 Hz, 1H), 7.32 (d, J=9 Hz, 1H), 7.31 (s, 1H), 7.12 (s, 1H), 5.32 (s, 1H), 5.01 (s, 1H), 3.89 (s, 3H), 3.27 (septet, J=6.9 Hz, 2H), 1.32 (d, J=7.1 Hz, 6H) 1.28 (d, J=7.5, 6H); ¹³C NMR (125 MHz, CDCl₃) δ 170.4, 154.6, 150.2, 148.6, 135.2, 133.7, 129.2, 127.3, 127.0, 126.2, 125.1, 122.2, 121.2, 114.3, 112.1, 52.3, 27.3, 22.9, 22.7; IR (neat) 3521, 2961, 1682, 1523, 1472, 1442, 1415, 1386, 1346, 1323, 1294, 1243, 1198, 1171, 1150, 1122 cm⁻¹; HRMS (EI-TOF) m/z=394.1906 calc for C₂₄H₂₆O₅ [M]⁺, found 394.1905.

### Example 11: 1-(3-(tert-Butyl)-4-hydroxy-5-methylphenyl)naphthalen-2-ol (B9)

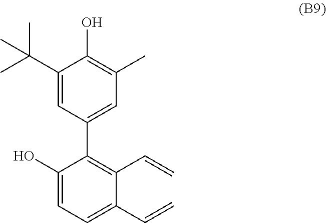 [SMILES: C=Cc1ccc(O)c(-c2cc(C)c(O)c(C(C)(C)C)c2)c1C=C]

Following general procedure A at 55° C. for 2 d, the cross-coupled product was obtained as a white solid (4.4 mg, 0.0144 mmol) in a 13% yield: ¹H NMR (500 MHz, CDCl₃) δ 7.79 (m, 2H), 7.46 (d, J=7.0 Hz, 1H), 7.33 (m, 2H), 7.26 (d, J=8.5 Hz, 1H), 7.17 (d, J=0.75 Hz, 1H), 7.06 (d, J=0.75 Hz, 1H), 5.27 (s, 1H), 4.96 (s, 1H), 2.34 (s, 3H), 1.45 (s, 9H); ¹³C NMR (125 MHz, CDCl₃) δ 152.9, 150.4, 137.1, 133.7, 131.0, 129.1, 128.9, 128.0, 127.9, 126.3, 125.0, 124.8, 124.3, 123.1, 121.4, 117.1, 34.8, 29.8, 16.1; IR (neat) 3523, 2956, 2924, 1620, 1597, 1481, 1465, 1435, 1389, 1362, 1345, 1313, 1263, 1223, 1190, and 1174 cm⁻¹; HRMS (EI-TOF) m/z=306.1620 calc for C₂₁H₂₂O₂ [M]⁺, found 306.1632.

### Example 12: 1-(4-Hydroxy-3,5-diisopropylphenyl)-6-(1H-pyrazol-1-yl)naphthalen-2-ol (B13)

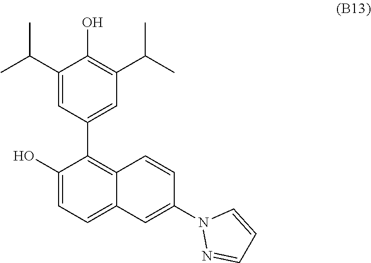 [SMILES: CC(C)c1cc(-c2c(O)ccc3cc(-n4cccn4)ccc23)cc(C(C)C)c1O]

Modifying general procedure A, 2 equiv of phenol A (0.0113 g, 0.0634 mmol) and 1.5 equiv of phenol B (0.010 g, 0.0476 mmol were treated at 55° C. for 2 d, and the cross-coupled product was obtained as a brown solid (6.9 mg, 0.0179 mmol) in a 28% yield: ¹H NMR (500 MHz, CDCl₃) δ 8.08 (s, 1H), 8.00 (s, 1H), 7.82 (d, J=9.0 Hz, 1H), 7.75 (d, J=0.8 Hz, 1H), 7.73 (dd, J=9.0, 1.2 Hz, 1H), 7.55 (d, J=9.0 Hz, 1H), 7.31 (d, J=9.0 Hz, 1H), 7.11 (s, 2H), 6.49 (t, J=2.0 Hz, 1H), 5.34 (s, 1H), 5.05 (s, 1H), 3.26 (septet, J=7.0, 2H), 1.31 (d, J=5.5 Hz, 6H), 1.30 (d, J=6.0 Hz, 6H); ¹³C NMR (125 MHz, CDCl₃) δ 150.6, 150.5, 141.2, 135.9, 135.3, 132.3, 129.1, 129.1, 127.1, 126.6, 126.3, 125.3, 122.0, 119.2, 118.5, 117.0, 107.7, 27.5, 23.0, 22.9; IR (neat) 3244, 2962, 2928, 2871, 1603, 1519, 1471, 1397, 1352, 1263, 1198, 1168, 1144, 1043, 954, 918 cm⁻¹; HRMS (ESI-TOF) m/z=387.2073 calc for C₂₅H₂₆N₂O₂ [M]⁺, found 387.2086.

### Example 13: 3′,5′-Diallyl-2-isopropyl-5-methyl-[1,1′-biphenyl]-4,4′-diol (A10)

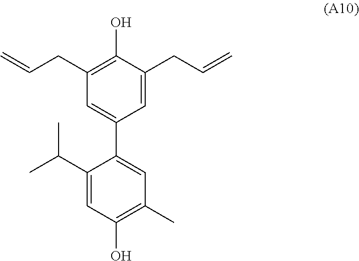 [SMILES: C=CCc1cc(-c2cc(C)c(O)cc2C(C)C)cc(CC=C)c1O]

Following general procedure A at 55° C. for 2 d, the cross-coupled product was obtained as a yellow oil (1.1 mg, 0.0034 mmol) in a 4% yield: ¹H NMR (500 MHz, CDCl₃) δ 6.93 (s, 1H), 6.90 (s, 2H), 6.76 (s, 1H), 6.04 (m, 2H), 5.12-5.2 (m 5H), 4.57 (s, 1H), 3.43 (d, J=7.5 Hz, 4H), 3.00 (septet, J=7.0 Hz, 1H), 2.22 (s, 3H), 1.12 (d, J=7.0 Hz, 6H); IR (neat) 3376, 2960, 1780, 1659, 1600, 1514, 1464, 1374, 1234, 1166, 1038, 909, 852, 824, 732, 704 cm⁻¹.

### Example 14: 6-Bromo-1-(3-(tert-butyl)-4-hydroxy-5-propylphenyl)naphthalen-2-ol (B12)

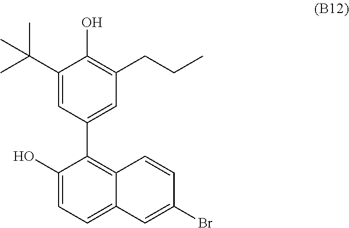 [SMILES: CCCc1cc(-c2c(O)ccc3cc(Br)ccc23)cc(C(C)(C)C)c1O]

To a solution of 1-(3-allyl-5-(tert-butyl)-4-hydroxyphenyl)-6-bromonaphthalen-2-ol (0.010 g, 0.024 mmol) in ethanol (1 mL, 0.02 M) at room temperature was added hydrazine monohydrate (0.010 g, 0.194 mmol). The mixture heated to reflux and stirred for 22 h. After concentration, the resulting solid was treated with H₂O and extracted using ethyl acetate (5×10 mL). The combined organic fractions were dried over Na₂SO₄, concentrated, and chromatographed (hexane/EtOAc, 10:1). The reduced product was obtained as a tan solid (9.3 mg, 0.0225 mmol) in a 93% yield: ¹H NMR (500 MHz, CDCl₃) δ 7.95 (d, J=2.0 Hz, 1H), 7.68 (d, J=9.0 Hz, 1H), 7.40 (dd, J=9.0, 1 Hz, 1H), 7.33 (d, J=9.0 Hz, 1H), 7.27 (d, J=8.5 Hz, 1H), 7.13 (d, J=2.0 Hz, 1H), 7.01 (d, J=2.0 Hz, 1H), 5.32 (broad, 1H), 5.08 (broad, 1H), 2.62 (dd, J=6.5, 1.5 Hz, 2H), 1.71 (m, 2H), 1.45 (s, 9H), 1.03 (t, J=7.5 Hz, 3H); ¹³C NMR (125 MHz, CDCl₃) δ 152.6, 150.6, 137.3, 132.2, 130.0, 129.9, 129.8, 129.4, 128.9, 128.0, 127.5, 126.7, 124.2, 121.7, 118.2, 116.8, 34.7, 32.0, 29.6, 22.6, 14.0; IR (neat) 3520, 2957, 2925, 1588, 1446, 1377, 1362, 1339, 1316, 1262, 1223, 1186, 1168, 1144, 1122, 1068, 1016 cm¹; HRMS (EI-TOF) m/z=412.1038 calc for C₂₃H₂₅BrO₂ [M]⁺, found 412.1059.

### Example 15: 1-(3-(tert-Butyl)-4-hydroxy-5-propylphenyl)-6-methoxynaphthalen-2-ol (B11)

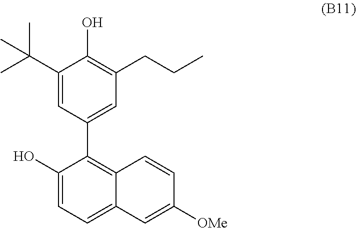 [SMILES: CCCc1cc(-c2c(O)ccc3cc(OC)ccc23)cc(C(C)(C)C)c1O]

To a stirring solution of allylated phenol (0.012 g, 0.033 mmol) in dry methanol (0.8 mL) was added Pd/C (0.010 mg). The reaction flask was evacuated and backfilled with H₂ (3 times). The reaction was stirred under H₂ atmosphere for 36 hours. The reaction mixture was then filtered through Celite and concentrated. Chromatography (hexane/EtOAc, 10:1) provided the reduced product was obtained as a tan solid (0.0021 g, 0.0058 mmol) in a 18% yield (˜15% impure): ¹H NMR (500 MHz, CDCl₃) δ 7.67 (d, J=8.5 Hz, 1H), 7.37 (d, J=9.0 Hz, 1H), 7.23 (d, J=9.0 Hz, 1H), 7.16 (d, J=2.0 Hz, 1H), 7.13 (d, J=2.0 Hz, 1H) 7.03 (m, 2H), 5.14 (broad, 1H), 5.04 (s, 1H), 3.91 (s, 3H), 2.62 (dd, J=7.0, 1.5 Hz, 2H), 1.72 (m, 2H), 1.45 (s, 9H), 1.03 (t, J=7.5 Hz, 3H); IR (neat) 3522, 2959, 1601, 1516, 1465, 1374 cm⁻¹ HRMS (ESI-TOF) m/z=363.1960 calc for C₂₄H₂₈O₃ [M−H]⁻, found 363.1944.

### Example 16: General Procedure B for Oxidative Coupling of Phenols (x=0)

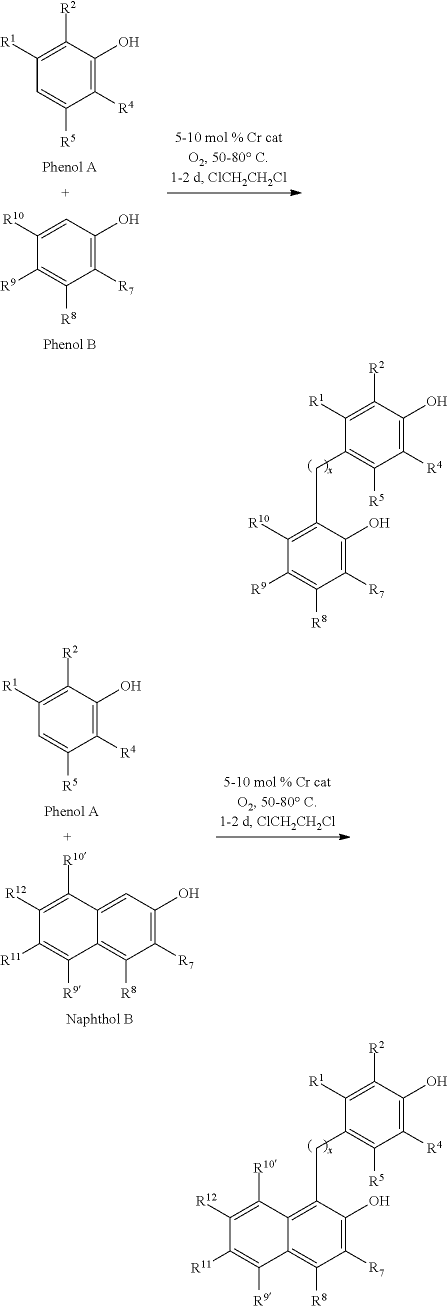

TABLE 3

| | Phenol A | | | Phenol B | | | | Naphthol B | |
|---|---|---|---|---|---|---|---|---|---|
| Product | R² | R⁴ | R⁵ | R⁷ | R⁸ | R⁹ | R¹⁰ | R¹¹ | R¹² |
| A2 | t-Bu | t-Bu | H | Me | OH | H | Me | — | — |
| A11 | t-Bu | t-Bu | H | Me | OH | H | Me | — | — |
| B1 | Me | Me | H | — | — | — | — | H | H |
| B2 | MeO | MeO | H | — | — | — | — | H | H |
| B4 | t-Bu | t-Bu | H | — | — | — | — | OH | H |

* any “R” group not specifically recited in the table may be H.

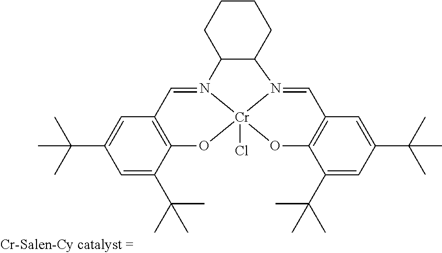

To a 5-mL microwave vial was added phenol A (0.3 mmol), phenol B (0.25 mmol) and Cr-Salen-Cy catalyst (0.025 mmol). The vial was sealed with a septum and ClCH₂CH₂C₁ (2.5 mL) was added. Oxygen was added via active purge. The septum was replaced with a crimping cap and the vessel was sealed and stirred at the indicated temperature and time. The reaction mixture was filtered through a plug of silica and concentrated. The residue was chromatographed using 10% ethyl acetate/hexane to afford the product.

### Example 17: 3′,5′-Di-tert-3,6-methyl-[1,1′-biphenyl]-2,4,4′-triol (A2)

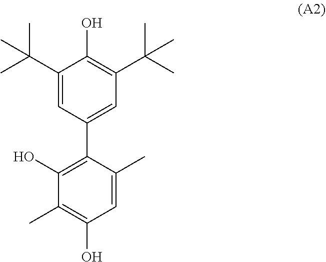 [SMILES: Cc1cc(O)c(C)c(O)c1-c1cc(C(C)(C)C)c(O)c(C(C)(C)C)c1]

Following general procedure B at 80° C. for 18 h, the product was obtained in 38% yield (15 mg, 0.044 mmol): ¹H NMR (500 MHz, CDCl₃) δ 7.03 (s, 2H), 6.34 (s, 1H), 5.29 (s, 1H), 5.09 (s, 1H), 4.66 (s, 1H), 2.16 (s, 3H), 2.01 (s, 3H), 1.44 (s, 18H); ¹³C NMR (125 MHz, CDCl₃) δ 153.5, 153.3, 152.0, 136.9, 134.8, 127.2, 126.0, 121.5, 108.2, 107.1, 34.5, 30.4, 20.2, 8.2; IR (film) 3536, 3525, 2959, 1727, 1416, 1234, 1080, 908, 734 cm¹; HRMS (ESI-TOF) m/z=341.2117 calc for C₂₄H₂₉O₃ [M−H]⁻, found 341.2115.

### Example 18: 3,3″,5,5″-Tetra-tert-butyl-2′,5′-dimethyl-[1,1′:3′,1″-terphenyl]-4,4′,4″,6′-tetraol (A11)

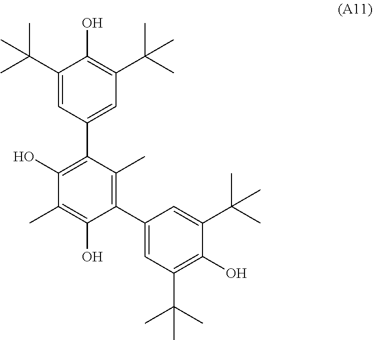 [SMILES: Cc1c(O)c(-c2cc(C(C)(C)C)c(O)c(C(C)(C)C)c2)c(C)c(-c2cc(C(C)(C)C)c(O)c(C(C)(C)C)c2)c1O]

Following general procedure B at 80° C. for 18 h, the product was obtained in a 29% yield (18 mg, 0.034 mmol): ¹H NMR (500 MHz, CDCl₃) δ 7.09 (s, 4H), 5.29 (s, 2H), 5.07 (s, 2H), 2.22 (s, 3H), 1.76 (s, 3H), 1.45 (s, 36H); ¹³C NMR (125 MHz, CDCl₃) δ 153.4, 151.2, 136.9, 132.7, 127.4, 126.5, 121.0, 107.1, 34.5, 30.4, 18.4, 8.7; IR (film) 3638, 3531, 2959, 1616, 1438, 1415, 1234, 1085, 909, 734 cm¹; HRMS (ESI-TOF) m/z=547.3787 calc for C₃₆H₅₁O₄ [M+H]⁺, found 547.3803.

### Example 19: 1-(4-Hydroxy-3,5-dimethylphenyl)naphthalene-2-ol (B1)

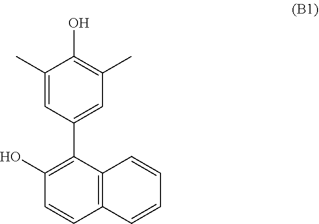 [SMILES: Cc1cc(-c2c(O)ccc3ccccc23)cc(C)c1O]

Following general procedure B at 80° C. for 18 h, the product was obtained in a 65% yield (20 mg, 0.076 mmol): ¹H NMR (500 MHz, CDCl₃) δ 7.80-7.76 (m, 2H), 7.45 (d, J=8.5 Hz, 1H), 7.36-7.29 (m, 2H), 7.25 (d, J=9.0 Hz, 1H), 7.03 (s, 2H), 5.24 (s, 1H), 4.79 (s, 1H), 2.23 (s, 6H); ¹³C NMR (125 MHz, CDCl₃) δ 152.4, 150.3, 133.6, 131.2, 129.1, 128.9, 128.0, 127.0, 126.3, 124.8, 124.3, 123.1, 121.9, 117.2, 16.0; IR (neat) 3638, 3531, 2959, 1616, 1438, 1415, 1234, 1085, 909, 734 cm⁻¹; FIRMS (ESI-TOF) m/z=264.1150 calc for C₈H₁₆O₂ [M]⁺, found 264.1148.

### Example 20: 1-(4-Hydroxy-3,5-dimethoxyphenyl)naphthalen-2-ol (B2)

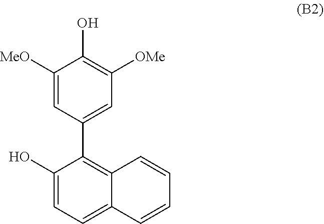 [SMILES: COc1cc(-c2c(O)ccc3ccccc23)cc(OC)c1O]

Following general procedure B at 80° C. for 24 h, product was obtained in 72% yield (20 mg, 0.067 mmol): ¹H NMR (500 MHz, CDCl₃) 7.82 (d, J=5.0 Hz, 1H), 7.80 (d, J=6.5 Hz, 1H), 7.47 (d, J=8.5 Hz, 1H), 7.39-7.34 (m, 2H), 7.27 (d, J=9.0 Hz, 1H), 6.64 (s, 2H), 5.70 (s, 1H), 5.30 (s, 1H), 3.90 (s, 6H); ¹³C NMR (125 MHz, CDCl₃) 150.3, 148.0, 134.7, 133.5, 129.4, 128.8, 128.0, 126.5, 124.6, 124.5, 123.3, 121.0, 117.2, 107.4, 56.4; IR (film) 3466, 1620, 1518, 1465, 1393, 1335, 1212, 1114, 817, 735, 634 cm¹; HRMS (ESI) m/z=297.1127 calc for C₁₈H₁₇O₄ [M+H]⁺, found 297.1132.

### Example 21: 1-(3,5-Di-tert-butyl-4-hydroxyphenol)naphthalene-2-7-diol (B4)

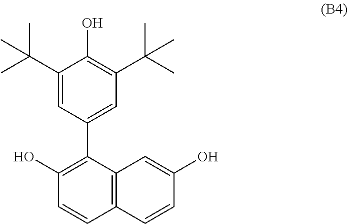 [SMILES: CC(C)(C)c1cc(-c2c(O)ccc3ccc(O)cc23)cc(C(C)(C)C)c1O]

Following general procedure B at 80° C. for 18 h, the product was obtained in a 88% yield (30 mg, 0.083 mmol): ¹H NMR (500 MHz, CDCl₃) δ 7.71 (d, J=2.0 Hz, 1H), 7.70 (d, J=3.0 Hz, 1H), 7.16 (s, 2H), 7.11 (d, J=9.0 Hz, 1H), 6.93 (dd, J=9.0 Hz, 2.0 Hz, 1H), 6.74 (d, J 2.0 Hz, 1H), 5.39 (s, 1H), 5.26 (s, 1H), 4.88 (s, 1H), 1.47 (s, 18H); ¹³C NMR (125 MHz, CDCl₃) δ 154.0, 151.1, 137.3, 135.2, 130.0, 128.9, 127.5, 124.5, 124.3, 120.7, 114.8, 114.6, 107.1, 34.5, 30.4; IR (film) 3498, 2959, 1625, 1516, 1434, 1172, 831, 738 cm⁻¹; HRMS (ESI-TOF) m/z=363.1960 calc for C₂₄H₂₇O₃ [M−H]⁻, found 363.1946.

### Example 22: General Procedure C for Oxidative Coupling of Phenols

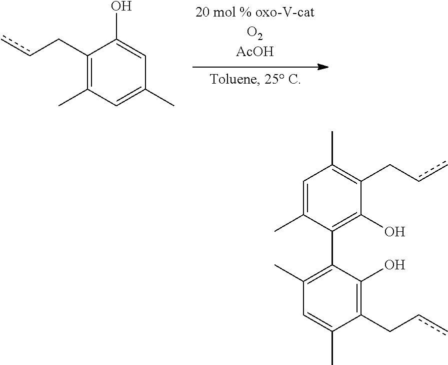

To a microwave vial was added phenol, 20 mol % oxovanadium catalyst, and acetic acid (6.25 equiv). The vial was sealed and toluene (0.5 M) was added. Oxygen was added via active purge. The mixture was stirred for the specified time at 25° C., and then concentrated. The residue was chromatographed to yield the homo-coupled product.

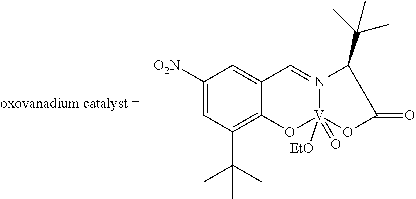

### Example 23: 3,3′Diallyl-4,4′,6,6′-tetramethyl-[1,1′-biphenyl]-2,2′-diol (A5)

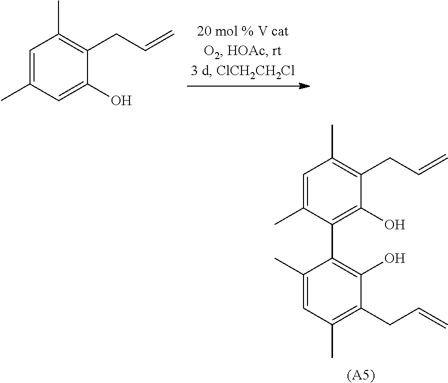

Following general procedure C, 2-allyl-3,5-dimethylphenol (0.2 mmol) after 3 d afforded the product as a white solid (18 mg, 0.06 mmol) in 56% yield: ¹H NMR (500 MHz, CDCl₃) δ 6.75 (s, 2H), 5.99-5.92 (m, 2H), 5.00-4.93 (m, 4H), 4.77 (s, 2H), 3.44-3.42 (m, 4H), 2.30 (s, 6H), 1.93 (s, 6H); ¹³C NMR (125 MHz, CDCl₃) δ 151.8, 138.6, 136.0, 135.9, 124.2, 121.9, 117.2, 114.4, 30.6, 19.4, 19.2; IR (film) 3524, 3077, 2922, 1637, 1564, 1457, 1302, 1258, 1191, 1144, 1110, 1050, 995, 909, and 851 cm⁻¹; HRMS (ESI-TOF) m/z=323.2011 calc for C₂₂H₂₇O₂ [M+H]⁺, found 323.2020.

### Example 24: 4,4’,6,6′-Tetramethyl-3,3′-dipropyl-[1,1′-diphenyl]2,2′-diol (A4)

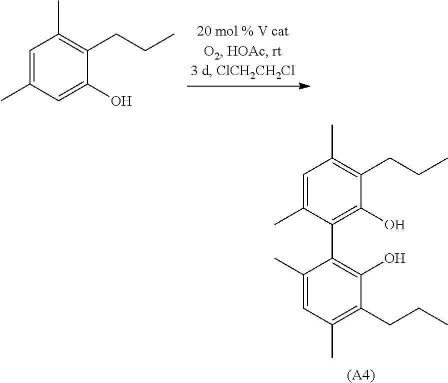

Following general procedure C, 3,5-dimethyl-2-propylphenol (0.2 mmol) after 3 d afforded the product as a white solid (26 mg, 0.08 mmol) in 80% yield: ¹H NMR (500 MHz, CDCl₃) δ 6.72 (s, 2H), 4.72 (s, 2H), 2.64-2.61 (m, 4H), 2.32 (s, 6H), 1.92 (s, 6H), 1.58-1.53 (m, 4H), 0.98-0.95 (m, 6H); ¹³C NMR (125 MHz, CDCl₃) δ 151.8, 138.0, 135.2, 125.2, 124.0, 117.1, 28.5, 22.4, 19.4, 19.2, 14.3; IR (film) 3524, 2959, 2871, 1616, 1563, 1453, 1394, 1296, 1260, 1205, 1146, 1103, 1039, 954, 850 cm⁻¹; HRMS (ESI-TOF) m/z=327.2324 calc for C₂₂H₃₁O₂ [M+H]⁺, found 327.2323.

### Example 25: 5,5′-(Ethane-1,2-diyl)bis(2-(tert-butyl)phenol) (C_{2/1}H)

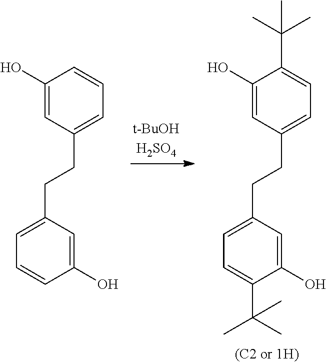 [SMILES: Oc1cccc(CCc2cccc(O)c2)c1 ; CC(C)(C)c1ccc(CCc2ccc(C(C)(C)C)c(O)c2)cc1O]

To a solution of 3,3′-(ethane-1,2-diyl)diphenol (166 mg, 0.77 mmol), which was prepared as set forth in Hata, Chem. Pharm. Bull 1979, 27, 984, in CH₂Cl₂ (4 mL, 0.2 M) at 0° C. was added tert-butanol (62 mg, 2.4 equiv) and conc H₂SO₄ (80 mg, 2.4 equiv). The mixture was allowed to warm to room temperature and stirred for 24 h. It was quenched with NaHCO₃(5 mL) and the organic layer was separated. The aqueous layer was extracted with CH₂Cl₂ (3×5 mL) and the combined organic fractions were dried over MgSO₄, concentrated and chromatographed (hexane/EtOAc, 8:1). The product was obtained in 57% yield as a white solid (0.1570 g, 0.4809 mmol): ¹H NMR (500 MHz, CDCl₃) δ 7.19 (d, J=8.0 Hz, 2H), 6.74 (dd, J=8.0 Hz, 1.5 Hz, 2H), 6.52 (d, J=1.5 Hz, 2H), 4.71 (s, 2H), 2.81 (s, 4H), 1.41 (s, 18H); ¹³C NMR (125 MHz, CDCl₃) δ 154.5, 141.2, 133.9, 127.2, 120.4, 116.7, 36.9, 34.5, 29.9; IR (neat) 3513, 2955, 2869, 1709, 1615, 1483, 1468, 1416, 1390, 1375, 1362, 1297, 1260, 1201, 1184, 1137, 1079, 1043, 858, 816, 573 cm⁻¹; HRMS (ESI-TOF) m/z=327.2324 calc for C₂₂H₃₁O₂ [M+H]⁺, found 327.2296.

### Example 26: General Procedure A for Grignard Reactions

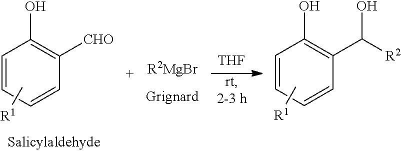

To a solution of salicylaldehyde (1 mmol) in anhydrous THF (0.2 M) was slowly added a solution of Grignard reagent (1 M in diethyl ether) (4.5 mmol) at 0° C. The mixture was stirred for 2-3 h at room temperature. The reaction mixture was cooled to 0° C. and quenched via drop-wise addition of satd. NH₄Cl (5 mL). After stirring at room temperature for 10 min, the product was extracted with diethyl ether (5×20 mL). The combined organic fractions were dried over Na₂SO₄, concentrated, and chromatographed using 30% ethyl acetate/hexane to afford the product.

### Example 27: 2-hydroxy-2-(3-methoxyphenyl)cyclohexan-1-one

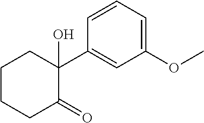 [SMILES: COc1cccc(C2(O)CCCCC2=O)c1]

Following the general procedure A, the product was obtained as a clear yellow liquid (266 mg, 1.2 mmol) in a 45% yield: ¹H NMR (500 MHz, CDCl₃) δ 7.30 (t, J=7.8 Hz, 1H), 6.89-6.85 (m, 3H), 4.47 (s, 1H), 3.80 (s, 3H), 2.98-2.95 (m, 1H), 2.56-2.51 (m, 1H), 2.45 (m, 1H), 2.07-2.04 (m, 1H), 1.88-1.70 (m, 4H); ¹³C NMR (125 MHz, CDCl₃) δ 212.7, 160.3, 141.6, 130.3, 118.8, 113.6, 112.7, 80.2, 55.5, 39.11, 39.05, 28.4, 23.2; IR (neat) 3341, 2937, 1676, 1599, 1585, 1487, 1454, 1433, 1317, 1256, 1153, 1040, 872, 784, 735, 699, 564, 465 cm⁻¹; HRMS (ESI-TOF) m/z=220.1099 calc for C₁₃H₁₆O₃ [M]⁺, found 220.1095.

### Example 28: 5,5′-(ethane-1,2-diyl)bis(2-(1-hydroxyethyl)phenol)

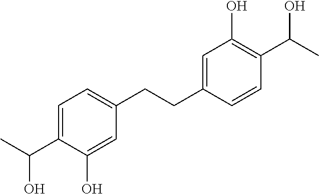 [SMILES: CC(O)c1ccc(CCc2ccc(C(C)O)c(O)c2)cc1O]

Following general procedure A, the product was obtained as a white solid (39.1 mg, 0.129 mmol) in a 70% yield: ¹H NMR (500 MHz, acetone-D₆) δ 8.57 (s, 2H), 7.07 (d, J=8.0 Hz, 2H), 6.69-6.68 (m, 4H), 5.09 (s, 1H), 5.08 (s, 1H), 4.84 (m, 2H), 2.79 (s, 4H), 1.45 (s, 3H), 1.44 (s, 3H); ¹³C NMR (125 MHz, Acetone-D₆) δ 155.0, 141.9, 128.5, 126.1, 119.4, 115.9, 68.1, 37.1, 23.7.

### Example 29: 5,5′-(ethane-1,2-diyl)bis(2-(2-hydroxypropan-2-yl)phenol)

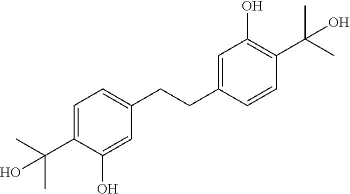 [SMILES: CC(C)(O)c1ccc(CCc2ccc(C(C)(C)O)c(O)c2)cc1O]

Following general procedure A, the product was obtained as a light brown solid (31.7 mg, 0.096 mmol) in a 56% yield: ¹H NMR (500 MHz, CDCl₃) δ 8.77 (s, 2H), 7.06 (s, 2H), 6.71 (dd, J=5.8, 2.5 Hz, 2H), 6.66-6.65 (m, 4H), 5.28 (s, 1H), 5.27 (s, 1H), 3.08-3.04 (m, 4H), 1.68 (s, 12H).

### Example 30: General Procedure B for Friedel-Crafts Electrophilic Aromatic Substitution

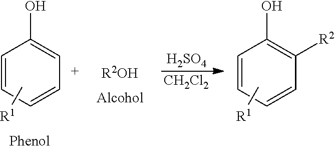

To a solution of the phenol (1 equiv) in dichloromethane (0.2 M) at 0° C. was added the alcohol (2.2 equiv) and H₂SO₄ (2 equiv). The mixture was stirred for 18 h with monitoring via TLC. When the reaction was finished, it was quenched with a saturated solution of NaHCO₃ (10 mL), extracted with dichloromethane (5×15 mL) and ethyl acetate (5×15 mL), and washed with brine (5×10 mL). The combined organic fractions were dried over Na₂SO₄, concentrated, and chromatographed using 10% ethyl acetate/hexane to afford the product.

### Example 31: 5,5′-methylenebis(2-(tert-butyl)phenol) (3N)

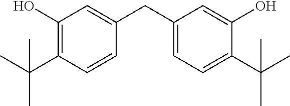 [SMILES: CC(C)(C)c1ccc(Cc2ccc(C(C)(C)C)c(O)c2)cc1O]

Following general procedure B, the product was obtained as a light brown solid (8.9 mg, 0.03 mmol) in a 12% yield: ¹H NMR (500 MHz, CDCl₃) δ 7.16 (d, J=8 Hz, 2H), 6.70 (dd, J=8, 1.5 Hz, 2H), 6.43 (d, J=1.5 Hz, 2H), 4.63 (s, 2H), 3.77 (s, 2H), 1.37 (s, 18H); ¹³C NMR (125 MHz, CDCl₃) δ 154.4, 140.2, 134.1, 127.3, 121.3, 117.3, 40.5, 34.5, 29.9; IR (neat) 3521, 2956, 2912, 2870, 1614, 1573, 1514, 1483, 1414, 1391, 1362, 1333, 1296, 1262, 1185, 1136, 1080, 977, 811, 762, 734, 502 cm⁻¹; HRMS (EI-TOF) m/z=312.2089 calc for C₂₁H₂₈O₂ [M]⁺, found 312.2092.

### Example 32: bis(4-(tert-butyl)-3-methoxyphenyl)methane

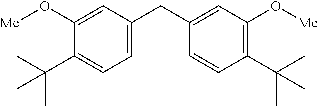 [SMILES: COc1cc(Cc2ccc(C(C)(C)C)c(OC)c2)ccc1C(C)(C)C]

Following general procedure B, the product was obtained as a light yellow liquid (13.2 mg, 0.039 mmol) in a 18% yield: ¹H NMR (500 MHz, CDCl₃) δ 7.18 (d, J=8.2 Hz, 2H), 6.73 (m, 4H), 3.91 (s, 2H), 3.80 (s, 6H), 1.36 (s, 18H).

### Example 33: 5-(3-(tert-butoxy)phenethyl)-2-(tert-butyl)phenol

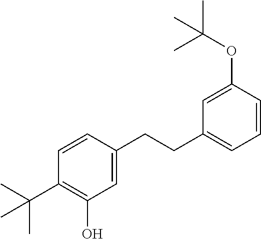 [SMILES: CC(C)(C)Oc1cccc(CCc2ccc(C(C)(C)C)c(O)c2)c1]

Following general procedure B, the product was obtained as a white solid (2.1 mg, 0.007 mmol) in a 3% yield: ¹H NMR (500 MHz, CDCl₃) δ 8.07 (s, 1H), 7.16 (t, J=8.0 Hz, 1H), 7.08 (d, J=8.0 Hz, 1H), 6.93 (d, J=7.4 Hz, 1H), 6.79-6.78 (m, 2H), 6.67 (s, 1H), 6.61 (d, J=8.0 Hz, 1H), 2.79-2.78 (m, 4H), 1.37 (s, 9H), 1.27 (s, 9H).

### Example 34: 5,5′-(ethane-1,2-diyl)bis(2-(3-ethylpentan-3-yl)phenol)

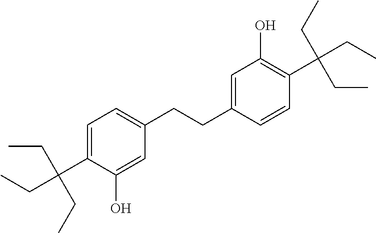 [SMILES: CCC(CC)(CC)c1ccc(CCc2ccc(C(CC)(CC)CC)c(O)c2)cc1O]

Following general procedure B, the product was obtained as a light yellow liquid (8.8 mg, 0.021 mmol) in a 9% yield: ¹H NMR (500 MHz, CDCl₃) δ 7.04 (d, J=8.0 Hz, 2H), 6.66 (dd, J=6.25, 1.7 Hz, 4H), 6.37-6.36 (m, 2H), 4.52 (s, 2H), 2.78 (s, 4H), 1.80 (q, J=7.5 Hz, 12H), 0.64 (t, J=7.5 Hz, 18H).

### Example 35: 5,5′-(ethane-1,2-diyl)bis(24-adamantan-1-yl)phenol)

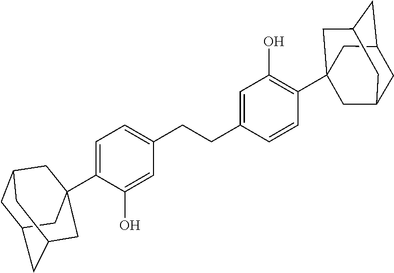 [SMILES: Oc1cc(CCc2ccc(C34CC5CC(CC(C5)C3)C4)c(O)c2)ccc1C12CC3CC(CC(C3)C1)C2]

Following general procedure B, the product was obtained as a white solid (14.1 mg, 0.029 mmol) in a 21% yield (˜15% impure): ¹H NMR (500 MHz, CDCl₃) δ 7.13 (d, J=8.0 Hz, 2H), 6.77 (dd, J=6.5, 1.9 Hz, 2H), 6.50 (m, 2H), 4.68 (s, 2H), 2.81 (s, 4H), 2.12 (m, 12H), 1.78 (s, 18H).

### Example 36: 2-(tert-butyl)-5-(3-hydroxyphenethyl)phenol (111-3)

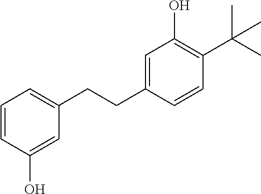 [SMILES: CC(C)(C)c1ccc(CCc2cccc(O)c2)cc1O]

Following general procedure B, the product was obtained as a dark yellow solid (46.4 mg, 0.172 mmol) in a 18% yield: ¹H NMR (500 MHz, CDCl₃) δ 7.04-7.00 (m, 3H), 6.87 (dd, J=8.0, 2.0 Hz, 1H), 6.75 (dd, J=6.5, 2.0 Hz, 2H), 6.58 (d, J=8.0 Hz, 1H), 2.81 (s, 4H), 1.39 (s, 9H).

### Example 37: 3,3′-(ethane-1,2-diyl)bis(4-(tert-butyl)phenol)

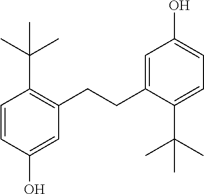 [SMILES: CC(C)(C)c1ccc(O)cc1CCc1cc(O)ccc1C(C)(C)C]

Following general procedure B, the product was obtained as a brown solid (660 mg, 2.02 mmol) in a 47% yield: ¹H NMR (500 MHz, acetone-D₆) δ 8.06 (s, 2H), 7.09 (d, J=8.0 Hz, 2H), 6.70 (d, J=2.0 Hz, 2H), 6.64 (dd, J=8.0, 2.0 Hz, 2H), 2.73 (s, 4H), 1.37 (s, 9H).

### Example 38: 2,4-di-tert-butyl-5-(4-(tert-butyl)-3-hydroxyphenethyl)phenol

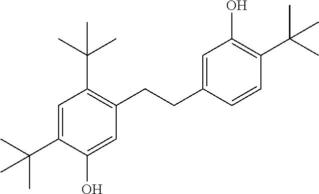 [SMILES: CC(C)(C)c1ccc(CCc2cc(O)c(C(C)(C)C)cc2C(C)(C)C)cc1O]

Following general procedure B, the product was obtained as a brown gel (37.8 mg, 0.099 mmol) in a 11% yield: ¹H NMR (500 MHz, CDCl₃) δ 7.06 (s, 1H), 6.97 (d, J=2.0 Hz, 2H), 6.89 (dd, J=8.0, 2.0 Hz, 1H), 6.59 (d, J=8.0 Hz, 1H), 5.03 (s, 1H), 4.64 (s, 1H), 2.81 (s, 4H), 1.43 (s, 18H), 1.40 (s, 9H); ¹³C NMR (125 MHz, CDCl₃) δ 152.1, 151.8, 135.7, 135.6, 134.0, 132.5, 127.1, 126.6, 124.8, 116.3, 38.0, 37.6, 34.4, 34.2, 30.3, 29.6.

### Example 39: 5,5′-(ethane-1,2-diyl)bis(2,4-di-tert-butylphenol)

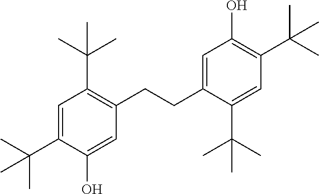 [SMILES: CC(C)(C)c1cc(C(C)(C)C)c(CCc2cc(O)c(C(C)(C)C)cc2C(C)(C)C)cc1O]

Following general procedure B, the product was obtained as a yellow solid (7.3 mg, 0.017 mmol) in a 2% yield: ¹H NMR (500 MHz, CDCl₃) δ 6.98 (s, 4H), 5.02 (s, 2H), 2.82 (s, 4H), 1.43 (s, 36H); ¹³C NMR (125 MHz, CDCl₃) δ 151.7, 135.6, 132.7, 124.8, 37.9, 34.2, 30.3.

### Example 40: 4,4′-di-tert-butyl-[1,1′-biphenyl]-3,3′-diol

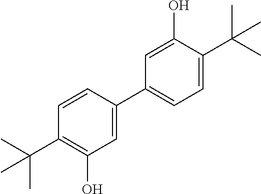 [SMILES: CC(C)(C)c1ccc(-c2ccc(C(C)(C)C)c(O)c2)cc1O]

Following general procedure B, the product was obtained as a white solid (2.8 mg, 0.009 mmol) in a 3% yield: ¹H NMR (500 MHz, acetone-D₆) δ 8.33 (s, 2H), 7.23 (d, J=8.0 Hz, 2H), 7.04 (d, J=1.8 Hz), 6.96 (dd, J=8.0, 1.8 Hz, 2H), 1.40 (s, 18H).

### Example 41: 5,5′-(propane-1,3-diyl)bis(2-(tert-butyl)phenol)

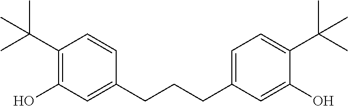 [SMILES: CC(C)(C)c1ccc(CCCc2ccc(C(C)(C)C)c(O)c2)cc1O]

Following general procedure B, the product was obtained as a yellow liquid (5.6 mg, 0.016 mmol) in a 13% yield: ¹H NMR (500 MHz, CDCl₃) δ 7.16 (d, J=7.9 Hz, 2H), 6.70 (d, J=7.9 Hz, 2H), 6.49 (s, 2H), 4.67 (s, 2H), 2.56 (t, J=7.6 Hz, 4H), 1.91 (p, J=7.6 Hz, 2H), 1.39 (s, 18H).

### Example 42: 2,2′-(ethane-1,2-diyl)bis(4-(tert-butyl)phenol)

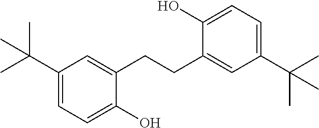 [SMILES: CC(C)(C)c1ccc(O)c(CCc2cc(C(C)(C)C)ccc2O)c1]

Following general procedure B, the product was obtained as a white solid (3.0 mg, 0.009 mmol) in a 7% yield: ¹H NMR (500 MHz, CDCl₃) δ 7.16 (m, 4H), 6.80 (d, J=8.7 Hz, 2H), 5.98 (s, 2H), 2.85 (s, 4H), 1.30 (s, 18H).

### Example 43: 4,4′-(ethane-1,2-diyl)bis(2-(tert-butyl)phenol)

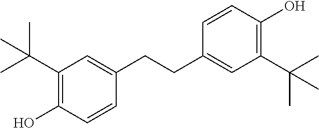 [SMILES: CC(C)(C)c1cc(CCc2ccc(O)c(C(C)(C)C)c2)ccc1O]

Following general procedure B, the product was obtained as a light yellow solid (9.3 mg, 0.029 mmol) in a 6% yield: ¹H NMR (500 MHz, CDCl₃) δ 7.02 (d, J=2.2 Hz, 2H), 6.87 (dd, J=6.0, 2.2 Hz, 2H), 6.58 (d, J=6.0 Hz, 2H), 4.69 (s, 2H), 2.80 (s, 4H), 1.39 (s, 18H).

### Example 44: General Procedure C Hydrogenation of Benzaldehydes and Benzylic Alcohols

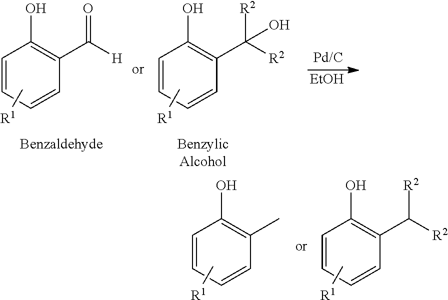

To a round bottom flask with benzaldehyde or benzylic alcohol (1 equiv) and palladium (10% on carbon, 0.9 equiv) was added anhydrous ethanol (0.1 M). Hydrogen was added via active purge and the mixture was allowed to stir for 18 h. After completion of the reaction, the flask was purged with argon and filtered through a bed of celite. The solvent was evaporated and product was chromatographed using 10% ethyl acetate/hexane to afford the product.

### Example 45: 5,5′-(ethane-1,2-diyl)bis(2-methylphenol)

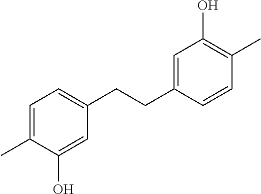

Following general procedure C, the product was obtained as a white solid (14.9 mg, 0.061 mmol) in a 68% yield: ¹H NMR (500 MHz, acetone-D₆) δ 6.95 (d, J=7.5 Hz, 2H), 6.67 (d, J=1.5 Hz, 2H), 6.59 (dd, J=7.5, 1.5 Hz, 2H), 2.74 (s, 4H), 2.14 (s, 6H).

### Example 46: 5,5′-(ethane-1,2-diyl)bis(2-isopropylphenol)

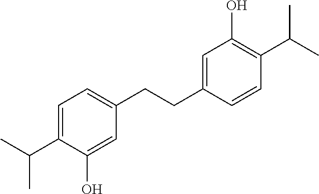 [SMILES: CC(C)c1ccc(CCc2ccc(C(C)C)c(O)c2)cc1O]

Following general procedure C, the product was obtained as a white solid (2.3 mg, 0.008 mmol) in a 26% yield: ¹H NMR (500 MHz, CDCl₃) δ 7.09 (d, J=7.8 Hz, 2H), 7.76 (d, J=7.8 Hz, 2H), 6.59 (s, 2H), 4.60 (s, 2H), 3.15 (p, J=7.4 Hz, 2H), 2.80 (s, 4H), 1.24 (s, 6H), 1.23 (s, 6H).

### Example 47: 4,4′-(ethane-1,2-diyl)bis(2-hydroxybenzaldehyde)

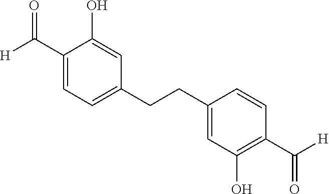 [SMILES: O=Cc1ccc(CCc2ccc(C=O)c(O)c2)cc1O]

To a solution of 3,3′-(ethane-1,2-diyl)diphenol (700 mg, 3.27 mmol) and anhydrous MgCl₂ (1244 mg, 13.07 mmol) in THF (17 mL, 0.2 M) was added triethylamine (1.83 mL, 13.07 mmol). The mixture was stirred for 15 min after which paraformaldehyde (784.2 mg, 26.14 mmol) was added. After heating at reflux for 18 h, the reaction was quenched with 3 M HCl (aq) (50 mL), extracted with diethyl ether (5×35 mL), and washed with H₂O (3×30 mL) and saturated solution of NaCl (3×30 mL). The combined organic fractions were dried over Na₂SO₄, concentrated, and recrystallized using acetone. The product was obtained as a white solid (400.1 mg, 1.48 mmol) in a 45% yield: ¹H NMR (500 MHz, CDCl₃) δ 9.83 (s, 2H), 7.45 (d, J=7.9 Hz, 2H), 6.80 (m, 4H), 2.94 (s, 4H); ¹³C NMR (125 MHz, CDCl₃) δ 196.0, 162.0, 151.4, 133.9, 120.5, 119.3, 117.3, 37.3.

### Example 48: (E)-1,3-bis(3-methoxyphenyl)prop-2-en-1-one

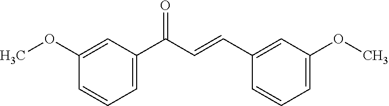

To a flame dried round bottom flask was added 3-methoxybenzaldehyde (2.44 mL, 20 mmol), 1-(3-methoxyphenyl)ethan-1-one (2.74 mL, 20 mmol), and BF₃.OEt₂ (1.24 mL, 10 mmol). The mixture was stirred for 4 h, quenched with H₂O (30 mL), and extracted with ethyl acetate (5×40 mL). The solvent was evaporated and product chromatographed (hexane/EtOAc, 10:1). The product was obtained as a brown liquid (943 mg, 3.52 mmol) in a 18% yield: ¹H NMR (500 MHz, CDCl₃) δ 7.76 (d, J=15.7 Hz, 1H), 7.59 (dt, J=7.6, 1.1 Hz, 1H), 7.54 (dd, J=2.1, 1.0 Hz, 1H), 7.48 (d, J=15.7 Hz, 1H), 7.38 (t, J=8.0 Hz, 1H), 7.31 (t, J=7.8 Hz, 1H), 7.22 (d, J=7.7 Hz, 1H), 7.14 (t, J=2.1 Hz, 1H), 7.11 (ddd, J=9, 2.7, 0.8 Hz, 1H) 6.94 (ddd, J=8.8, 2.5, 0.5 Hz, 1H), 3.85 (s, 3H), 3.82 (s, 3H); ¹³C NMR (125 MHz, CDCl₃) δ 190.1, 160.0, 159.9, 144.7, 139.6, 136.3, 130.0, 129.6, 122.4, 121.12, 121.07, 119.3, 116.3, 113.5, 113.0, 55.5, 55.3; IR (neat) 3002, 2938, 2835, 1662, 1578, 1485, 1452, 1430, 1314, 1286, 1257, 1195, 1165, 1088, 1028, 993, 979, 878, 847, 776, 728, 683, 568, 549, 502 cm⁻¹; HRMS (EI-TOF) m/z=268.1099 calc for C₁₇H₁₆O₃ [M⁺], found 268.1101.

### Example 49: 1,3-bis(3-methoxyphenyl)propan-1-ol

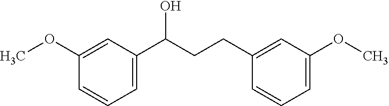

To a flame dried round bottom flask, was added Pd(OAc)₂ (19.67 mg, 0.09) and NaBH₄ (33.13 mg, 0.88 mmol). (E)-1,3-Bis(3-methoxyphenyl)prop-2-en-1-one (235 mg, 0.88 mmol) was dissolved in CHCl₃ (1 mL, 0.9 M) and the solution added to the round bottom flask with the solids. With a balloon as an outlet for H₂ gas produced, MeOH (4 mL, 0.23 M) was slowly added to the flask. After reaction completion, the mixture was filtered through celite. The solvent was evaporated and the product was chromatographed (hexane/EtOAc, 10:1). The product was obtained as a clear liquid (31.6 mg, 0.12 mmol) in a 13% yield: ¹H NMR (500 MHz, CDCl₃) δ 7.27 (t, J=8 Hz, 1H), 7.21 (t, J=7.7 Hz, 1H), 6.94-6.92 (m, 2H), 6.84-6.81 (m, 1H), 6.80 (d, J=7.5 Hz, 1H), 6.76-6.73 (m, 2H), 4.66 (dd, J=6.4, 2.2 Hz, 1H), 3.81 (s, 3H), 3.79 (s, 3H), 2.77-2.63 (m, 2H), 2.15-1.99 (m, 2H); ¹³C NMR (125 MHz, CDCl₃) δ 159.8, 159.7, 147.8, 143.9, 129.1, 128.9, 120.5, 118.0, 113.9, 112.1, 111.3, 111.0, 72.5, 54.4, 54.4, 41.2, 31.9; IR (neat) 3417, 2939, 2835, 1600, 1584, 1487, 1454, 1434, 1316, 1256, 1191, 1151, 1039, 995, 938, 867, 780, 725, 696, 561, 502, 471 cm⁻¹; HRMS (ESI-TOF) m/z=272.1412 calc for C₁₇H₂₀O₃ [M]⁺, found 272.1404.

### Example 50: 3,3′-(ethane-1,2-diyl)diphenol

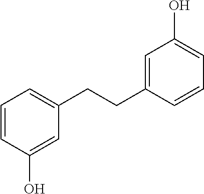 [SMILES: Oc1cccc(CCc2cccc(O)c2)c1]

To a solution of 1,2-bis(3-methoxyphenyl)ethane (0.060 mg, 2.78 mmol) in anhydrous dichloromethane (11 mL, 0.2 M) at −78° C. was added BBr₃ (1 M solution in dichloromethane) (6.4 mL, 6.40 mmol) dropwise under an argon flow. After stirring 30 min, the mixture was slowly warmed to room temperature and stirred for 12 h. The reaction was quenched via dropwise addition of H₂O, and extracted using ethyl acetate (5×30 mL). The combined organic fractions were dried over Na₂SO₄, concentrated, and chromatographed (hexane/EtOAc, 10:1). The demethylated product was obtained as a white solid (535.6 mg, 2.5 mmol) in a 90% yield: ¹H NMR (500 MHz, acetone-D₆) δ 8.11 (s, 2H), 7.08 (t, J=7.8 Hz, 2H), 6.72-6.69 (m, 4H), 6.66-6.64 (m, 2H), 2.81 (s, 4H); ¹³C NMR (125 MHz, Acetone-D₆) δ 157.5, 143.6, 129.3, 119.7, 115.5, 112.9, 37.6; IR (neat) 3328, 2927, 1589, 1491, 1455, 1254, 1155, 939, 865, 783, 693 cm⁻¹; HRMS (ESI-TOF) m/z=214.0994 calc for C₁₄H₁₄O₂ [M]⁺, found 214.0991.

### Example 51: bis(3-methoxyphenyl)methane

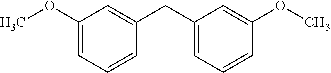

To a round bottom flask with bis(3-methoxyphenyl)methanol (200 mg, 0.82 mmol) and palladium, 10% on carbon (80 mg, 0.75 mmol) was added anhydrous ethanol (6 mL, 0.14 M). The flask was backfilled with hydrogen gas three times and allowed to stir for 18 h. After completion of the reaction, the flask was purged with argon and filtered through a bed of celite. The solvent was evaporated and the product was chromatographed (hexane/EtOAc, 10:1). The product was obtained as a clear liquid (117.7 mg, 0.52 mmol) in a 63% yield: ¹H NMR (500 MHz, CDCl₃) δ 7.24 (m, 2H), 6.83 (d, J=7.6 Hz, 2H), 6.79-6.78 (m, 4H), 3.96 (s, 2H), 3.80 (s, 6H); ¹³C NMR (125 MHz, CDCl₃) δ 159.9, 142.6, 129.5, 121.5, 114.9, 111.5, 55.3, 42.1; IR (neat) 3001, 2939, 2835, 1596, 1583, 1487, 1464, 1453, 1434, 1310, 1280, 1256, 1190, 1149, 1089, 1044, 996, 946, 873, 774, 774, 737, 691, 572, 554 cm⁻¹; HRMS (ESI-TOF) m/z=228.1150 calc for C₈H₁₆O₂ [M]⁺, found 228.1157.

### Example 52: 1,1′-(methylenebis(2-hydroxy-4,1-phenylene))bis(ethan-1-one)

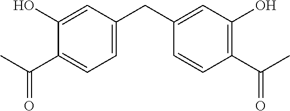 [SMILES: CC(=O)c1ccc(Cc2ccc(C(C)=O)c(O)c2)cc1O]

To a solution of bis(3-methoxyphenyl)methane (100 mg, 0.44 mmol) in anhydrous toluene (1.1 mL, 0.4 M) was added TiCl₄ (0.106 mL, 0.964 mmol) slowly. The mixture was stirred until gas evolution ceased. Acyl chloride (0.094 mL, 1.31 mmol) was slowly added and the solution stirred for 15 min. The solution was brought to 100° C. and stirred for 1 h. The mixture was quenched via drop wise addition of H₂O (5 mL), and then extracted with dichloromethane (5×10 mL). The combined organic fractions were dried over Na₂SO₄, concentrated, and chromatographed (hexane/EtOAc, 10:1). The product was obtained as a brown liquid (31.2 mg, 0.10 mmol) in a 25% yield: ¹H NMR (500 MHz, acetone-D₆) δ 12.29 (s, 2H), 7.82 (d, J=8.0 Hz, 2H), 6.83 (d, J=8.5 Hz, 2H), 6.81 (s, 2H), 3.97 (s, 2H), 2.60 (s, 6H); ¹³C NMR (125 MHz, CDCl₃) δ 205.7, 163.5, 150.6, 132.6, 120.8, 119.2, 118.9, 42.5, 23.4; IR (neat) 1634, 1620, 1569, 1504, 1420, 1363, 1322, 1301, 1281, 1245, 1221, 1165, 1148, 1023, 983, 959, 822, 794, 759, 750, 704, 602 cm¹; HRMS (ESI-TOF) m/z=284.1049 calc for C₁₇H₁₆₀₄[M]⁺, found 284.1048.

### Example 53: 4,4′-methylenebis(2-hydroxybenzaldehyde)

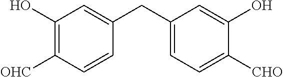 [SMILES: O=Cc1ccc(Cc2ccc(C=O)c(O)c2)cc1O]

To a solution of 3,3′-methylenediphenol (40 mg, 0.200 mmol) and anhydrous MgCl₂ (38 mg, 0.40 mmol) in THF (1 mL, 0.2 M) was added triethylamine (0.06 mL, 0.40 mmol). The mixture was stirred for 15 min after which paraformaldehyde (48 mg, 1.60 mmol) was added and the mixture heated at reflux for 18 h. The reaction was quenched with 3 M HCl (aq) (20 mL), extracted with diethyl ether (5×15 mL), and washed with H₂O (3×20 mL) and a saturated solution of NaCl (3×20 mL). The combined organic fractions were dried over Na₂SO₄, concentrated, and chromatographed (hexane/EtOAc, 10:1). The product was obtained as a white solid (10.6 mg, 0.04 mmol) in a 21% yield: ¹H NMR (500 MHz, acetone-D₆) δ 11.03 (s, 2H), 9.97 (s, 2H), 7.71 (d, J=7.9 Hz, 2H), 7.00 (d, J=7.9 Hz, 2H), 6.90 (s, 2H), 4.07 (s, 2H); ¹³C NMR (125 MHz, Acetone-D₆) 6196.7, 161.6, 150.2, 134.1, 120.9, 119.6, 117.3, 41.8; IR (neat) 3076, 2856, 1644, 1622, 1569, 1498, 1450, 1410, 1381, 1344, 1321, 1269, 1229, 1205, 1155, 1131, 983, 926, 888, 805, 733, 709, 637, 610, 598, 559, 475 cm⁻¹; FIRMS (ESI-TOF) m/z=256.0736 calc for C₁₄H₁₂O₄ [M]⁺, found 256.0722.

### Example 54: 2-hydroxy-4-(3-hydroxybenzyl)benzaldehyde

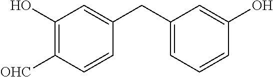 [SMILES: O=Cc1ccc(Cc2cccc(O)c2)cc1O]

To a solution of 3,3′-methylenediphenol (40 mg, 0.200 mmol) and anhydrous MgCl₂ (38 mg, 0.40 mmol) in THF (1 mL, 0.2 M) was added triethylamine (0.06 mL, 0.40 mmol). The mixture was stirred for 15 min after which paraformaldehyde (48 mg, 1.60 mmol) was added and the mixture heated at reflux for 18 h. The reaction was quenched with 3 M HCl (aq) (20 mL), extracted with diethyl ether (5×15 mL), and washed with H₂O (3×20 mL) and a saturated solution of NaCl (3×20 mL). The combined organic fractions were dried over Na₂SO₄, concentrated, and chromatographed (hexane/EtOAc, 10:1). The product was obtained as a light red solid (12.3 mg, 0.05 mmol) in a 27% yield: ¹H NMR (500 MHz, acetone-D₆) δ 9.95 (s, 1H), 7.67 (d, J=7.9 Hz, 1H), 7.12 (t, J=7.8 Hz, 1H), 6.95 (d, J=7.3 Hz, 1H), 6.84 (s, 1H), 6.74-6.68 (m, 3H), 3.93 (s, 2H); ¹³C NMR (125 MHz, CDCl₃) δ 195.8, 161.8, 155.7, 151.2, 141.0, 133.7, 129.8, 121.5, 120.7, 119.1, 117.7, 115.9, 113.5, 42.0; IR (neat) 3365, 2925, 2852, 1651, 1627, 1589, 1569, 1502, 1490, 1454, 1371, 1322, 1279, 1227, 1206, 1158, 1128, 1081, 1000, 975, 878, 809, 786, 749, 697, 674, 622, 557, 469 cm⁻¹; HRMS (EI-TOF) m/z=228.0786 calc for C₁₄H₁₂O₃ [M]⁺, found 228.0779.

### Example 55: 3,3′-(ethane-1,2-diyl)bis(2-hydroxybenzaldehyde)

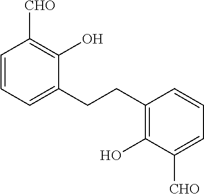 [SMILES: O=Cc1cccc(CCc2cccc(C=O)c2O)c1O]

To a solution of 3,3′-methylenediphenol (50 mg, 0.233 mmol) and anhydrous MgCl₂ (44.4 mg, 0.467 mmol) in THF (1.2 mL, 0.2 M) was added triethylamine (0.07 mL, 0.467 mmol). The mixture was stirred for 15 min after which paraformaldehyde (56 mg, 1.866 mmol)) was added and the mixture heated at reflux for 18 h. The reaction was quenched with 3 M HCl (aq) (20 mL), extracted with diethyl ether (5×15 mL), and washed with H₂O (3×20 mL) and saturated solution of NaCl (3×20 mL). The combined organic fractions were dried over Na₂SO₄, concentrated, and chromatographed (hexane/EtOAc, 10:1). The product was obtained as a white solid (4.2 mg, 0.02 mmol) in a 7% yield: ¹H NMR (500 MHz, CDCl₃) δ 11.32 (d, J=0.5 Hz, 2H), 9.89 (s, 2H), 7.42 (dd, J=7.5, 1.7 Hz, 2H), 7.33 (dd, J=7.5, 1.5 Hz, 2H), 6.91 (t, J=7.5 Hz, 2H), 3.00 (s, 4H); ¹³C NMR (125 MHz, CDCl₃) δ 196.8, 159.8, 137.4, 131.8, 130.2, 120.2, 119.4, 28.9; IR (neat) 2928, 2845, 1645, 1615, 1484, 1443, 1385, 1352, 1325, 1265, 1239, 1218, 1153, 1076, 978, 853, 792, 756, 733, 696, 645, 464 cm¹; HRMS (ESI-TOF) m/z=270.0892 calc for C₁₆H₁₄O₄ [M]⁺, found 270.0908.

### Example 56: ethane-1,2-diylbis(4,1-phenylene) diacetate

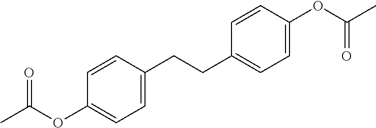 [SMILES: CC(=O)Oc1ccc(CCc2ccc(OC(C)=O)cc2)cc1]

To a solution of 1,2-bis(4-methoxyphenyl)ethane (50 mg, 0.21 mmol) in anhydrous toluene (0.5 mL, 0.4 M) was added TiCl₄ (0.05 mL, 0.45 mmol) slowly. The mixture was stirred until gas evolution ceased. Acyl chloride (0.03 mL, 0.45 mmol) was slowly added and the solution stirred for 15 min. The solution was brought to 100° C. and stirred for 1 h. The mixture was quenched via drop-wise addition of H₂O (5 mL), and then extracted with dichloromethane (5×10 mL). The combined organic fractions were dried over Na₂SO₄, concentrated, and chromatographed (hexane/EtOAc, 10:1). The product was obtained as a brown liquid (29.4 mg, 0.10 mmol) in a 48% yield: ¹H NMR (500 MHz, CDCl₃) δ 7.85 (d, J=8.2 Hz, 4H), 7.29 (d, J=8.0 Hz, 4H), 2.51 (s, 6H), 2.37 (s, 4H); ¹³C NMR (125 MHz, CDCl₃) δ 196.5, 143.4, 134.9, 129.0, 128.2, 25.6, 20.5; IR (neat) 2922, 1679, 1605, 1573, 1511, 1429, 1405, 1356, 1265, 1211, 1181, 1112, 1074, 1037, 1018, 953, 814, 713, 673, 637, 591, 567, 460 cm¹; HRMS (ESI-TOF) m/z=299.1283 calc for C₁₄H₁₉O₄ [M+H]⁺, found 299.1277.

### Example 57: 1,1′-(ethane-1,2-diylbis(6-hydroxy-3,1-phenylene))bis(ethan-1-one)

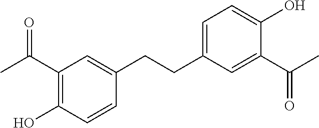 [SMILES: CC(=O)c1cc(CCc2ccc(O)c(C(C)=O)c2)ccc1O]

To a solution of 1,2-bis(4-methoxyphenyl)ethane (50 mg, 0.21 mmol) in anhydrous toluene (0.5 mL, 0.4 M) was added TiCl₄ (0.05 mL, 0.45 mmol) slowly. The mixture was stirred until gas evolution ceased. Acyl chloride (0.03 mL, 0.45 mmol) was slowly added and the solution stirred for 15 min. The solution was brought to 100° C. and stirred for 1 h. The mixture was quenched via drop-wise addition of H₂O (5 mL), and then extracted with dichloromethane (5×10 mL). The combined organic fractions were dried over Na₂SO₄, concentrated, and chromatographed (hexane/EtOAc, 10:1). The product was obtained as a light brown solid (14.4 mg, 0.05 mmol) in a 24% yield: ¹H NMR (500 MHz, CDCl₃) δ 12.12 (s, 2H), 7.38 (d, J=2.2 Hz, 2H), 7.26 (dd, J=8.5, 2.2 Hz, 2H), 6.91 (d, J=8.5 Hz, 2H), 2.86 (s, 4H), 2.56 (s, 6H); ¹³C NMR (125 MHz, CDCl₃) δ 204.5, 160.9, 137.1, 131.5, 130.4, 119.5, 118.5, 37.2, 26.8; IR (neat) 3033, 2861, 1643, 1586, 1480, 1421, 1366, 1345, 1321, 1303, 1263, 1201, 1147, 1125, 1069, 1024, 960, 886, 845, 788, 635, 541, 472 cm⁻¹; HRMS (ESI-TOF) m/z 299.1283 calc for C₁₈H₁₉O₄ [M+H]⁺, found 299.1293.

### Example 58: 1,3-bis(3-methoxyphenyl)propane

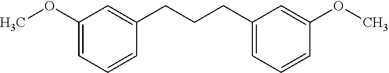

To a flame dried round bottom flask, was added Pd(OAc)₂ (19.67 mg, 0.09) and NaBH₄ (33.13 mg, 0.88 mmol). (E)-1,3-Bis(3-methoxyphenyl)prop-2-en-1-one (235 mg, 0.88 mmol) was dissolved in CHCl₃ (1 mL, 0.9 M) and the solution was added to the round bottom flask with the solids. With a balloon as an outlet for H₂ gas produced, MeOH (4 mL, 0.23 M) was slowly added to the flask. After reaction completion, the mixture was filtered through celite. The solvent was evaporated and the product was chromatographed (hexane/EtOAc, 10:1). The product was obtained as a dark orange liquid (16.7 mg, 0.07 mmol) in a 8% yield: ¹H NMR (500 MHz, CDCl₃) δ 7.20 (dd, J=8.2, 1.2 Hz, 2H), 6.79 (d, J=7.5, 2H) 6.75-6.73 (m, 4H), 3.80 (s, 6H), 2.64 (t, J=7.5, 4H), 1.96 (m, 2H); ¹³C NMR (125 MHz, acetone-D₆) δ 159.8, 143.8, 129.1, 120.5, 113.9, 111.0, 54.4, 35.2, 32.8. IR (neat) 2936, 2834, 1601, 1583, 1487, 1453, 1436, 1313, 1286, 1258, 1190, 1164, 1151, 1084, 1044, 996, 866, 775, 752, 737, 694, 571 cm⁻¹; FIRMS (EI-TOF) m/z=256.1463 calc for C₁₇H₂₀O₂ [M]⁺, found 256.1439.

### Example 59: General Procedure D Monoalkylation of Bibenzyl Analogs

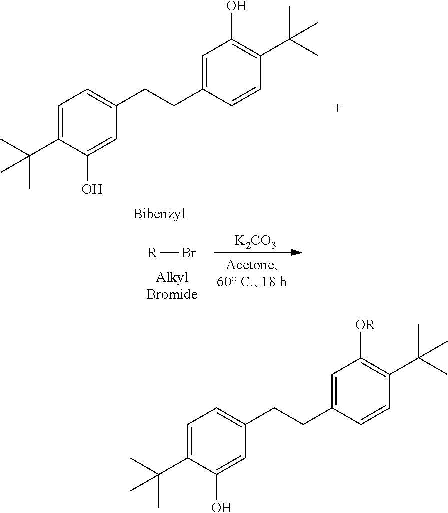

To a 10 mL microwave vial was added the bibenzyl analog (1 equiv), K₂CO₃ (1.5 equiv), and HPLC grade acetone (0.2 M). The vial was sealed with a septum and, under an inert atmosphere, the alkyl bromide (0.9 equiv) added. The septum was replaced with a crimping cap and the vessel was sealed and stirred for the indicated time at 60° C. When the reaction is finished, it is quenched with H₂O (15 mL). The resultant mixture is extracted with dichloromethane (5×15 mL) and ethyl acetate (5×15 mL), and then washed with brine (5×10 mL). The combined organic fractions were dried over Na₂SO₄, concentrated, and chromatographed using 10% ethyl acetate/hexane to afford the product.

### Example 60: (tert-butyl)-5-(4-(tert-butyl)-3-methoxyphenethyl)phenol (1H-2)

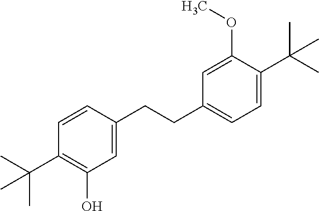 [SMILES: COc1cc(CCc2ccc(C(C)(C)C)c(O)c2)ccc1C(C)(C)C]

Following general procedure D, the product was obtained as a light yellow liquid (7.3 mg, 0.021 mmol) in a 40% yield: ¹H NMR (500 MHz, CDCl₃) δ 7.17 (d, J=8.0 Hz, 2H), 6.74 (d, J=8.0 Hz, 2H), 6.64 (s, 1H), 6.50 (s, 1H), 4.62 (s, 1H), 3.78 (s, 3H), 2.84 (m, 4H), 1.39 (s, 9H), 1.35 (s, 9H).

### Example 61: 2-(tert-butyl)-5-(4-(tert-butyl)-3-propoxyphenethyl)phenol

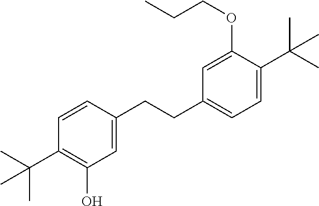 [SMILES: CCCOc1cc(CCc2ccc(C(C)(C)C)c(O)c2)ccc1C(C)(C)C]

Following general procedure D, the product was obtained as a light yellow liquid (13.4 mg, 0.036 mmol) in a 40% yield: ¹H NMR (500 MHz, CDCl₃) δ 7.20 (dd, J=7.8, 2.0 Hz, 2H), 6.75 (d, J=7.2 Hz, 1H), 6.74 (d, J=6.6 Hz, 1H), 6.65 (s, 1H), 6.52 (s, 1H), 4.66 (s, 1H), 3.90 (t, J=6.5 Hz, 2H), 2.84 (s, 4H), 1.86 (s, J=6.5 Hz, 2H), 1.40 (d, J=0.8 Hz, 9H), 1.38 (d, J=0.75 Hz, 9H), (td, J=6.5, 0.8 Hz, 3H).

### Example 62: 2-(tert-butyl)-5-(4-(tert-butyl)-3-(octyloxy)phenethyl)phenol

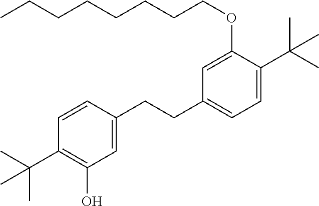 [SMILES: CCCCCCCCOc1cc(CCc2ccc(C(C)(C)C)c(O)c2)ccc1C(C)(C)C]

Following general procedure D, the product was obtained as a light yellow liquid (14.7 mg, 0.034 mmol) in a 49% yield: ¹H NMR (500 MHz, CDCl₃) δ 7.19 (dd, J=7.9, 1.8 Hz, 2H) 6.77-6.74 (m, 2H), 6.66 (s, 1H), 6.53 (s, 1H), 4.69 (s, 1H), 3.93 (t, J=6.5 Hz, 2H), 2.85-2.83 (m, 4H), 1.83 (p, J=7 Hz, 2H), 1.51 (p, J=7.5, 3H), 1.41 (s, 9H), 1.39 (s, 9H), 1.34-1.26 (m, 10H).

### Example 63: 2-(tert-butyl)-5-(4-(tert-butyl)-3-(prop-2-yn-1-yloxy)phenethyl)phenol

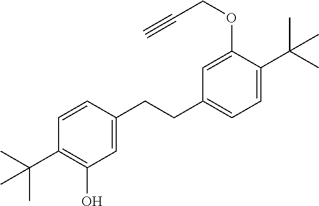 [SMILES: C#CCOc1cc(CCc2ccc(C(C)(C)C)c(O)c2)ccc1C(C)(C)C]

Following general procedure D, the product was obtained as a light yellow liquid (12.7 mg, 0.035 mmol) in a 45% yield: ¹H NMR (500 MHz, CDCl₃) δ 7.21 (dd, J=7.9, 3.5 Hz, 2H), 6.80 (d, J=7.9 Hz, 1H), 6.76-6.75 (m, 2H), 6.53 (s, 1H), 4.72 (s, 1H), 4.68 (d, J=2.2 Hz, 2H), 2.90-2.82 (m, 4H), 2.50 (t, J=2.2 Hz, 1H), 1.41 (s, 9H), 1.39 (s, 9H).

### Example 64: 5,5′-(ethane-1,2-diyl)bis(2-ethylphenol)

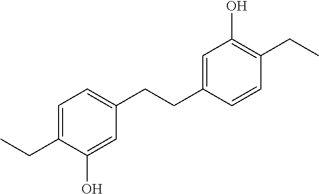 [SMILES: CCc1ccc(CCc2ccc(CC)c(O)c2)cc1O]

In a microwave vial 5,5′-(ethane-1,2-diyl)bis(2-(1-hydroxyethyl)phenol) (14.5 mg, 0.048 mmol) was dissolved in chlorobenzene (0.1 M). The solution was cooled to 0° C., and then BH₃.SMe₂ was slowly added dropwise. After stirring at room temperature for 15 min, the temperature was raised to 80° C. for 3 h and then to 130° C. for 18 h. The reaction was quenched with Na₂CO₃ (10 mL). The resultant mixture was extracted with dichloromethane (5×10 mL) and washed with brine (5×10 mL). The combined organic fractions were dried over Na₂SO₄, concentrated, and chromatographed (hexane/EtOAc, 10:1). The product was obtained as a white solid (9.9 mg, 0.037 mmol) in a 74% yield: ¹H NMR (500 MHz, CDCl₃) δ 7.04 (d, J=7.6 Hz, 2H), 6.72 (d, J=7.6 Hz, 2H), 6.59 (s, 2H), 4.63 (s, 2H), 2.80 (s, 4H), 2.59 (q, J=7.5 Hz, 4H), 1.22 (t, J=7.5 Hz, 6H).

### Example 65: di-tert-butyl ((((ethane-1,2-diylbis(2-hydroxy-4,1-phenylene))bis(methylene))bis(azanediyl))bis(ethane-2,1-diyl))dicarbamate

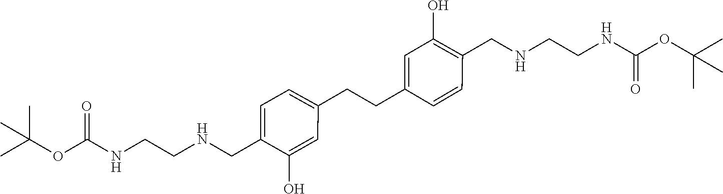 [SMILES: CC(C)(C)OC(=O)NCCNCc1ccc(CCc2ccc(CNCCNC(=O)OC(C)(C)C)c(O)c2)cc1O]

In a microwave vial was added 4,4′-(ethane-1,2-diyl)bis(2-hydroxybenzaldehyde) (30 mg, 0.111 mmol), tert-butyl (2-aminoethyl)carbamate (35.2 μL, 0.222 mmol), and HPLC grade MeOH (2.22 mL, 0.05 M). The vial was sealed with a crimp cap and stirred at 70° C. for 2 h. The vial was opened to air, and then NaBH₄ (25.2 mg, 0.666 mmol) was slowly added. After stirring 18 h at rt, the reaction was quenched with H₂O (20 mL). The resultant mixture was extracted with dichloromethane (5×10 mL) and ethyl acetate (5×10 mL) and washed with brine (5×10 mL). The combined organic fractions were dried over Na₂SO₄, and concentrated to afford the product as a dark brown gel (61.1 mg, 0.109 mmol) in a 98% yield: ¹H NMR (500 MHz, CDCl₃) δ 6.88 (s, 2H), 6.67 (s, 2H), 6.62 (s, 2H), 4.79 (s, 2H) 3.97 (s, 4H), 3.28 (s, 4H), 2.81 (s, 4H), 2.78 (s, 4H), 1.43 (s, 18H); ¹³C NMR (125 MHz, CDCl₃) δ 157.9, 156.2, 142.9, 128.3, 119.9, 119.2, 116.3, 53.5, 52.1, 48.4, 40.1, 37.5, 28.4.

### Example 66: 5,5′-(ethane-1,2-diyl)bis(2-0(2-aminoethyl)amino)methyl)phenol)

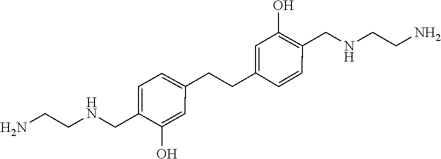 [SMILES: NCCNCc1ccc(CCc2ccc(CNCCN)c(O)c2)cc1O]

In a microwave vial was slowly added trifluoroacetic acid (132.5 μL, 1.73 mmol) to a solution of di-tert-butyl ((((ethane-1,2-diylbis(2-hydroxy-4,1-phenylene))bis(methylene))bis(azanediyl))bis(ethane-2,1-diyl))dicarbamate (49.6 mg, 0.087 mmol) in anhydrous dichloromethane (0.9 mL, 0.1 M). The mixture was stirred for 24 h and then concentrated to afford the product as a light brown solid (15.1 mg, 0.042 mmol) in a 96% yield: ¹H NMR (500 MHz, MeOD) δ 7.21 (d, J=7.4 Hz, 2H), 6.77 (s, 2H), 6.73 (d, J=7.4 Hz, 2H), 4.24 (s, 4H), 3.34-3.31 (m, 8H), 2.84 (s, 4H); ¹³C NMR (125 MHz, MeOD) δ 156.2, 145.5, 131.2, 119.9, 115.0, 114.5, 46.9, 43.5, 37.0, 35.3.

### Example 67: 1-(but-3-en-1-yl)-3-methoxybenzene

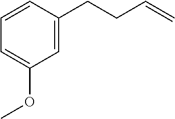

To a solution of 1-(bromomethyl)-3-methoxybenzene (1 mmol) in anhydrous THF (0.2 M) was slowly added a solution of allylmagnesium bromide (1 M in diethyl ether) (4.5 mmol) at 0° C. The reaction was stirred for 2-3 h at room temperature. The reaction was cooled to 0° C. and quenched via drop-wise addition of sat. NH₄Cl (5 mL). After stirring at room temperature for 10 minutes, the product was extracted with diethyl ether (5×20 mL). The combined organic fractions were dried over Na₂SO₄, concentrated, and chromatographed using 10% ethyl acetate/hexane to afford the product.

### Example 68: (E)-3,3′-(but-1-ene-1,4-diyl)bis(methoxybenzene)

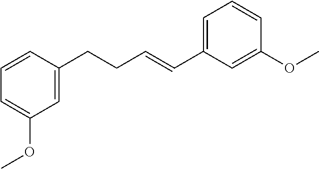 [SMILES: COc1cccc(/C=C/CCc2cccc(OC)c2)c1]

To a microwave vial was added Pd₂(dba)₃ (0.02 mmol) and [HP(tert-Bu)₃]BF₄ (0.06 mmol). Under a flow of argon, DMSO (1.1 mL) and DMF (1.1 mL) were added to 1-(but-3-en-1-yl)-3-methoxybenzene (1.5 mmol). That solution, NEt₃ (1.5 mmol), and 3-bromoanisole (1.0 mmol) were added to the microwave vial. The reaction vial was sealed with a crimping cap and heated to 100° C. for 18 h. The reaction mixture was poured into 100 mL of sat. NaHCO₃ and extracted with toluene (5×20 mL). The combined organic fractions were dried over Na₂SO₄, concentrated, and recrystallized in hexanes and ethyl acetate to afford the product.

### Example 69: 1,4-bis(3-methoxyphenyl)butane

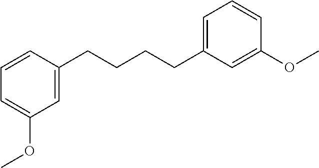 [SMILES: COc1cccc(CCCCc2cccc(OC)c2)c1]

To a round bottom flask with (E)-3,3′-(but-1-ene-1,4-diyl)bis(methoxybenzene (1 equiv.) and palladium, 10% on carbon (0.9 equiv.) was added anhydrous ethanol (0.1 M) Hydrogen was added via active purge and allowed to stir for 18 h. After completion of the reaction, the flask was purged with argon and filtered through a bed of celite. The solvent was evaporated and product was chromatographed using 10% ethyl acetate/hexane to afford the product.

### Example 70: 3,3′-(butane-1,4-diyl)diphenol

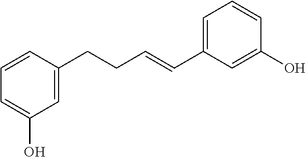 [SMILES: Oc1cccc(/C=C/CCc2cccc(O)c2)c1]

To a solution of 1,4-bis(3-methoxyphenyl)butane (0.028 mmol) in anhydrous dichloromethane (0.2 M) at −78° C. was added BBr₃ (1 M solution in dichloromethane) (6.40 mmol) drop-wise under argon flow. The reaction stirred for 30 min, then slowly raised to room temperature and stirred for 12 h. The reaction was quenched via drop wise addition of H₂O, and extracted using ethyl acetate (5×30 mL). The combined organic fractions were dried over Na₂SO₄, concentrated, and chromatographed (hexane/EtOAc, 10:1) to afford the product.

### Example 71: 5,5′-(butane-1,4-diyl)bis(2-(tert-butyl)phenol)

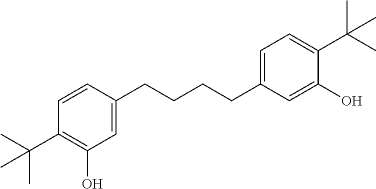 [SMILES: CC(C)(C)c1ccc(CCCCc2ccc(C(C)(C)C)c(O)c2)cc1O]

To a solution of the 3,3′-(butane-1,4-diyl)diphenol. (1 equiv.) in dichloromethane (0.2 M) at 0° C. was added tert-butyl alcohol (2.2 equiv) and H₂SO₄ (2 equiv.). The reaction was stirred for 18 h and monitored via TLC. When the reaction is finished, it is quenched with a saturated solution of NaHCO₃(10 mL), extracted with dichloromethane (5×15 mL) and ethyl acetate (5×15 mL), and washed with brine (5×10 mL). The combined organic fractions were dried over Na₂SO₄, concentrated, and chromatographed using 10% ethyl acetate/hexane to afford the product.

### Example 72: 1-methoxy-3-(pent-4-en-1-yl)benzene

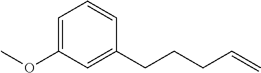

To a solution of 1-(bromomethyl)-3-methoxybenzene (1 mmol) in anhydrous THF (0.2 M) was slowly added a solution of but-3-en-1-ylmagnesium bromide (1 M in diethyl ether) (4.5 mmol) at 0° C. The reaction was stirred for 2-3 h at room temperature. The reaction was cooled to 0° C. and quenched via drop-wise addition of sat. NH₄Cl (5 mL). After stirring at room temperature for 10 minutes, the product was extracted with diethyl ether (5×20 mL). The combined organic fractions were dried over Na₂SO₄, concentrated, and chromatographed using 10% ethyl acetate/hexane to afford the product.

### Example 73: (E)-3,3′-(pent-1-ene-1,5-diyl)bis(methoxybenzene)

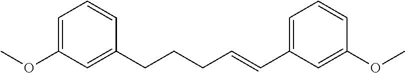

To a microwave vial was added Pd₂(dba)₃ (0.02 mmol) and [HP(tert-Bu)₃]BF₄ (0.06 mmol). Under a flow of argon, DMSO (1.1 mL) and DMF (1.1 mL) were added to 1-methoxy-3-(pent-4-en-1-yl)benzene (1.5 mmol). That solution, NEt₃ (1.5 mmol), and 3-bromoanisole (1.0 mmol) were added to the microwave vial. The reaction vial was sealed with a crimping cap and heated to 100° C. for 18 h. The reaction mixture was poured into 100 mL of sat. NaHCO₃ and extracted with toluene (5×20 mL). The combined organic fractions were dried over Na₂SO₄, concentrated, and recrystallized in hexanes and ethyl acetate to afford the product.

### Example 74: 1,5-bis(3-methoxyphenyl)pentane

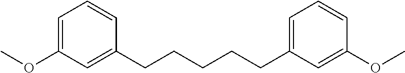

To a round bottom flask with (E)-3,3′-(pent-1-ene-1,5-diyl)bis(methoxybenzene) (1 equiv.) and palladium, 10% on carbon (0.9 equiv.) was added anhydrous ethanol (0.1 M) Hydrogen was added via active purge and allowed to stir for 18 h. After completion of the reaction, the flask was purged with argon and filtered through a bed of celite. The solvent was evaporated and product was chromatographed using 10% ethyl acetate/hexane to afford the product.

### Example 75: 3,3′-(pentane-1,5-diyl)diphenol

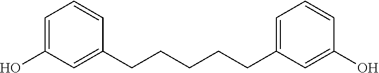 [SMILES: Oc1cccc(CCCCCc2cccc(O)c2)c1]

To a solution of 1,5-bis(3-methoxyphenyl)pentane (0.028 mmol) in anhydrous dichloromethane (0.2 M) at −78° C. was added BBr₃ (1 M solution in dichloromethane) (6.40 mmol) drop-wise under argon flow. The reaction stirred for 30 min, then slowly raised to room temperature and stirred for 12 h. The reaction was quenched via drop wise addition of H₂O, and extracted using ethyl acetate (5×30 mL). The combined organic fractions were dried over Na₂SO₄, concentrated, and chromatographed (hexane/EtOAc, 10:1) to afford the product.

### Example 76: 5,5′-(pentane-1,5-diyl)bis(2-(tert-butyl)phenol)

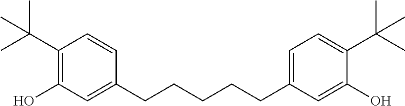 [SMILES: CC(C)(C)c1ccc(CCCCCc2ccc(C(C)(C)C)c(O)c2)cc1O]

To a solution of 3,3′-(pentane-1,5-diyl)diphenol (1 equiv.) in dichloromethane (0.2 M) at 0° C. was added tert-butyl alcohol (2.2 equiv) and H₂SO₄ (2 equiv.). The reaction was stirred for 18 h and monitored via TLC. When the reaction is finished, it is quenched with a saturated solution of NaHCO₃(10 mL), extracted with dichloromethane (5×15 mL) and ethyl acetate (5×15 mL), and washed with brine (5×10 mL). The combined organic fractions were dried over Na₂SO₄, concentrated, and chromatographed using 10% ethyl acetate/hexane to afford the product.

### Example 77: Materials

Streptococcus mutans wild-type strain UA159 was provided by Dr. Bettina Buttaro from Temple University Medical School, Philadelphia, Pa. Streptococcus gordonii strain DL1 and Streptococcus sanguinis strain 10904 were provided by Dr. Robert G. Quivey from University of Rochester Medical School. Streptococcus gordonii strain DL1 may be obtained from the American Type Culture Collection under accession #49818. Streptococcus sanguinis strain 10904 may be obtained from the National Collection of Type Cultures under accession #10904. Bacteria were routinely maintained on in Bacto™ Todd-Hewitt (TH) agar plates and liquid cultures were grown in in Bacto™ Todd-Hewitt broth (THB). For growth of biofilms, THB was supplemented with 0.1% sucrose. Incubation was stagnant at 37° C. Bacterial strains Methicillin-Resistant Staphylococcus aureus HPV107, Staphylococcus epidermidis Fussel [NCTC 11047, R. Hugh 2466], Streptococcus sobrinus SL1 [CCM 6070, CNCTC 9/89], Enterococcus faecalis NCTC 775 [DSM 20478, JCM 8726, NCDO 581, Tissier], Pseudomonas aeruginosa [CCEB 481, MDB strain BU 277, NCIB 8295, NCPPB 1965, NCTC 10332, NRRL B-771, R. Hugh 815], Serratia marcescens BS 303 [CDC 813-60, NCIB 9155, NCTC 10211], and Acinetobacter baumannii 2208 [81, DSM 6974] were purchased from the American Type Culture Collection.

### Example 78: Bacterial Assays

Stock solutions of compounds (10 mM) were serial diluted (200 μL, 100 μL, 50 μL, 10 μL, and 2 μL) in THB media in flat-bottom 96-well microtiter plates (total volume 100 μL). Bacterial cultures were grown in brain heart infusion or nutrient broth media to mid-exponential phase, back diluted to an OD of 0.1 and then inoculated into the 96-well plate to reach a final volume of 200 μL. Plates were inoculated at 37° C. in 5% CO₂ for 20-24 hours upon which time wells were evaluated visually for bacterial growth and/or the wells' OD₆₃₀ was evaluated via spectrophotometer. The MIC was determined as the lowest concentration of compound resulting in no bacterial growth visible to the naked eye and the difference of measured and background OD₆₃₀ is less than 0.1. DMSO controls corresponding to each test concentration were performed. Biological triplicates were performed to confirm results.

A. S. mutans Biofilm Model

Stock solutions of the compounds (10 mM) were serial diluted in THB media supplemented with 0.1% sucrose (w/v) in flat-bottom 96-well microtiter plates (total volume 100 μL). Bacterial cultures were grown to mid-exponential phase, back diluted to an OD of 0.1 and then inoculated into the 96-well plate to reach a final volume of 200 μL. Plates were incubated at 37° C. in 5% CO₂ for 24 hours (early stage biofilm) upon which time wells were evaluated visually for bacterial growth. DMSO controls corresponding to each test concentration were performed. Biological triplicates were performed to confirm results.

B. Crystal Violet (Biofilm Mass)

Biofilm assay was performed (above) and wells are washed with 200 μL of DI H₂O and dried for 24 hours at 37° C. Dried plates were incubated for 10 minutes at room temperature with 50 μL of 1% w/v crystal violet (25% ethanol in H₂O). Excess crystal violet was removed via aspiration. 200 μL of DI H₂O was added to each well and aspirated (repeat until all excess crystal violet is removed). Plates were then inverted and dried at 37° C. for 3-5 hours. Crystal violet stain biofilm mass was dissolved with 200 μL of 95% ethanol, 100 μL of which was then transferred to a fresh flat-bottom 96-well plate for absorbance measurements at 595 nm. DMSO controls corresponding to each test concentration were performed. Biological triplicate was performed to confirm results. MBIC₅₀ value corresponds to the concentration that inhibits the formation of biofilm by 50%.

C. S. mutans MBC Assay

MIC assay was performed (above) and each well diluted (log-dilution) into a new 96-well microtiter plate. Five μL from each dilution was then plated on THB agar plates and incubated for 24 hours. Colony counts were performed to determine MBC which is defined as the concentration which there is a 3-log reduction in CFU count which corresponds to 99.9% bacterial death.

D. Results

The bioactivity of the bis-phenol compounds was analyzed against a panel of representative oral bacteria via MIC and MBC assays (Table 4). Out of the twenty-six compounds, four compounds showed significant inhibition at low concentrations (≤16 μM). Compounds C2, B5, B8 and B11 were the most impressive with MIC values of 2 μM, 8 μM, 16 μM and 16 μM respectively against planktonic S. mutans. The compounds were also tested against two commensal strains that are early colonizers: S. gordonii and S. sanguinis. Generally, the MICs for these commensal strains mirrored the values for the pathogenic S. mutans hinting at a broad-spectrum inhibitory mechanism.

Minimum bactericidal concentration (MBC) assays were undertaken. MIC assays were initially performed in a 5% CO₂-supplemented environment to promote growth of S. mutans in an environment that most closely mimics a healthy oral cavity. See, Table 2 which provides a summary of MIC (S. mutans, S. sanguinis, and S. gordonii) and MBC (S. mutans) values for the compounds. MIC and MBC values were completed in biological triplicate. MBC assays were completed for compounds with MIC values <32 μM.

The MIC of honokiol was determined to be 250 μM (66.6 μg/mL), which was in stark contrast to the literature value of 10 μg/mL (Table 4). After revisiting the original procedures, it was noted that the original assays were completed in an aerobic environment, which precluded the growth of S. mutans. When aerobic conditions were employed in the assay, the potency of honokiol increased to 125 μM (33.3 μg/mL). These results demonstrate that although S. mutans growth was inhibited by honokiol, the overall efficacy of the compound will be less under physiological conditions. Furthermore, honokiol is unable to inhibit biofilm growth and was not bactericidal at concentrations of 250 μM or lower.

TABLE 4

| | | MIC | MIC | MIC | MBC |
|---|---|---|---|---|---|
| Analog | | S. mutans | S. sanguinis | S. gordonii | S. mutans |
| A | 1 | 250 | 125 | 125 | — |
| | 2 | 32 | 32 | 32 | 63 |
| | 3 | >250 | >250 | >250 | — |
| | 4 | >250 | >250 | >250 | — |
| | 5 | >250 | >250 | >250 | — |
| | 6 | >250 | >250 | >250 | — |
| | 7 | 32 | 32 | 16 | 32 |
| | 8 | >250 | 125 | 125 | — |
| | 9 | >250 | >250 | >250 | — |
| | 10 | >250 | 250 | 250 | — |
| | 11 | >250 | >250 | >250 | — |
| B | 1 | >250 | >250 | >250 | — |
| | 2 | >250 | >250 | >250 | — |
| | 3 | 63 | 63 | 63 | — |
| | 4 | 125 | 125 | 125 | — |
| | 5 | 8 | 4 | 16 | 125 |
| | 6 | >250 | >250 | >250 | — |
| | 7 | 125 | 63 | 125 | — |
| | 8 | 16 | 8 | 16 | 32 |
| | 9 | 32 | 32 | 32 | 32 |
| | 10 | 32 | 8 | 8 | 32 |
| | 11 | 16 | 8 | 16 | 63 |
| | 12 | >250 | 250 | 250 | — |
| | 13 | >250 | >250 | >250 | — |
| | 14 | 63 | 16 | 32 | — |
| C | 1 | >250 | 125 | 125 | — |
| | 2 | 2 | 2.5 | 1.25 | 4 |

The compounds were then tested for their ability to inhibit the formation of S. mutans biofilm. All of the compounds potently deterred the formation of biofilms when the cells were grown in the presence of sucrose, albeit at the previously determined MIC values. This may be due to the fact that the compounds are targeting the bacteria in a general fashion and do not show any preferential killing to biofilms. The compounds were further tested for their bacteriostatic or bactericidal properties. A regrow analysis was completed to determine the MBC values of the active compounds against S. mutans. The MBC values reported refer to the concentration at which there is a 3-Log reduction in CFU count corresponding to 99.9% bacterial cell death. Compound C₂ had the lowest MBC value at 4 μM confirming that the molecule is bactericidal (Macia, Clin. Microbiol. Infect. 2014, 20, 981-990). Compounds B8 and B11 were also shown to be bactericidal; however, the MIC and MBC of compound B5 differs by four dilutions hinting at a bacteriostatic mechanism. These findings suggest that compounds C2 and B5 may be inhibiting the growth of S. mutans by different mechanisms.

### Example 79: Additional Biological Studies

Compounds described herein were tested against the bacteria described in Example 28 and further against gram positive methicillin-resistant Staphylococcus aeures (MRSA), and Streptococcus sobrinus (Table 5) and the gram negative Acinetobacter baumannii, Pseudomonas aeruginosa, and Serratia marcescens (Table 6).

TABLE 5
Gram Positive Bacteria

| Compound | S. epidermidis | E. faecalis | MRSA | S. sobrinus | S. mutans |
|---|---|---|---|---|---|
| 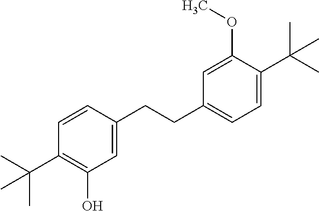 [SMILES: COc1cc(CCc2ccc(C(C)(C)C)c(O)c2)ccc1C(C)(C)C] | 10 | >200 | 4 | 6 | 8 |
| 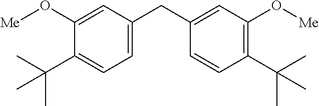 [SMILES: COc1cc(Cc2ccc(C(C)(C)C)c(OC)c2)ccc1C(C)(C)C] | >200 | >200 | NT | NT | NT |
| 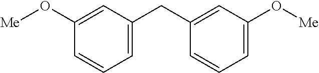 [SMILES: COc1cccc(Cc2cccc(OC)c2)c1] | >200 | >200 | NT | NT | NT |
| 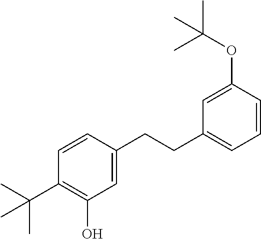 [SMILES: CC(C)(C)Oc1cccc(CCc2ccc(C(C)(C)C)c(O)c2)c1] | >200 | >200 | NT | NT | N/A |
| 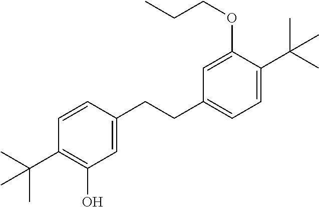 [SMILES: CCCOc1cc(CCc2ccc(C(C)(C)C)c(O)c2)ccc1C(C)(C)C] | 50 | NT | 50 | NT | NT |
| 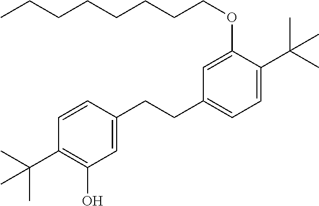 [SMILES: CCCCCCCCOc1cc(CCc2ccc(C(C)(C)C)c(O)c2)ccc1C(C)(C)C] | >100 | >100 | >100 | NT | NT |
| 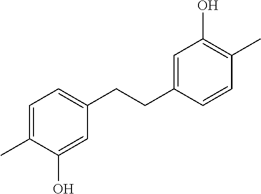 | >200 | >200 | NT | NT | NT |
| 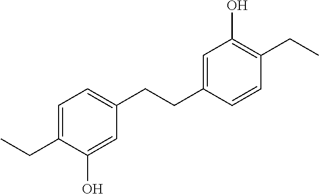 [SMILES: CCc1ccc(CCc2ccc(CC)c(O)c2)cc1O] | 100 | 100 | NT | NT | NT |
| 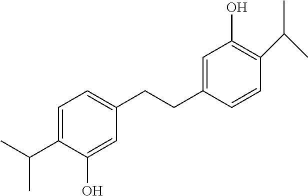 [SMILES: CC(C)c1ccc(CCc2ccc(C(C)C)c(O)c2)cc1O] | 50 | 50 | NT | NT | NT |
| 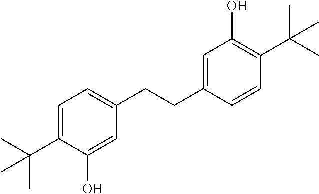 [SMILES: CC(C)(C)c1ccc(CCc2ccc(C(C)(C)C)c(O)c2)cc1O] | 10 | 10 | 6 | 6 | 2 |
| 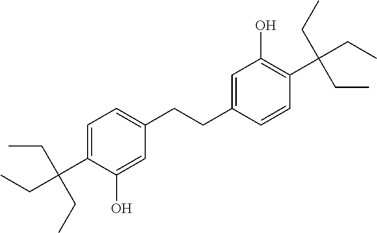 [SMILES: CCC(CC)(CC)c1ccc(CCc2ccc(C(CC)(CC)CC)c(O)c2)cc1O] | 10 | >200 | 11 | NT | NT |
| 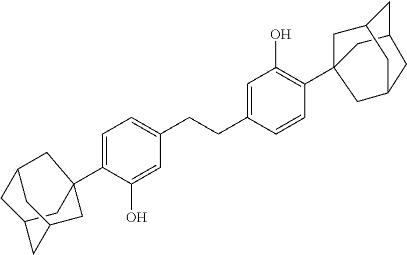 [SMILES: Oc1cc(CCc2ccc(C34CC5CC(CC(C5)C3)C4)c(O)c2)ccc1C12CC3CC(CC(C3)C1)C2] | >200 | >200 | NT | NT | NT |
| 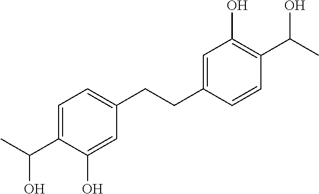 [SMILES: CC(O)c1ccc(CCc2ccc(C(C)O)c(O)c2)cc1O] | >200 | >200 | NT | NT | NT |
| 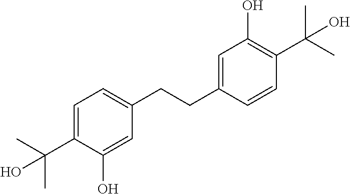 [SMILES: CC(C)(O)c1ccc(CCc2ccc(C(C)(C)O)c(O)c2)cc1O] | >200 | >200 | NT | NT | N/A |
| 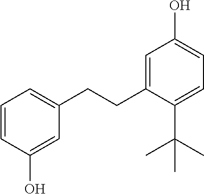 [SMILES: CC(C)(C)c1ccc(O)cc1CCc1cccc(O)c1] | NT | NT | NT | NT | NT |
| 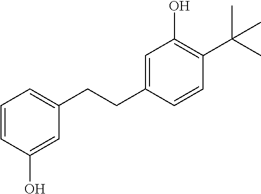 [SMILES: CC(C)(C)c1ccc(CCc2cccc(O)c2)cc1O] | 50 | >200 | 25 | 25 | 16 |
| 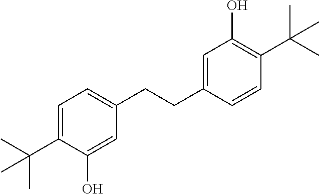 [SMILES: CC(C)(C)c1ccc(CCc2ccc(C(C)(C)C)c(O)c2)cc1O] | 10 | 10 | 6 | 6 | 2 |
| 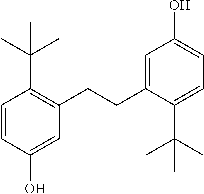 [SMILES: CC(C)(C)c1ccc(O)cc1CCc1cc(O)ccc1C(C)(C)C] | 50 | >200 | NT | 9 | NT |
| 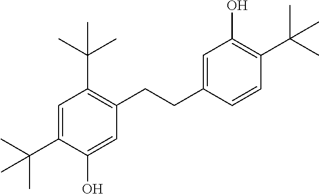 [SMILES: CC(C)(C)c1ccc(CCc2cc(O)c(C(C)(C)C)cc2C(C)(C)C)cc1O] | 25 | >200 | NT | NT | NT |
| 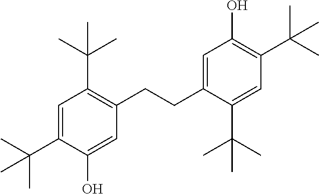 [SMILES: CC(C)(C)c1cc(C(C)(C)C)c(CCc2cc(O)c(C(C)(C)C)cc2C(C)(C)C)cc1O] | >200 | >200 | NT | NT | NT |
| 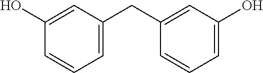 [SMILES: Oc1cccc(Cc2cccc(O)c2)c1] | >200 | >200 | NT | NT | NT |
| 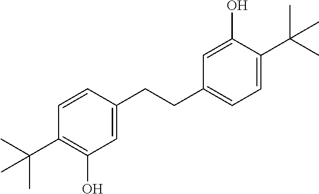 [SMILES: CC(C)(C)c1ccc(CCc2ccc(C(C)(C)C)c(O)c2)cc1O] | 10 | >200 | NT | NT | NT |
| 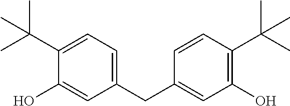 [SMILES: CC(C)(C)c1ccc(Cc2ccc(C(C)(C)C)c(O)c2)cc1O] | 50 | 50 | 9 | NT | 2 |
| 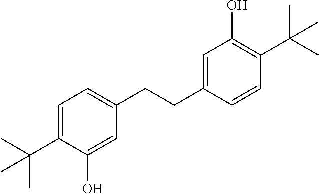 [SMILES: CC(C)(C)c1ccc(CCc2ccc(C(C)(C)C)c(O)c2)cc1O] | 10 | 10 | 6 | 6 | 2 |
| 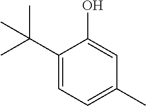 | >200 | >200 | >200 | NT | NT |
| 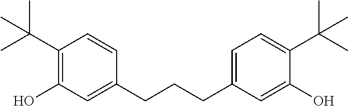 [SMILES: CC(C)(C)c1ccc(CCCc2ccc(C(C)(C)C)c(O)c2)cc1O] | 10 | >200 | 11 | NT | NT |
| 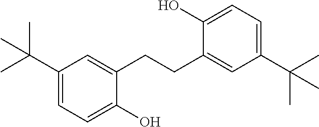 [SMILES: CC(C)(C)c1ccc(O)c(CCc2cc(C(C)(C)C)ccc2O)c1] | 10 | >200 | NT | NT | NT |
| 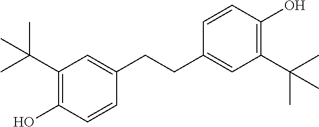 [SMILES: CC(C)(C)c1cc(CCc2ccc(O)c(C(C)(C)C)c2)ccc1O] | 10 | >100 | 11 | NT | NT |
| 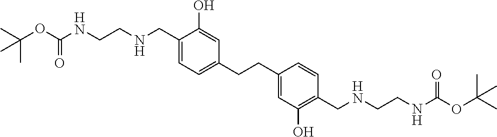 [SMILES: CC(C)(C)OC(=O)NCCNCc1ccc(CCc2ccc(CNCCNC(=O)OC(C)(C)C)c(O)c2)cc1O] | 200 | NT | NT | NT | NT |
| 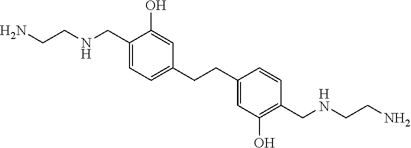 [SMILES: NCCNCc1ccc(CCc2ccc(CNCCN)c(O)c2)cc1O] | >200 | NT | NT | NT | NT |
| 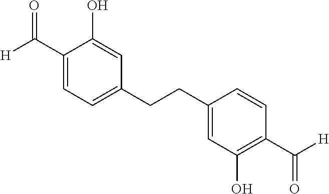 [SMILES: O=Cc1ccc(CCc2ccc(C=O)c(O)c2)cc1O] | >200 | >200 | NT | NT | NT |
| 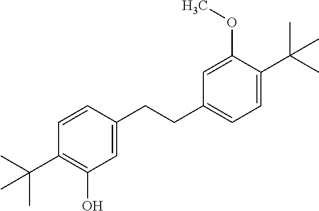 [SMILES: COc1cc(CCc2ccc(C(C)(C)C)c(O)c2)ccc1C(C)(C)C] | NT | NT | NT | NT | NT |
| 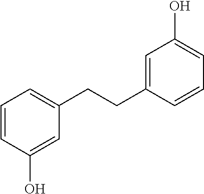 [SMILES: Oc1cccc(CCc2cccc(O)c2)c1] | NT | NT | NT | NT | NT |
| 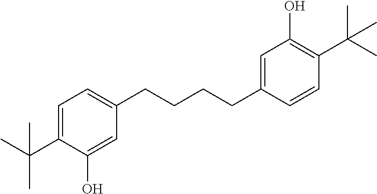 [SMILES: CC(C)(C)c1ccc(CCCCc2ccc(C(C)(C)C)c(O)c2)cc1O] | NT | NT | NT | NT | need to make |
| 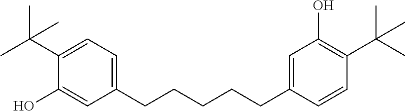 [SMILES: CC(C)(C)c1ccc(CCCCCc2ccc(C(C)(C)C)c(O)c2)cc1O] | NT | NT | NT | NT | need to make |

*NT = not tested

The following compound, as tested in the assay above, was impure. It is expected that this compound would have improved activity in a purer form. It would also be expected that compounds having smaller cycloalkyl groups in place of the adamantyl groups would have improved activity.

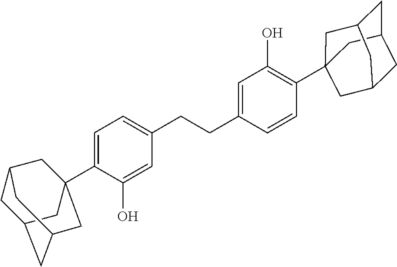 [SMILES: Oc1cc(CCc2ccc(C34CC5CC(CC(C5)C3)C4)c(O)c2)ccc1C12CC3CC(CC(C3)C1)C2]

TABLE 6
Gram Negative Bacteria

| Compound | A. baumannii | P. aeruginosa | S. marcescens |
|---|---|---|---|
| 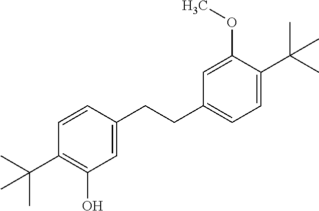 [SMILES: COc1cc(CCc2ccc(C(C)(C)C)c(O)c2)ccc1C(C)(C)C] | >200 | >200 | >200 |
| 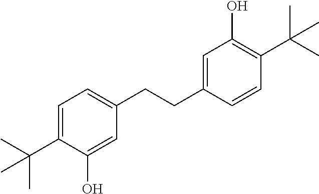 [SMILES: CC(C)(C)c1ccc(CCc2ccc(C(C)(C)C)c(O)c2)cc1O] | >200 | >200 | >200 |
| 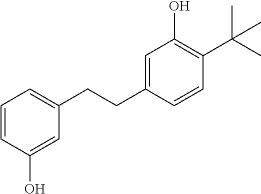 [SMILES: CC(C)(C)c1ccc(CCc2cccc(O)c2)cc1O] | >200 | >200 | >200 |
| 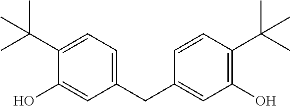 [SMILES: CC(C)(C)c1ccc(Cc2ccc(C(C)(C)C)c(O)c2)cc1O] | >200 | >200 | >200 |
| 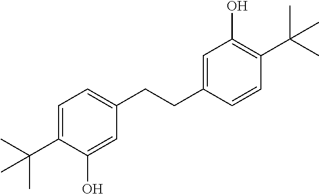 [SMILES: CC(C)(C)c1ccc(CCc2ccc(C(C)(C)C)c(O)c2)cc1O] | >200 | >200 | >200 |
| 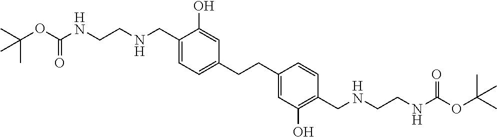 [SMILES: CC(C)(C)OC(=O)NCCNCc1ccc(CCc2ccc(CNCCNC(=O)OC(C)(C)C)c(O)c2)cc1O] | 200 | NT | NT |
| 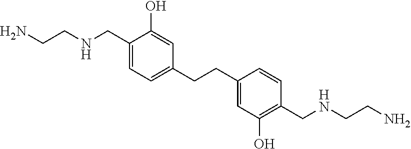 [SMILES: NCCNCc1ccc(CCc2ccc(CNCCN)c(O)c2)cc1O] | >200 | NT | NT |

*NT = not tested

These data show that the compounds described herein are active against a wide variety of bacteria.

### Example 80: Cell Lysis

The effects of the compounds described herein to lyse cells was evaluated in this example. Hemolysis assays were performed on mechanically defibrinated sheep blood (Hemostat Labs: DSB030). 1.5 mL of blood was placed into a microcentrifuge tube and centrifuged at 10,000 rpm for ten minutes. The supernatant was removed and then the cells were resuspended with 1 mL of phosphate-buffered saline (PBS). The suspension was centrifuged as previously, the supernatant was removed, and cells were resuspended two more times. The final cell suspension was diluted twentyfold with PBS. The twentyfold suspension dilution was then aliquoted into microcentrifuge tubes containing compound serially diluted in PBS. TritonX (1% by volume) served as a positive control (100% lysis marker) and sterile PBS served as a negative control (0% lysis marker). Samples were then placed in an incubator at 37° C. and shaken at 200 rpm. After 1 hour, the samples were centrifuged at 10,000 rpm for ten minutes. The absorbance of the supernatant was measured with a UV spectrometer at a 540 nm wavelength. See, e.g., Peng, Chem. Comm. 2011, 47, 4896-4898.

These data shows that compounds 1H and 3N perturb the membrane at 32 μM which is 4 dilutions higher than its MIC of 2 μM. See, Table 7 where values refer to concentration needed to lyse 20% of the red blood cells.

TABLE 7

| | Concentration (μM) | | | | | | | | | | | |
|---|---|---|---|---|---|---|---|---|---|---|---|---|
| Compound | 500 | 250 | 125 | 63 | 32 | 16 | 8 | 4 | 2 | 1 | 0.5 | 0.25 |
| 1H | 0.48 | 0.70 | 0.43 | 0.29 | 0.07 | −0.03 | −0.03 | −0.01 | −0.02 | −0.02 | 0.01 | 0.02 |
| 3N | 0.74 | 0.34 | 0.43 | 0.38 | 0.02 | −0.02 | −0.03 | 0.11 | −0.02 | −0.02 | −0.02 | 0.13 |
| 6* | 0.27 | 0.02 | −0.02 | 0.07 | −0.02 | −0.03 | −0.03 | 0.35 | −0.03 | −0.03 | −0.02 | 0.03 |
| 1H-2 | 0.33 | 0.21 | 0.12 | 0.02 | −0.02 | −0.03 | −0.03 | 0.16 | −0.03 | −0.02 | −0.02 | 0.13 |
| 1H-3 | 0.81 | 0.69 | 0.46 | 0.00 | −0.03 | −0.03 | 0.29 | −0.03 | −0.03 | −0.03 | 0.00 | 0.11 |
| 2E | 0.22 | 0.31 | −0.02 | −0.02 | −0.02 | −0.03 | 0.05 | 0.07 | −0.02 | −0.02 | −0.02 | 0.14 |
| QAC | 0.63 | 0.73 | 0.48 | 0.61 | 0.50 | 0.28 | 0.01 | 0.00 | 0.00 | 0.00 | −0.01 | 0.07 |
| DMSO | 0.21 | 0.04 | −0.02 | 0.08 | −0.02 | 0.13 | −0.03 | −0.02 | −0.02 | −0.02 | 0.00 | 0.26 |

*(Compound 6)

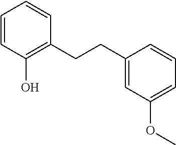 [SMILES: COc1cccc(CCc2ccccc2O)c1]

These data shows that compound 1H described herein lyses blood cells at high concentrations.

### Example 81: Cell Permeation Studies

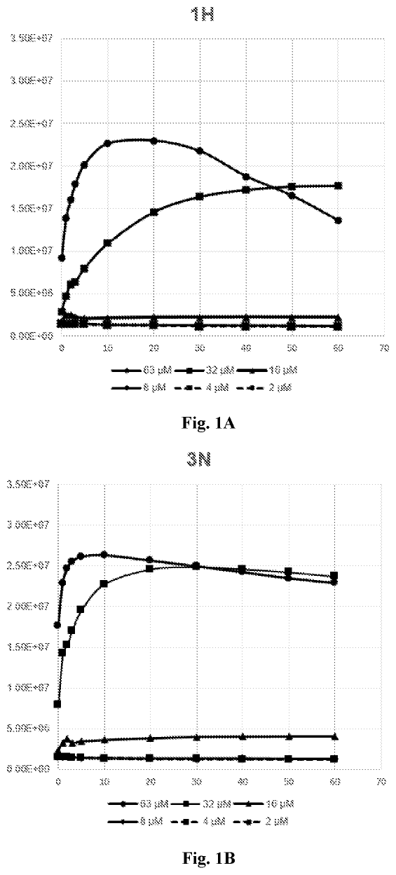

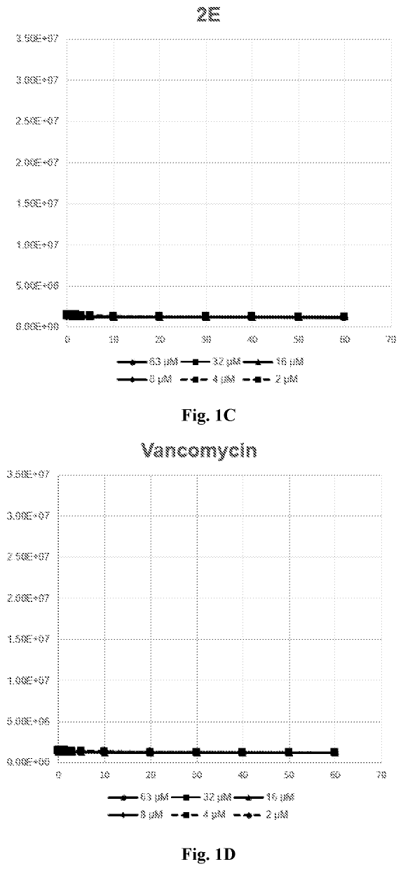

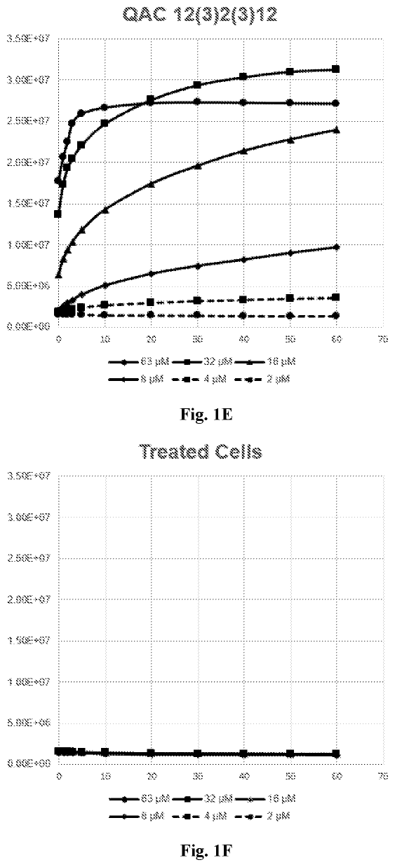

This example was performed to measure cell viability (i.e., cell permeabilization) by using a molecule that fluoresces when bound to DNA. The assay performed was a SYTOX™ assay green nucleic acid stain using compounds 1H and 2E. Specifically, when the membrane was perturbed, the molecule entered the cell, bound to DNA and fluoresced. The positive and negative controls were a quaternary ammonium compound (QAC; 12,3,2,3,12) and vancomycin, respectively. See the results shown in, e.g., FIGS. 1A-1F.

These data shows that compounds 1H and 3N perturb the membrane at 32 μM which is 4 dilutions higher than its MIC of 2 μM.

The disclosures of each patent, patent application, and publication cited or described in this document, including Solinski et al., “Honokiol-Inspired Analogs as Inhibitors of Oral Bacteria,” 2018, 4(2):118-122, are hereby incorporated herein by reference, each in its entirety, for all purposes.

1. A method of inhibiting, reducing, or ameliorating bacterial growth on a substrate, comprising contacting the substrate with a compound of formula (I): 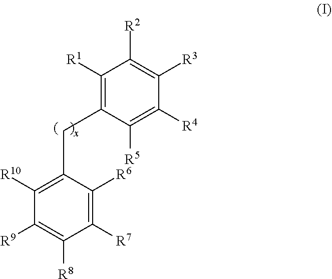
wherein:
R¹ and IV are, independently, H, OH, C₁₋₆alkyl, C₁₋₆alkoxy, C₁₋₆alkyl, or C₃₋₁₀cycloalkyl;
R² and R⁴ are, independently, H, OH, C₁₋₆alkyl, C₂₋₆alkenyl, C₁₋₆alkoxy, or C₃₋₁₀cycloalkyl;
R³ and R⁸ are, independently, H, OH, C₁₋₆alkyl, C₁₋₆alkoxy, C₃₋₁₀cycloalkyl, or optionally substituted aryl;
R⁶ and R¹⁰ are, independently, H, OH, C₁₋₆alkyl, C₁₋₆alkoxy, or C₃₋₁₀cycloalkyl;
R⁷ and R⁹ are, independently, H, OH, C₁₋₆alkyl, C₁₋₆alkoxy, C₂₋₆alkenyl, C₃₋₁₀cycloalkyl, or optionally substituted aryl;
or R⁹ and R¹⁰ are joined to form —C(R^{9′})═C(R¹¹)—C(R¹²)═C(R^{1′})—;
R^{9′}, R^{10′}, R¹¹ and R¹² are, independently, H, OH, halogen, C₁₋₆alkyl, C₁₋₆alkoxy, C₃₋₁₀cycloalkyl, C(O)(C₁₋₆alkyl), optionally substituted aryl, or optionally substituted heteroaryl; and
x is 0 to 8;
with the proviso that (i) at least one of R¹ to R⁵ is OH or at least one of R⁶ to R¹⁰ is OH; and (ii) the compound is not 3′,5-diallyl-[1,1′-biphenyl]-2,4′-diol,
or a pharmaceutically acceptable salt thereof.

2. The method of claim 1, wherein the compound is of formula (V): 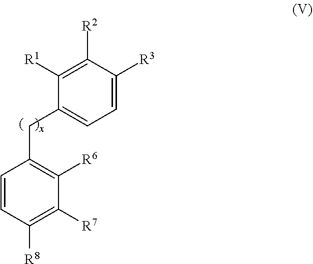
wherein:
R¹ is H, OH, or C₁₋₆alkyl;
R² is H, OH, C₁₋₆alkyl, or C₁₋₆alkoxy;
R³ is H, OH, C₁₋₈alkyl, or C₃₋₈cycloalkyl;
R⁶ is H, OH, or C₁₋₆alkyl;
R⁷ is H, OH, or C₁₋₆alkyl;
R⁸ is H, OH, C₁₋₈alkyl, or C₃₋₈cycloalkyl;
x is 0 to 8;
with the proviso that at least one of R¹ to R³ or R⁶ to R⁸ is OH;
or a pharmaceutically acceptable salt thereof.

3. The method of claim 2, wherein R¹ is H.

4. The method of claim 2, wherein one or more of R¹, R², R³, R⁶, R⁷, and R⁸ is C₁₋₆alkyl.

5. (canceled)

6. The method of claim 2, wherein R¹ is OH.

7. The method of claim 2, wherein R² is OH.

8. (canceled)

9. (canceled)

10. The method of claim 8, wherein one or both of R² and R⁸ is C₁₋₆alkoxy.

11-12. (canceled)

13. The method of claim 2, wherein R⁶ is OH.

14-15. (canceled)

16. The method of claim 2, wherein R⁷ is OH.

17-19. (canceled)

20. The method of claim 2, wherein R⁸ is H.

21. The method of claim 2, wherein R⁸ is OH.

22-24. (canceled)

25. The method of claim 2, wherein R¹, R³, and R⁶ are H, R¹ and R⁶ are H, or R³ and R⁸ are H.

26-27. (canceled)

28. The method of claim 2, wherein x is 2-6.

29. The method of claim 2, wherein one of R¹ to R³ or R⁶ to R⁸ is ^{t}Bu.

30. The method of claim 1, wherein the compound is of formula (IVA) or (IVB): 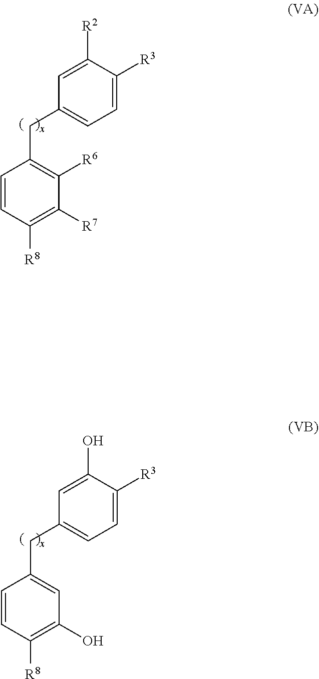
wherein:
R² is H, OH, C₁₋₆alkyl, or C₁₋₆alkoxy;
R³ is H, OH, C₁₋₆alkyl, or C₃₋₈cycloalkyl;
R⁷ is H, OH, or C₁₋₆alkyl;
R⁸ is H, OH, C₁₋₆alkyl, or C₃₋₈cycloalkyl;
x is 2-6.

31. (canceled)

32. The method of claim 1Q, wherein the compound is: 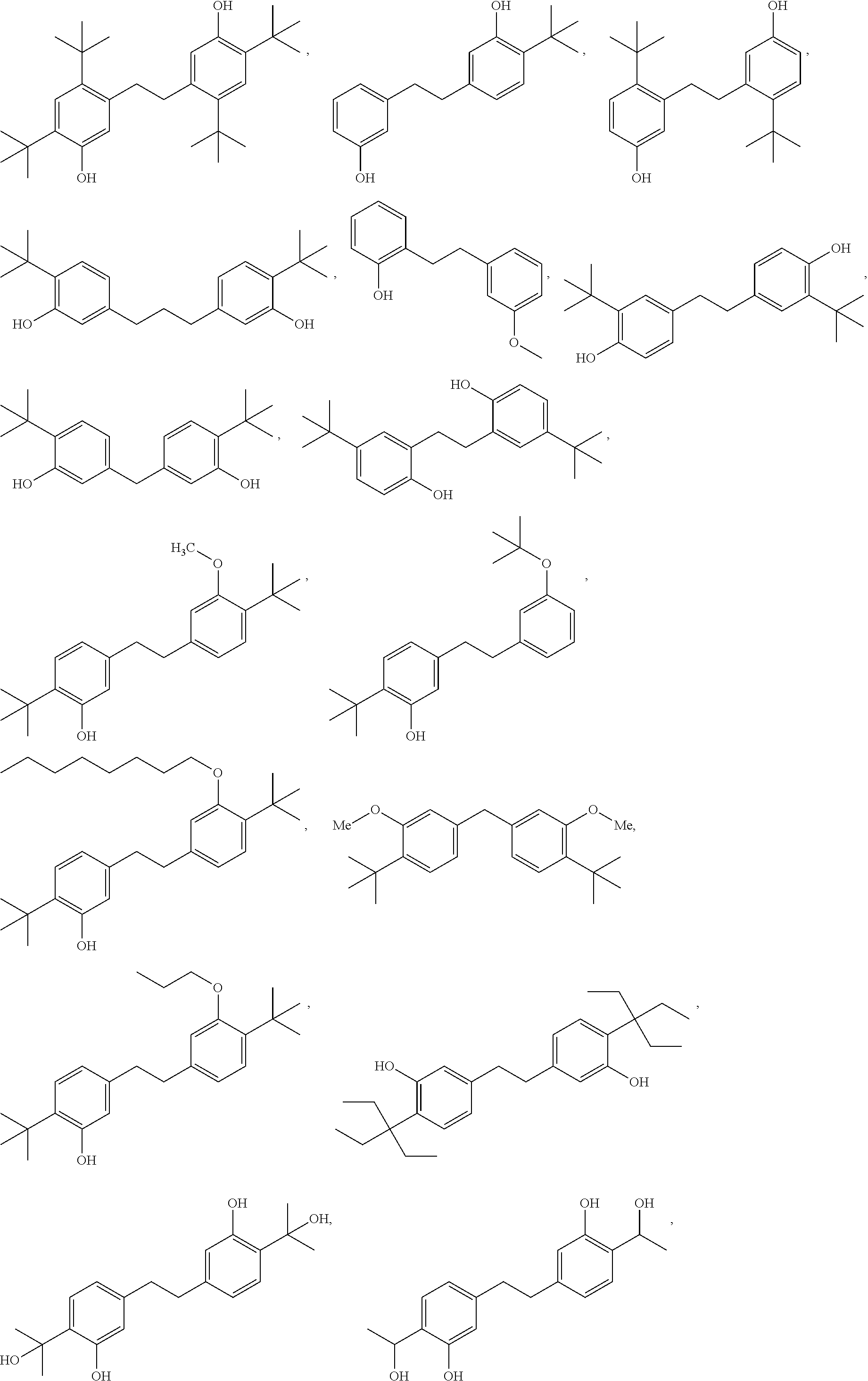 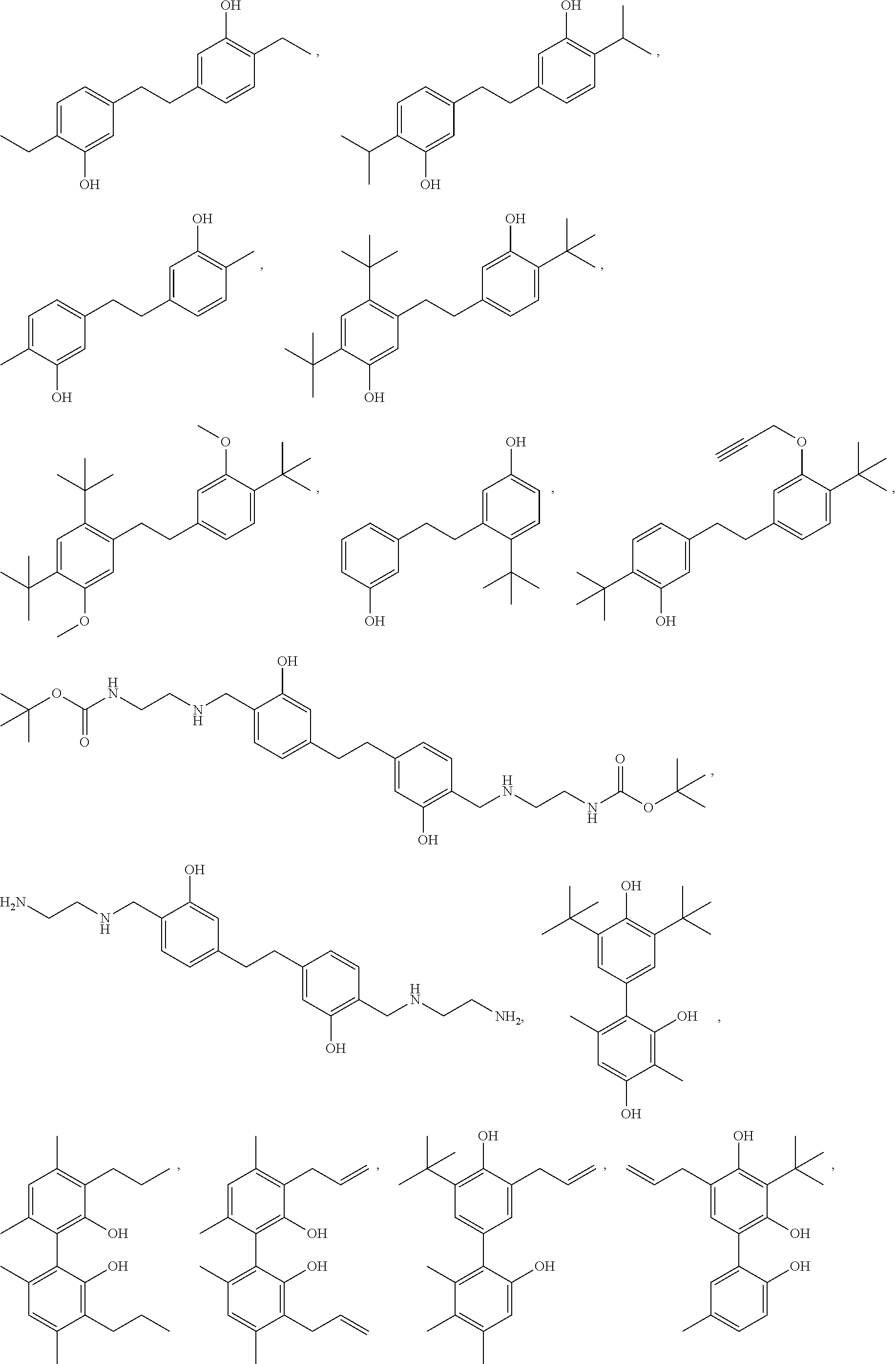 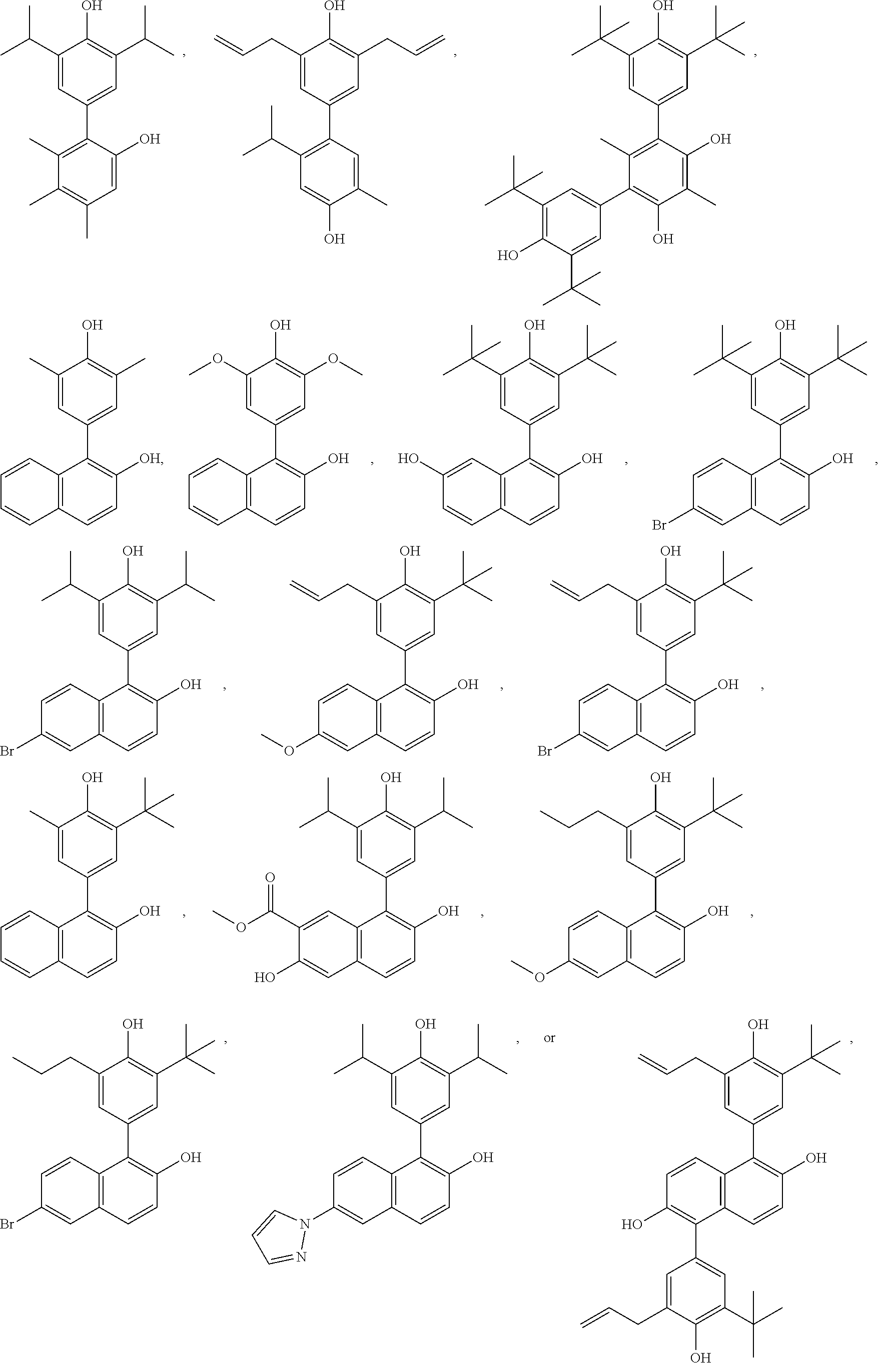 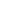
or a salt thereof.

33-34. (canceled)

35. The compound of claim 1, that is: 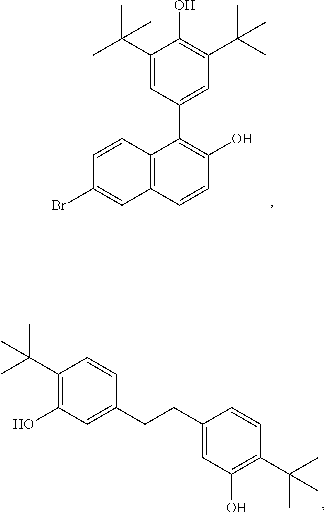
or a salt thereof.

36. (canceled)

37. The method of claim 1, wherein the bacterial growth is a biofilm.

38. The method of claim 1, wherein the substrate is skin or an oral cavity.

39. (canceled)

40. The method of claim 1, wherein the bacterium is an oral pathogenic bacterium.

41. The method of claim 1, wherein the bacterium is S. mutans, S. sanguinis, or S. gordonii.

42. (canceled)

43. A dental care product, soap, or antibacterial product, comprising one or more compounds of formula (V): 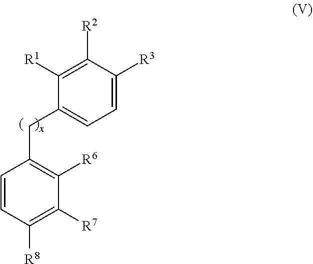
wherein:
R¹ is H, OH, or C₁₋₆alkyl;
R² is H, OH, C₁₋₆alkyl, or C₁₋₆alkoxy;
R³ is H, OH, C₁₋₈alkyl, or C₃₋₁₀cycloalkyl;
R⁶ is H, OH, or C₁₋₆alkyl;
R⁷ is H, OH, or C₁₋₆alkyl;
R⁸ is H, OH, C₁₋₈alkyl, or C₃₋₁₀cycloalkyl;
x is 0 to 8;
with the proviso that at least one of R¹ to R³ or R⁶ to R⁸ is OH;
or a pharmaceutically acceptable salt thereof.

44-45. (canceled)

46. A compound of formula (I): 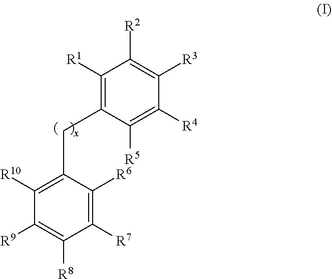
wherein:
R¹ and R⁵ are, independently, H, OH, C₁₋₈alkyl, or C₃₋₁₀cycloalkyl;
R² and R⁴ are, independently, H, OH, C₁₋₈alkyl, C₂₋₆alkenyl, C₁₋₆alkoxy, or C₃₋₁₀cycloalkyl;
R³ and R⁸ are, independently, H, OH, C₁₋₈alkyl, C₃₋₁₀cycloalkyl, and optionally substituted aryl;
R⁶ and R¹⁰ are, independently, H, OH, C₁₋₈alkyl, or C₃₋₁₀cycloalkyl;
R⁷ and R⁹ are, independently, H, OH, C₁₋₈alkyl, C₂₋₆alkenyl, C₃₋₈cycloalkyl, optionally substituted aryl;
or R⁹ and R¹⁰ are joined to form —C(R^{9′})═C(R¹¹)—C(R¹²)═C(R^{10′})—;
R^{9′}, R^{10′}, R¹¹ and R¹² are, independently, H, OH, halogen, C₁₋₆alkyl, C₁₋₆alkoxy, C₃₋₈cycloalkyl, C(O)(C₁₋₆alkyl), optionally substituted aryl, or optionally substituted heteroaryl; and
x is 0 to 8;
with the proviso that (i) at least one of R¹ to R⁵ is OH; (ii) at least one of R⁶ to R¹⁰ is OH; and (iii) the compound is not:
2-(3-methoxyphenethyl)phenol,
[1,1′-biphenyl]-2,2′-diol,
[1,1′-biphenyl]-2,2′-diol,
[1,1′-biphenyl]-2,4′-diol,
[1,1′-biphenyl]-3,3′-diol,
[1,1′-biphenyl]-4,4′-diol,
3,3′,6,6′-tetramethyl-[1,1′-biphenyl]-2,2′-diol,
2,2′,3,3′,6,6′-hexamethyl-[1,1′-biphenyl]-4,4′-diol;
3,3′,4,4′,6,6′-hexamethyl-[1,1′-biphenyl]-2,2′-diol,
3,3′-diallyl-4,4′,6,6′-tetramethyl-[1,1′-biphenyl]-2,2′-diol,
4,4′,6,6′-tetramethyl-3,3′-dipropyl-[1,1′-biphenyl]-2,2′-diol,
4,4′-dimethoxy-3,3′,6,6′-tetramethyl-[1,1′-biphenyl]-2,2′-diol,
[1,1′-biphenyl]-3,3′,4-triol,
[1,1′-biphenyl]-3,3′,4′,5′-tetraol,
3,3′,6,6′-tetramethyl-[1,1′-biphenyl]-2,2′,4,4′-tetraol,
4′,5″-dimethyl-[1,1′:2′,1″:2″,1″¹-quaterphenyl]-3′,4″,5′,6″-tetraol, or
3,3′-dimethyl-6,6′-di(oct-1-yn-1-yl)-[1,1′-biphenyl]-2,2′,4,4′-tetraol,
or a pharmaceutically acceptable salt thereof.

47-70. (canceled)

71. The compound of claim 46, that is: 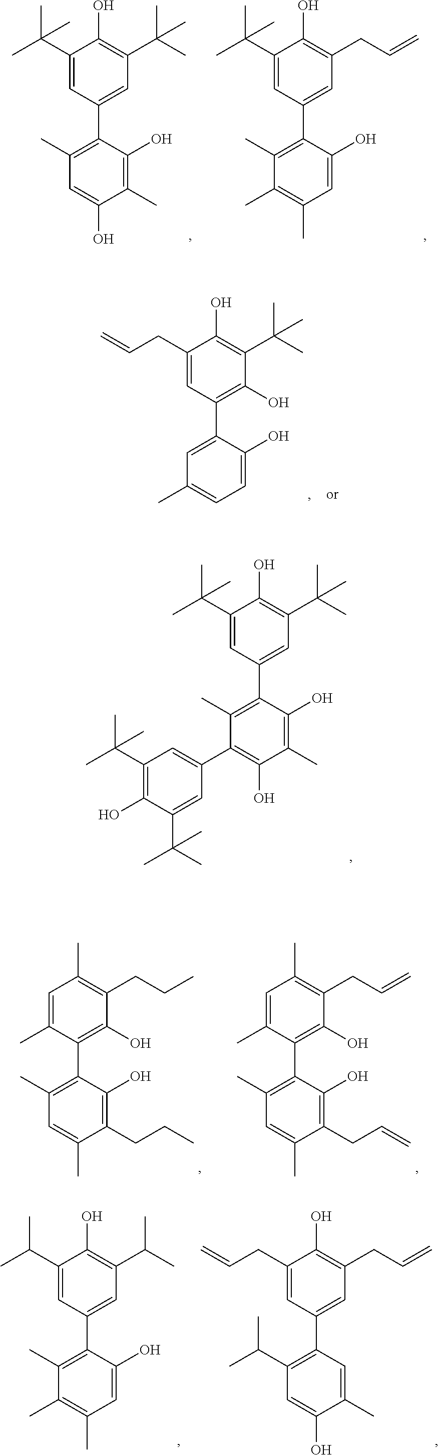 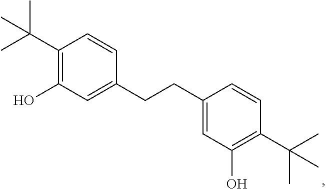 [SMILES: CC(C)(C)c1ccc(CCc2ccc(C(C)(C)C)c(O)c2)cc1O]
or a salt thereof.

72-73. (canceled)

74. A compound of formula (V): 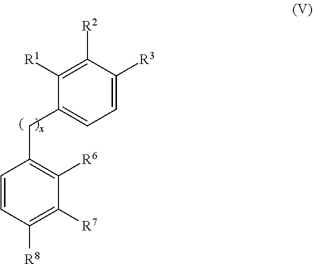
wherein:
R¹ is H, OH, or C₁₋₆alkyl;
R² is H, OH, C₁₋₆alkyl, or C₁₋₆alkoxy;
R³ is H, OH, C₁₋₈alkyl, or C₃₋₁₀cycloalkyl;
R⁶ is H, OH, or C₁₋₆alkyl;
R⁷ is H, OH, or C₁₋₆alkyl;
R⁸ is H, OH, C₁₋₈alkyl, or C₃₋₁₀cycloalkyl;
x is 0 to 8;
with the proviso that at least one of R¹ to R³ or R⁶ to R⁸ is OH;
or a pharmaceutically acceptable salt thereof.

75-103. (canceled)

104. The compound of claim 74, wherein the compound is:

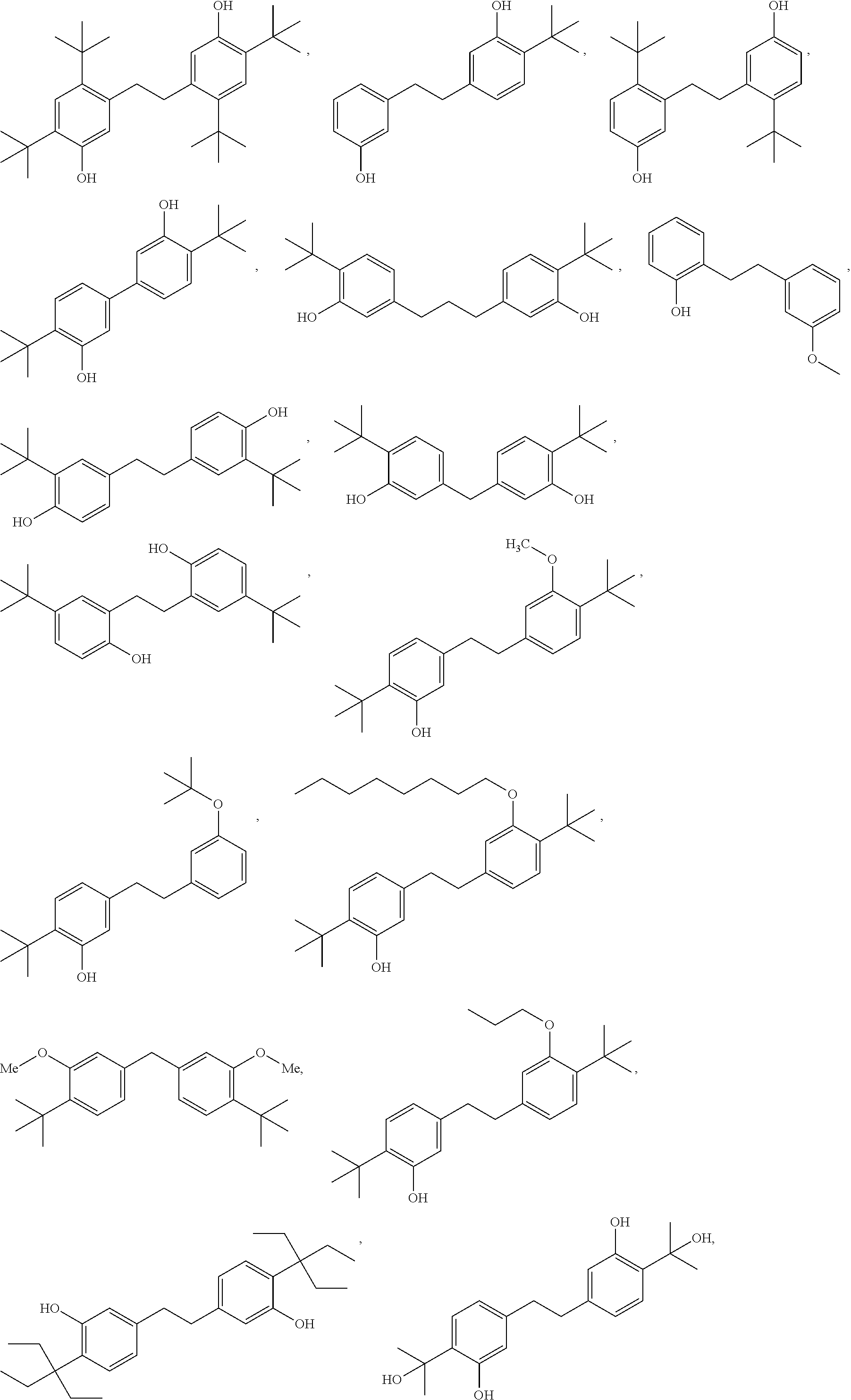

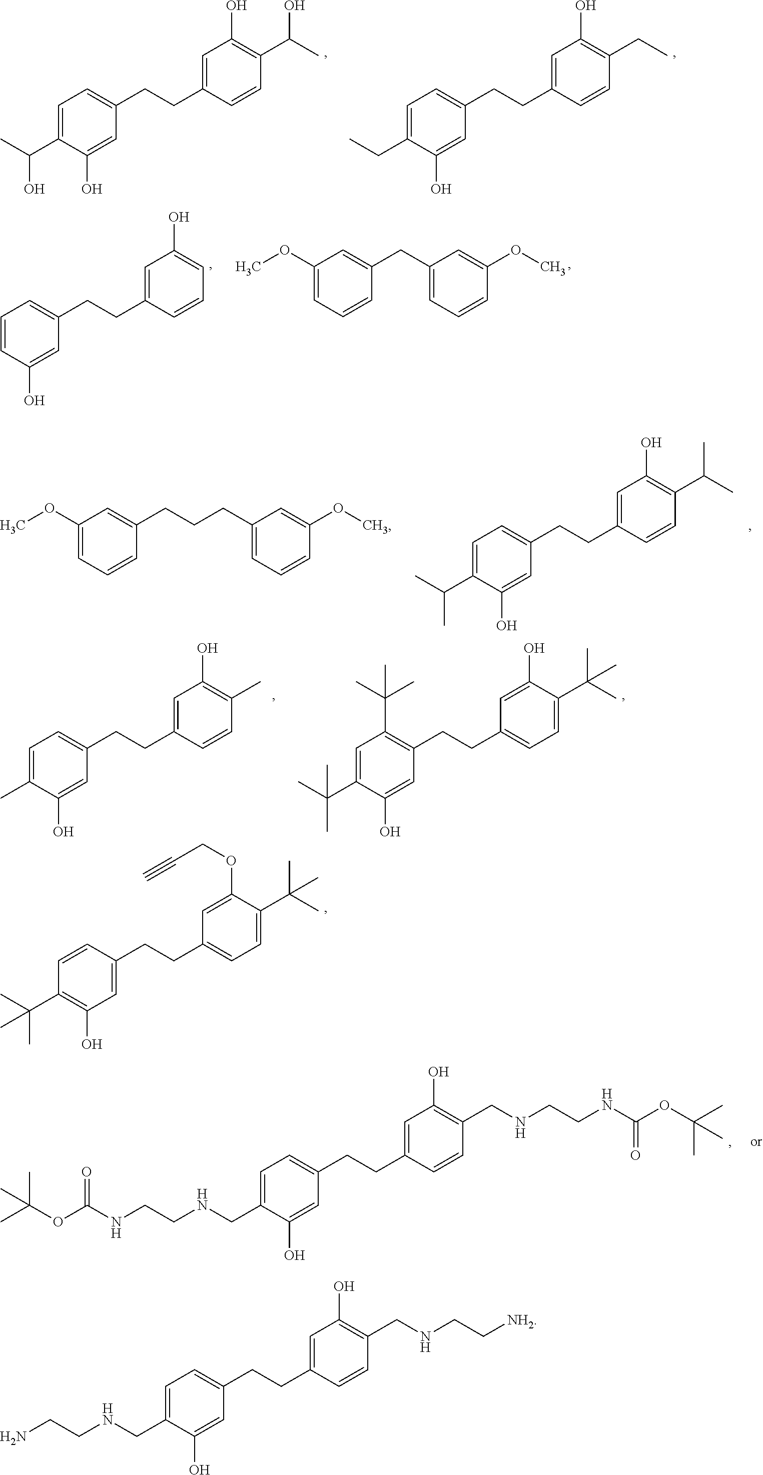

105-107. (canceled)

108. A composition comprising one or more compounds of claim 74 and an excipient.

109-113. (canceled)
